(19) United States
(12) Patent Application Publication
(10) Pub. No.: US 2008/0113344 A1
(43) Pub. Date: May 15, 2008

(54) Methods and Compositions for the Response Prediction of Malignant Neoplasia to Treatment

(76) Inventors: Ralph Wirtz, Koln (DE); Marc Munnes, Erkrath (DE)

Correspondence Address: CHOATE, HALL & STEWART LLP, TWO INTERNATIONAL PLACE, BOSTON, MA, 02110, US

(21) Appl. No.: 10/576,900

(22) PCT Filed: Oct. 15, 2004
(86) PCT No.: PCT/EP04/11599

§ 371 Date: Apr. 30, 2007

(30) Foreign Application Priority Data
Oct. 28, 2003 (EP) 03024565.8

(51) Int. Cl. C12Q 1/68

(52) U.S. Cl. 435/6

(57) ABSTRACT

The invention provides novel compositions, methods and uses, for the prediction, diagnosis, prognosis, prevention and treatment of malignant neoplasia and breast cancer in particular. Genes that are differentially expressed in breast tissue of breast cancer patients versus those of normal people are disclosed.

### TECHNICAL FIELD OF THE INVENTION

The invention relates to methods and compositions for the prediction, diagnosis, prognosis, prevention and treatment of neoplastic disease. Of particular interest is the response prediction of neoplastic lesions to various therapeutic regimens. Neoplastic disease is often caused by chromosomal rearrangements which lead to over- or underexpression of the rearranged genes. The invention discloses genes which are overexpressed in neoplastic tissue and are useful as diagnostic markers and targets for treatment. Methods are disclosed for predicting, diagnosing and prognosing as well as preventing and treating neoplastic disease.

### BACKGROUND OF THE INVENTION

Chromosomal aberrations (amplifications, deletions, inversions, insertions, translocations and/or viral integrations) are of importance for the development of cancer and neoplastic lesions, as they account for deregulations of the respective regions. Amplifications of genomic regions have been described, in which genes of importance for growth characteristics, differentiation, invasiveness or resistance to therapeutic intervention are located. One of those regions with chromosomal aberrations is the region carrying the HER-2/neu gene which is amplified in breast cancer patients. In approximately 25% of breast cancer patients the HER-2/neu gene is overexpressed due to gene amplification. HER-2/neu overexpression correlates with a poor prognosis (relapse, overall survival, sensitivity to therapeutics). The importance of HER-2/neu for the prognosis of the disease progression has been described [Gusterson et al., 1992, (1)]. Gene specific antibodies raised against HER-2/neu (Herceptin™) have been generated to treat the respective cancer patients. However, only about 50% of the patients benefit from the antibody treatment with Herceptin™, which is most often combined with chemotherapeutic regimen. The discrepancy of HER-2/neu positive tumors (overexpressing HER-2/neu to similar extent) with regard to responsiveness to therapeutic intervention suggest, that there might be additional factors or genes being involved in growth and apoptotic characteristics of the respective tumor tissues. There seems to be no monocausal relationship between overexpression of the growth factor receptor HER-2/neu and therapy outcome. In line with this the measurement of commonly used tumor markers such as estrogen receptor, progesterone receptor, p53 and Ki-67 do provide only very limited information on clinical outcome of specific therapeutic decisions. Therefore there is a great need for a more detailed diagnostic and prognostic classification of tumors to enable improved therapy decisions and prediction of survival of the patients. The present invention addresses the need for additional markers by providing genes, which expression is deregulated in tumors and correlates with clinical outcome. One focus is the deregulation of genes present in specific chromosomal regions and their interaction in disease development and drug responsiveness.

HER-2/neu and other markers for neoplastic disease are commonly assayed with diagnostic methods such as immunohistochemistry (IHC) (e.g. HercepTest™ from DAKO Inc.) and Fluorescence-In-Situ-Hybridization (FISH) (e.g. quantitative measurement of the HER-2/neu and Topoisomerase II alpha with a fluorescence-in-situ-Hybridization kit from VYSIS). Additionally HER-2/neu can be assayed by detecting HER-2/neu fragments in serum with an ELISA test (BAYER Corp.) or a with a quantitative PCR kit which compares the amount of HER-2/neu gene with the amount of a non-amplified control gene in order to detect HER-2/neu gene amplifications (ROCHE). These methods, however, exhibit multiple disadvantages with regard to sensitivity, specificity, technical and personnel efforts, costs, time consumption, inter-lab reproducibility. These methods are also restricted with regard to measurement of multiple parameters within one patient sample (“multiplexing”). Usually only about 3 to 4 parameters (e.g. genes or gene products) can be detected per tissue slide. Therefore, there is a need to develop a fast and simple test to measure simultaneously multiple parameters in one sample. The present invention addresses the need for a fast and simple high-resolution method, that is able to detect multiple diagnostic and prognostic markers simultaneously.

### SUMMARY OF THE INVENTION

The present invention is based on discovery that chromosomal alterations in cancer tissues can lead to changes in the expression of genes that are encoded by the altered chromosomal regions. Exemplary 43 human genes have been identified that are co-amplified in neoplastic lesions from breast cancer tissue resulting in altered expression of several of these genes (Tables 1 to 4). These 43 genes are differentially expressed in breast cancer states, relative to their expression in normal, or non-breast cancer states. The present invention relates to derivatives, fragments, analogues and homologues of these genes and uses or methods of using of the same.

The present invention further relates to novel preventive, predictive, diagnostic, prognostic and therapeutic compositions and uses for malignant neoplasia and breast cancer in particular. Especially membrane bound marker gene products containing extracellular domains can be a particularly useful target for treatment methods as well as diagnostic and clinical monitoring methods.

It is a discovery of the present invention that several of these genes are characterized in that their gene products functionally interact in signaling cascades or by directly or indirectly influencing each other. This interaction is important for the normal physiology of certain non-neoplastic tissues (e.g. brain or neurogenic tissue). The deregulation of these genes in neoplastic lesions where they are normally exhibit of different level of activity or are not active, however, results in pathophysiology and affects the characteristics of the disease-associated tissue.

The present invention further relates to methods for detecting these deregulations in malignant neoplasia on DNA and mRNA level.

The present invention further relates to a method for the detection of chromosomal alterations characterized in that the relative abundance of individual mRNAs, encoded by genes, located in altered chromosomal regions is detected.

The present invention further relates to a method for the detection of the flanking breakpoints of named chromosomal alterations by measurement of DNA copy number by quantitative PCR or DNA-Arrays and DNA sequencing.

A method for the prediction, diagnosis or prognosis of malignant neoplasia by the detection of DNA sequences flanking named genomic breakpoint or are located within such.

The present invention further relates to a method for the detection of chromosomal alterations characterized in that the copy number of one or more genomic nucleic acid sequences located within an altered chromosomal region(s) is detected by quantitative PCR techniques (e.g. TaqMan™, Lightcycler™ and iCycler™).

The present invention further relates to a method for the prediction, diagnosis or prognosis of malignant neoplasia by the detection of at least 2 markers whereby the markers are genes and fragments thereof or genomic nucleic acid sequences that are located on one chromosomal region which is altered in malignant neoplasia and breast cancer in particular.

The present invention also discloses a method for the prediction, diagnosis or prognosis of malignant neoplasia by the detection of at least 2 markers whereby the markers are located on one or more chromosomal region(s) which is/are altered in malignant neoplasia; and the markers interact as (i) receptor and ligand or (ii) members of the same signal transduction pathway or (iii) members of synergistic signal transduction pathways or (iv) members of antagonistic signal transduction pathways or (v) transcription factor and transcription factor binding site.

Also disclosed is a method for the prediction, diagnosis or prognosis of malignant neoplasia by the detection of at least one marker whereby the marker is a VNTR, SNP, RFLP or STS which is located on one chromosomal region which is altered in malignant neoplasia due to amplification and the marker is detected in (a) a cancerous and (b) a non cancerous tissue or biological sample from the same individual. A preferred embodiment is the detection of at least one VNIR marker of Table 6 or at least on SNP marker of Table 4 or combinations thereof. Even more preferred can the detection, quantification and sizing of such polymorphic markers be achieved by methods of (a) for the comparative measurement of amount and size by PCR amplification and subsequent capillary electrophoresis, (b) for sequence determination and allelic discrimination by gel electrophoresis (e.g. SSCP, DGGE), real time kinetic PCR, direct DNA sequencing, pyro-sequencing, mass-specific allelic discrimination or resequencing by DNA array technologies, (c) for the determination of specific restriction patterns and subsequent electrophoretic separation and (d) for allelic discrimination by allel specific PCR (e.g. ASO). An even more favorable detection of a hetrozygous VNTR, SNP, RFLP or STS is done in a multiplex fashion, utilizing a variety of labeled primers (e.g. fluorescent, radioactive, bioactive) and a suitable capillary electrophoresis (CE) detection system.

In another embodiment the expression of these genes can be detected with DNA-arrays as described in WO9727317 and U.S. Pat. No. 6,379,895.

In a further embodiment the expression of these genes can be detected with bead based direct fluorescent readout techniques such as described in WO9714028 and WO9952708.

In one embodiment, the invention pertains to a method of determining the phenotype of a cell or tissue, comprising detecting the differential expression, relative to a normal or untreated cell, of at least one polynucleotide comprising SEQ ID NO: 2 to 6, 8, 9, 11 to 16, 18, 19 or 21 to 26 or 53 to 75, wherein the polynucleotide is differentially expressed by at least about 1.5 fold, at least about 2 fold or at least about 3 fold.

In a further aspect the invention pertains to a method of determining the phenotype of a cell or tissue, comprising detecting the differential expression, relative to a normal or untreated cell, of at least one polynucleotide which hybridizes under stringent conditions to one of the polynucleotides of SEQ ID NO: 2 to 6, 8, 9, 11 to 16, 18, 19 or 21 to 26 or 53 to 75 and encodes a polypeptide exhibiting the same biological function as given in Table 2 or 3 for the respective polynucleotide, wherein the polynucleotide is differentially expressed by at least at least about 1.5 fold, at least about 2 fold or at least about 3 fold.

In another embodiment of the invention a polynucleotide comprising a polynucleotide selected from SEQ ID NO: 2 to 6, 8, 9, 11 to 16, 18, 19 or 21 to 26 and 53 to 75 or encoding one of the polypeptides with SEQ ID NO: 28 to 32, 34, 35, 37 to 42, 44, 45 or 47 to 52 or 76 to 98 can be used to identify cells or tissue in individuals which exhibit a phenotype predisposed to breast cancer or a diseased phenotype, thereby (a) predicting whether an individual is at risk for the development, or (b) diagnosing whether an individual is having, or (c) prognosing the progression or the outcome of the treatment malignant neoplasia and breast cancer in particular.

In yet another embodiment the invention provides a method for identifying genomic regions which are altered on the chromosomal level and encode genes that are linked by function and are differentially expressed in malignant neoplasia and breast cancer in particular.

In yet another embodiment the invention provides the genomic regions 17q21, 3p21 and 12q13 for use in prediction, diagnosis and prognosis as well as prevention and treatment of malignant neoplasia and breast cancer. In particular not only the intragenic regions, but also intergenic regions, pseudogenes or non-transcribed genes of said chromosomal regions can be used for diagnostic, predictive, prognostic and preventive and therapeutic compositions and methods. Therefore sequences of coding or non-coding regions as depicted in this invention are offered by way of illustration and not by way of limitation. As one aspect of this, genomic sequences in between the genomic sequences depicted can be used for similar purposes.

In yet another embodiment the invention provides methods of screening for agents which regulate the activity of a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75. A test compound is contacted with a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75. Binding of the test compound to the polypeptide is detected. A test compound which binds to the polypeptide is thereby identified as a potential therapeutic agent for the treatment of malignant neoplasia and more particularly breast cancer.

In even another embodiment the invention provides another method of screening for agents which regulate the activity of a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75. A test compound is contacted with a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75. A biological activity mediated by the polypeptide is detected. A test compound which decreases the biological activity is thereby identified as a potential therapeutic agent for decreasing the activity of the polypeptide encoded by a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 in malignant neoplasia and breast cancer in particular. A test compound which increases the biological activity is thereby identified as a potential therapeutic agent for increasing the activity of the polypeptide encoded by a polypeptide selected from one of the polypeptides with SEQ ID NO: 27 to 52 and 76 to 98 or encoded by a polynucleotide comprising a polynucleotide selected from SEQ ID NO: 1 to 26 and 53 to 75 in malignant neoplasia and breast cancer in particular.

In another embodiment the invention provides a method of screening for agents which regulate the activity of a polynucleotide comprising a polynucleotide selected from SEQ ID NO: 1 to 26 and 53 to 75. A test compound is contacted with a polynucleotide comprising a polynucleotide selected from SEQ ID NO: 1 to 26 and 53 to 75. Binding of the test compound to the polynucleotide comprising a polynucleotide selected from SEQ ID NO: 1 to 26 and 53 to 75 is detected. A test compound which binds to the polynucleotide is thereby identified as a potential therapeutic agent for regulating the activity of a polynucleotide comprising a polynucleotide selected from SEQ ID NO: 1 to 26 and 53 to 75 in malignant neoplasia and breast cancer in particular.

The invention thus provides polypeptides selected from one of the polypeptides with SEQ ID NO: 27 to 52 and 76 to 98 or encoded by a polynucleotide comprising a polynucleotide selected from SEQ ID NO: 1 to 26 and 53 to 75 which can be used to identify compounds which may act, for example, as regulators or modulators such as agonists and antagonists, partial agonists, inverse agonists, activators, co-activators and inhibitors of the polypeptide comprising a polypeptide selected from SEQ ID NO: 27 to 52 and 76 to 98 or encoded by a polynucleotide comprising a polynucleotide selected from SEQ ID NO: 1 to 26 and 53 to 75. Accordingly, the invention provides reagents and methods for regulating a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 in malignant neoplasia and more particularly breast cancer. The regulation can be an up- or down regulation. Reagents that modulate the expression, stability or amount of a polynucleotide comprising a polynucleotide selected from SEQ ID NO: 1 to 26 and 53 to 75 or the activity of the polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 can be a protein, a peptide, a peptidomimetic, a nucleic acid, a nucleic acid analogue (e.g. peptide nucleic acid, locked nucleic acid) or a small molecule. Methods that modulate the expression, stability or amount of a polynucleotide comprising a polynucleotide selected from SEQ ID NO: 1 to 26 and 53 to 75 or the activity of the polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 can be gene replacement therapies, antisense, ribozyme and triplex nucleic acid approaches.

In one embodiment of the invention provides antibodies which specifically bind to a full-length or partial polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 or a polynucleotide comprising a polynucleotide selected from SEQ ID NO: 1 to 26 and 53 to 75 for use in prediction, prevention, diagnosis, prognosis and treatment of malignant neoplasia and breast cancer in particular.

Yet another embodiment of the invention is the use of a reagent which specifically binds to a polynucleotide comprising a polynucleotide selected from SEQ ID NO: 1 to 26 and 53 to 75 or a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 in the preparation of a medicament for the treatment of malignant neoplasia and breast cancer in particular.

Still another embodiment is the use of a reagent that modulates the activity or stability of a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75 or the expression, amount or stability of a polynucleotide comprising a polynucleotide selected from SEQ ID NO: 1 to 26 and 53 to 75 in the preparation of a medicament for the treatment of malignant neoplasia and breast cancer in particular.

Still another embodiment of the invention is a pharmaceutical composition which includes a reagent which specifically binds to a polynucleotide comprising a polynucleotide selected from SEQ ID NO: 1 to 26 and 53 to 75 or a polypeptide comprising a polypeptide selected from SEQ ID NO: 27 to 52 and 76 to 98 or encoded by a polynucleotide comprising a polynucleotide selected from SEQ ID NO: 1 to 26 and 53 to 75, and a pharmaceutically acceptable carrier.

Yet another embodiment of the invention is a pharmaceutical composition including a polynucleotide comprising a polynucleotide selected from SEQ ID NO: 1 to 26 and 53 to 75 or encoding a polypeptide comprising a polypeptide selected from SEQ ID NO: 27 to 52 and 76 to 98.

In one embodiment, a reagent which alters the level of expression in a cell of a polynucleotide comprising a polynucleotide selected from SEQ ID NO: 1 to 26 and 53 to 75 or encoding a polypeptide comprising a polypeptide selected from SEQ ID NO: 27 to 52 and 76 to 98, or a sequence complementary thereto, is identified by providing a cell, treating the cell with a test reagent, determining the level of expression in the cell of a polynucleotide comprising a polynucleotide selected from SEQ ID NO: 1 to 26 and 53 to 75 or encoding a polypeptide comprising a polypeptide selected from SEQ ID NO: 27 to 52 and 76 to 98 or a sequence complementary thereto, and comparing the level of expression of the polynucleotide in the treated cell with the level of expression of the polynucleotide in an untreated cell, wherein a change in the level of expression of the polynucleotide in the treated cell relative to the level of expression of the polynucleotide in the untreated cell is indicative of an agent which alters the level of expression of the polynucleotide in a cell.

The invention further provides a pharmaceutical composition comprising a reagent identified by this method.

Another embodiment of the invention is a pharmaceutical composition which includes a polypeptide comprising a polypeptide selected from SEQ ID NO: 27 to 52 and 76 to 98 or which is encoded by a polynucleotide comprising a polynucleotide selected from SEQ ID NO: 1 to 26 and 53 to 75.

A further embodiment of the invention is a pharmaceutical composition comprising a polynucleotide including a sequence which hybridizes under stringent conditions to a polynucleotide comprising a polynucleotide selected from SEQ ID NO: 1 to 26 and 53 to 75 and encoding a polypeptide exhibiting the same biological function as given for the respective polynucleotide in Table 2 or 3, or encoding a polypeptide comprising a polypeptide selected from SEQ ID NO: 27 to 52 and 76 to 98. Pharmaceutical compositions, useful in the present invention may further include fusion proteins comprising a polypeptide comprising a polynucleotide selected from SEQ ID NO: 27 to 52 and 76 to 98, or a fragment thereof, antibodies, or antibody fragments

### BRIEF DESCRIPTION OF THE DRAWINGS

FIG. 1 shows a sketch of the chromosome 17 with G-banding pattern and cytogenetic positions. In the blow out at the lower part of the figure a detailed view of the chromosomal area of the long arm of chromosome 17 (17q12-21.1) is provided. Each vertical rectangle depicted in medium gray, represents a gene as labeled below or above the individual position. The order of genes depicted in this graph has been deduced from experiments questioning the amplification an over expression and from public available data (e.g. UCSC, NCBI or Ensemble).

FIG. 2 shows the same region as depicted before in FIG. 1 and a cluster representation of the individual expression values measured by DNA-chip hybridization. The gene representing squares are indicated by a dotted line. In the upper part of the cluster representation 4 tumor cell lines, of which two harbor a known HER-2/neu over expression (SKBR3 and AU565), are depicted with their individual expression profiles. Not only the HER-2/neu gene shows a clear over expression but as provided by this invention several other genes with in the surrounding. In the middle part of the cluster representation expression data obtained from immune histochemically characterized tumor samples are presented. Two of the depicted probes show a significant over expression of genes marked by the white rectangles. For additional information and comparison expression profiles of several non diseased human tissues (mas obtained from Clontech Inc.) Are provided. Closest relation to the expression profile of HER-2/neu positive tumors displays human brain and neural tissue.

FIG. 3 provides data from DNA amplification measurements by qpcr (e.g. Taqman). Data indicates that in several analyzed breast cancer cell lines harbor amplification of genes which were located in the previously described region (ARCHEON). Data were displayed for each gene on the x-axis and 40-Ct at the y-axis. Data were normalized to the expression level of GAPDH as seen in the first group of columns.

FIG. 4 represents a graphical overview on the amplified regions and provides information on the length of the individual amplification and over expression in the analyzed tumor cell lines. The length of the amplification and the composition of genes has a significant impact on the nature of the cancer cell and on the responsiveness on certain drugs, as described elsewhere.

### DETAILED DESCRIPTION OF THE INVENTION

### Definitions

“Differential expression”, as used herein, refers to both quantitative as well as qualitative differences in the genes' expression patterns depending on differential development and/or tumor growth. Differentially expressed genes may represent “marker genes,” and/or “target genes”. The expression pattern of a differentially expressed gene disclosed herein may be utilized as part of a prognostic or diagnostic breast cancer evaluation. Alternatively, a differentially expressed gene disclosed herein may be used in methods for identifying reagents and compounds and uses of these reagents and compounds for the treatment of breast cancer as well as methods of treatment.

“Biological activity” or “bioactivity” or “activity” or “biological function”, which are used interchangeably, herein mean an effector or antigenic function that is directly or indirectly performed by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers a biological molecule, e.g., a nucleic acid, peptide, hormone, etc., whose presence or concentration can be detected and correlated with a known condition, such as a disease state.

“Marker gene,” as used herein, refers to a differentially expressed gene which expression pattern may be utilized as part of predictive, prognostic or diagnostic malignant neoplasia or breast cancer evaluation, or which, alternatively, may be used in methods for identifying compounds useful for the treatment or prevention of malignant neoplasia and breast cancer in particular. A marker gene may also have the characteristics of a target gene.

“Target gene”, as used herein, refers to a differentially expressed gene involved in breast cancer in a manner by which modulation of the level of target gene expression or of target gene product activity may act to ameliorate symptoms of malignant neoplasia and breast cancer in particular. A target gene may also have the characteristics of a marker gene.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, cell-containing bodyfluids, free floating nucleic acids, urine, peritoneal fluid, and pleural fluid, or cells therefrom. Biological samples may also include sections of tissues such as frozen sections taken for histological purposes.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide, a ligand or an enzyme substrate. An antagonist can also be a compound that downregulates expression of a gene or which reduces the amount of expressed protein present.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation (i.e., activation or stimulation (e.g., by agonizing or potentiating) and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “nucleotide analog” refers to oligomers or polymers being at least in one feature different from naturally occurring nucleotides, oligonucleotides or polynucleotides, but exhibiting functional features of the respective naturally occurring nucleotides (e.g. base paring, hybridization, coding information) and that can be used for said compositions. The nucleotide analogs can consist of non-naturally occurring bases or polymer backbones, examples of which are LNAs, PNAs and Morpholinos. The nucleotide analog has at least one molecule different from its naturally occurring counterpart or equivalent.

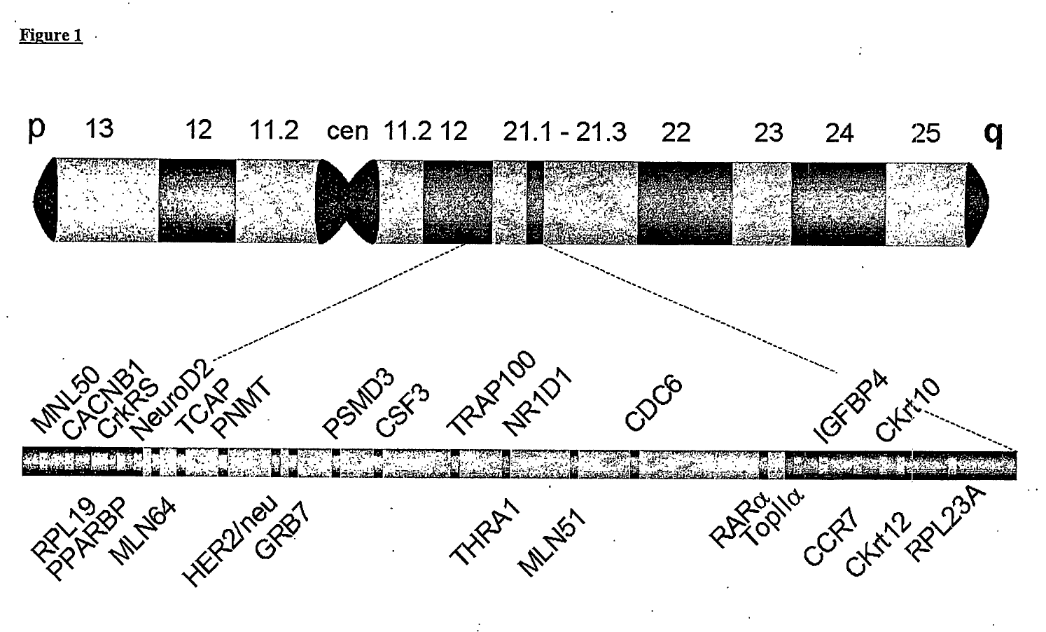

“BREAST CANCER GENES” or “BREAST CANCER GENE” as used herein refers to the polynucleotides of SEQ ID NO: 1 to 26 and 53 to 75, as well as derivatives, fragments, analogs and homologues thereof, the polypeptides encoded thereby, the polypeptides of SEQ ID NO: 27 to 52 and 76 to 98 as well as derivatives, fragments, analogs and homologues thereof and the corresponding genomic transcription units which can be derived or identified with standard techniques well known in the art using the information disclosed in Tables 1 to 5 and FIGS. 1 to 4. The GenBank, Locuslink ID and the UniGene accession numbers of the polynucleotide sequences of the SEQ ID NO: 1 to 26 and 53 to 75 and the polypeptides of the SEQ ID NO: 27 to 52 and 76 to 98 are shown in Table 1, the gene description, gene function and subcellular localization is given in Tables 2 and 3.

The term “chromosomal region” as used herein refers to a consecutive DNA stretch on a chromosome which can be defined by cytogenetic or other genetic markers such as e.g. restriction length polymorphisms (RFLPs), single nucleotide polymorphisms (SNPs), expressed sequence tags (ESTs), sequence tagged sites (STSs), microsatellites, variable number of tandem repeats (VNTRs) and genes. Typically a chromosomal region consists of up to 2 Megabases (MB), up to 4 MB, up to 6 MB, up to 8 MB, up to 10 MB, up to 20 MB or even more MB.

The term “altered chromosomal region” or “abberant chromosomal region” refers to a structural change of the chromosomal composition and DNA sequence, which can occur by the following events: amplifications, deletions, inversions, insertions, translocations and/or viral integrations. A trisomy, where a given cell harbors more than two copies of a chromosome, is within the meaning of the term “amplification” of a chromosome or chromosomal region.

The present invention provides polynucleotide sequences and proteins encoded thereby, as well as probes derived from the polynucleotide sequences, antibodies directed to the encoded proteins, and predictive, preventive, diagnostic, prognostic and therapeutic uses for individuals which are at risk for or which have malignant neoplasia and breast cancer in particular. The sequences disclosure herein have been found to be differentially expressed in samples from breast cancer.

The present invention is based on the identification of 43 genes that are differentially regulated (up- or downregulated) in tumor biopsies of patients with clinical evidence of breast cancer. The identification of 43 human genes which were not known to be differentially regulated in breast cancer states and their significance for the disease is described in the working examples herein. The characterization of the co-expression of these genes provides newly identified roles in breast cancer. The gene names, the database accession numbers (GenBank and UniGene) as well as the putative or known functions of the encoded proteins and their subcellular localization are given in Tables 1 to 4. The primer sequences used for the gene amplification are shown in Table 5.

In either situation, detecting expression of these genes in excess or in with lower level as compared to normal expression provides the basis for the diagnosis of malignant neoplasia and breast cancer. Furthermore, in testing the efficacy of compounds during clinical trials, a decrease in the level of the expression of these genes corresponds to a return from a disease condition to a normal state, and thereby indicates a positive effect of the compound.

Another aspect of the present invention is based on the observation that neighboring genes within defined genomic regions functionally interact and influence each others function directly or indirectly. A genomic region encoding functionally interacting genes that are co-amplified and co-expressed in neoplastic lesions has been defined as an “ARCHEON”. (ARCHEON=Altered Region of Changed Chromosomal Expression Observed in Neoplasms). Chromosomal alterations often affect more than one gene. This is true for amplifications, duplications, insertions, integrations, inversions, translocations, and deletions. These changes can have influence on the expression level of single or multiple genes. Most commonly in the field of cancer diagnostics and treatment the changes of expression levels have been investigated for single, putative relevant target genes such as MLVI2 (5p14), NRASL3 (6p12), EGFR (7p12), c-myc (8q23), Cyclin D1 (11q13), IGF1R (15q25), HER-2/neu (17q21), PCNA (20q12). However, the altered expression level and interaction of multiple (i.e. more than two) genes within one genomic region with each other has not been addressed. Genes of an ARCHEON form gene clusters with tissue specific expression patterns. The mode of interaction of individual genes within such a gene cluster suspected to represent an ARCHEON can be either protein-protein or protein-nucleic acid interaction, which may be illustrated but not limited by the following examples: ARCHEON gene interaction may be in the same signal transduction pathway, may be receptor to ligand binding, receptor kinase and SH2 or SH3 binding, transcription factor to promoter binding, nuclear hormone receptor to transcription factor binding, phospho group donation (e.g. kinases) and acceptance (e.g. phosphoprotein), mRNA stabilizing protein binding and transcriptional processes. The individual activity and specificity of a pair genes and or the proteins encoded thereby or of a group of such in a higher order, may be readily deduced from literature, published or deposited within public databases by the skilled person. However in the context of an ARCHEON the interaction of members being part of an ARCHEON will potentiate, exaggerate or reduce their singular functions. This interaction is of importance in defined normal tissues in which they are normally co-expressed. Therefore, these clusters have been commonly conserved during evolution. The aberrant expression of members of these ARCHEON in neoplastic lesions, however, (especially within tissues in which they are normally not expressed) has influence on tumor characteristics such as growth, invasiveness and drug responsiveness. Due to the interaction of these neighboring genes it is of importance to determine the members of the ARCHEON which are involved in the deregulation events. In this regard amplification and deletion events in neoplastic lesions are of special interest.

The invention relates to a method for the detection of chromosomal alterations by (a) determining the relative mRNA abundance of individual mRNA species or (b) determining the copy number of one or more chromosomal region(s) by quantitative PCR. In one embodiment information on the genomic organization and spatial regulation of chromosomal regions is assessed by bioinformatic analysis of the sequence information of the human genome (UCSC, NCBI) and then combined with RNA expression data from GeneChip™ DNA-Arrays (Affymetrix) and/or quantitative PCR (TaqMan) from RNA-samples or genomic DNA.

In a further embodiment the functional relationship of genes located on a chromosomal region which is altered (amplified or deleted) is established. The altered chromosomal region is defined as an ARCHEON if genes located on that region functionally interact.

The 17q21 locus was investigated as one model system, harboring the HER-2/neu gene. By establishing a high-resolution assay to detect amplification events in neighboring genes, 43 genes that are commonly co-amplified in breast cancer cell lines and patient samples were identified. By gene array technologies and immunological methods their co-overexpression in tumor samples was demonstrated. Surprisingly, by clustering tissue samples with HER-2/neu positive Tumor samples, it was found that the expression pattern of this larger genomic region (consisting of 43 genes) is very similar to control brain tissue. HER-2/neu negative breast tumor tissue did not show a similar expression pattern. Indeed, some of the genes within these cluster are important for neural development (HER-2/neu, THRA) in mouse model systems or are described to be expressed in neural cells (NeuroD2). Moreover, by searching similar gene combinations in the human and rodent genome additional homologous chromosomal regions on chromosome 3p21 and 12q13 harboring several isoforms of the respective genes (see below) were found. There was a strong evidence for multiple interactions between the 43 candidate genes, as being part of identical pathways (HER-2, neu, GRB7, CrkRS, CDC6), influencing the expression of each other (HER-2/neu, THRA, RARA), interacting with each other (PPARGBP, THRA, RARA, NR1D1 or HER-2/neu, GRB7) or expressed in defined tissues (CACNB1, PPARGBP, etc.). Interestingly, the genomic regions of the ARCHEONs that were identified are amplified in acquired Tamoxifen resistance of HER-2/neu negative cells (MCF7), which are normally sensitive to Tamoxifen treatment [Achuthan et al., 2001, (2)].

Moreover, altered responsiveness to treatment due to the alterations of the genes within these ARCHEONs was observed. Surprisingly, genes within the ARCHEONs are of importance even in the absence of HER-2/neu homologues. Some of the genes within the ARCHEONs, do not only serve as marker genes for prognostic purposes, but have already been known as targets for therapeutic intervention. For example TOP2 alpha is a target of anthracyclins. THRA and RARA can be targeted by hormones and hormone analogs (e.g. T3, rT3, RA). Due to their high affinity binding sites and available screening assays (reporter assays based on their transcriptional potential) the hormone receptors which are shown to be linked to neoplastic pathophysiology for the first time herein are ideal targets for drug screening and treatment of malignant neoplasia and breast cancer in particular. In this regard it is essential to know which members of the ARCHEON are altered in the neoplastic lesions. Particularly it is important to know the nature, number and extent to which the ARCHEON genes are amplified or deleted. The ARCHEONs are flanked by similar, endogenous retroviruses (e.g. HERV-K=“human endogenous retrovirus”), some of which are activated in breast cancer. These viruses may have also been involved in the evolutionary duplication of the ARCHEONs.

The analysis of the 17q21 region proved data obtained by EC and identified several additional genes being co-amplified with the HER-2/neu gene. Comparative Analysis of RNA-based quantitative RT-PCR (TaqMan) with DNA-based qPCR from tumor cell lines identified the same amplified region. Genes at the 17q11.2-21. region are offered by way of illustration not by way of limitation. A graphical display of the described chromosomal region is provided in FIG. 1.

### Biological Relevance of the Genes which are Part of the 17q21 ARCHEON

### MLN50

By differential screening of cDNAs from breast cancer-derived metastatic axillary lymph nodes, TRAF4 and 3 other novel genes (MLN51, MLN62, MLN64) were identified that are overexpressed in breast cancer [Tomasetto et al., 1995, (3)]. One gene, which they designated MLN50, was mapped to 17q11-q21.3 by radioactive in situ hybridization. In breast cancer cell lines, overexpression of the 4 kb MLN50 mRNA was correlated with amplification of the gene and with amplification and overexpression of ERBB2, which maps to the same region. The authors suggested that the 2 genes belong to the same amplicon. Amplification of chromosomal region 17q11-q21 is one of the most common events occurring in human breast cancers. They reported that the predicted 261-amino acid MLN50 protein contains an N-terminal LIM domain and a C-terminal SH3 domain. They renamed the protein LASP1, for ‘LIM and SH3 protein.’ Northern blot analysis revealed that LASP1 mRNA was expressed at a basal level in all normal tissues examined and overexpressed in 8% of primary breast cancers. In most of these cancers, LASP1 and ERBB2 were simultaneously overexpressed.

### MLLT6

The MLLT6 (AF17) gene encodes a protein of 1,093 amino acids, containing a leucine-zipper dimerization motif located 3-prime of the fusion point and a cysteine-rich domain at the end terminus. AF17 was found to contain stretches of amino acids previously associated with domains involved in transcriptional repression or activation.

Chromosome translocations involving band 11q23 are associated with approximately 10% of patients with acute lymphoblastic leukemia (ALL) and more than 5% of patients with acute myeloid leukemia (AML). The gene at 11q23 involved in the translocations is variously designated ALL1, HRX, MLL, and TRX1. The partner gene in one of the rarer translocations, t(11;17)(q23;q21), designated MLLT6 on 17q12.

### ZNF144 (Mel18)

Mel18 cDNA encodes a novel cys-rich zinc finger motif. The gene is expressed strongly in most tumor cell lines, but its normal tissue expression was limited to cells of neural origin and was especially abundant in fetal neural cells. It belongs to a RING-finger motif family which includes BMI1. The MEL18/BMI1 gene family represents a mammalian homolog of the Drosophila ‘polycomb’ gene group, thereby belonging to a memory mechanism involved in maintaining the expression pattern of key regulatory factors such as Hox genes. Bmi1, Mel18 and M33 genes, as representative examples of mouse Pc-G genes. Common phenotypes observed in knockout mice mutant for each of these genes indicate an important role for Pc-G genes not only in regulation of Hox gene expression and axial skeleton development but also in control of proliferation and survival of haematopoietic cell lineages. This is in line with the observed proliferative deregulation observed in lymphoblastic leukemia. The MEL18 gene is conserved among vertebrates. Its mRNA is expressed at high levels in placenta, lung, and kidney, and at lower levels in liver, pancreas, and skeletal muscle. Interestingly, cervical and lumbo-sacral-HOX gene expression is altered in several primary breast cancers with respect to normal breast tissue with the HoxB gene cluster being present on 17q distal to the 17q21 locus. Moreover, delay of differentiation with persistent nests of proliferating cells was found in endothelial cells cocultured with HOXB7-transduced SkBr3 cells, which exhibit a 17q21 amplification. Tumorigenicity of these cells has been evaluated in vivo. Xenograft in athymic nude mice showed that SkBr3/HOXB7 cells developed tumors with an increased number of blood vessels, either irradiated or not, whereas parental SkBr3 cells did not show any tumor take unless mice were sublethally irradiated. As part of this invention, we have found MEL18 to be overexpressed specifically in tumors bearing Her-2/neu gene amplification, which can be critical for Hox expression.

Phosphatidylinositol-4-Phosphate 5-Kinase, Type II, Beta: PIP5K2B

Phosphoinositide kinases play central roles in signal transduction. Phosphatidylinositol-4-phosphate 5-kinases (PIP5Ks) phosphorylate phosphatidylinositol 4-phosphate, giving rise to phosphatidylinositol 4,5-bisphosphate. The PIP5K enzymes exist as multiple isoforms that have various immunoreactivities, kinetic properties, and molecular masses. They are unique in that they possess almost no homology to the kinase motifs present in other phosphatidylinositol, protein, and lipid kinases. By screening a human fetal brain cDNA library with the PIP5K2B EST the full length gene could be isolated. The deduced 416-amino acid protein is 78% identical to PIP5K2A. Using SDS-PAGE, the authors estimated that bacterially expressed PIP5K2B has a molecular mass of 47 kD. Northern blot analysis detected a 6.3-kb PIP5K2B transcript which was abundantly expressed in several human tissues. PIP5K2B interacts specifically with the juxtamembrane region of the p55 TNF receptor (TNFR1) and PIP5K2B activity is increased in mammalian cells by treatment with TNF-alpha. A modeled complex with membrane-bound substrate and ATP shows how a phosphoinositide kinase can phosphorylate its substrate in situ at the membrane interface. The substrate-binding site is open on 1 side, consistent with dual specificity for phosphatidylinositol 3- and 5-phosphates. Although the amino acid sequence of PIP5K2A does not show homology to known kinases, recombinant PIP5K2A exhibited kinase activity. PIP5K2A contains a putative Src homology 3 (SH3) domain-binding sequence. Overexpression of mouse PIP5K1B in COS7 cells induced an increase in short actin fibers and a decrease in actin stress fibers.

### TEM7

Using serial analysis of gene expression (SAGE) a partial cDNAs corresponding to several tumor endothelial markers (TEMs) that displayed elevated expression during tumor angiogenesis could be identified. Among the genes identified was TEM7. Using database searches and 5-prime RACE the entire TEM7 coding region, which encodes a 500-amino acid type I transmembrane protein has been described. The extracellular region of TEM7 contains a plexin-like domain and has weak homology to the ECM protein nidogen. The function of these domains, which are usually found in secreted and extracellular matrix molecules, is unknown. Nidogen itself belongs to the entactin protein family and helps to determine pathways of migrating axons by switching from circumferential to longitudinal migration. Entactin is involved in cell migration, as it promotes trophoblast outgrowth through a mechanism mediated by the RGD recognition site, and plays an important role during invasion of the endometrial basement membrane at implantation. As entactin promotes thymocyte adhesion but affects thymocyte migration only marginally, it is suggested that entactin may plays a role in thymocyte localization during T cell development.

In situ hybridization analysis of human colorectal cancer demonstrated that TEM7 was expressed clearly in the endothelial cells of the tumor stroma but not in the endothelial cells of normal colonic tissue. Using in situ hybridization to assay expression in various normal adult mouse tissues, they observed that TEM7 was largely undetectable in mouse tissues or tumors, but was abundantly expressed in mouse brain.

### ZNFN1A3

By screening a B-cell cDNA library with a mouse Aiolos N-terminal cDNA probe, a cDNA encoding human Aiolos, or ZNFN1A3, was obtained. The deduced 509-amino acid protein, which is 86% identical to its mouse counterpart, has 4 DNA-binding zinc fingers in its N terminus and 2 zinc fingers that mediate protein dimerization in its C terminus. These domains are 100% and 96% homologous to the corresponding domains in the mouse protein, respectively. Northern blot analysis revealed strong expression of a major 11.0- and a minor 4.4-kb ZNFN1A3 transcript in peripheral blood leukocytes, spleen, and thymus, with lower expression in liver, small intestine, and lung.

Ikaros (ZNFN1A1), a hemopoietic zinc finger DNA-binding protein, is a central regulator of lymphoid differentiation and is implicated in leukemogenesis. The execution of normal function of Ikaros requires sequence-specific DNA binding, transactivation, and dimerization domains. Mice with a mutation in a related zinc finger protein, Aiolos, are prone to B-cell lymphoma. In chemically induced murine lymphomas allelic losses on markers surrounding the Znfn1a1 gene were detected in 27% of the tumors analyzed. Moreover specific Ikaros expression was in primary mouse hormone-producing anterior pituitary cells and substantial for Fibroblast growth factor receptor 4 (FGFR4) expression, which itself is implicated in a multitude of endocrine cell hormonal and proliferative properties with FGFR4 being differentially expressed in normal and neoplastic pituitary. Moreover Ikaros binds to chromatin remodelling complexes containing SWI/SNF proteins, which antagonize Polycomb function. Interestingly at the telomeric end of the disclosed ARCHEON the SWI/SNF complex member SMARCE1 (=SWI/SNF-related, matrix-associated, actin-dependent regulators of chromatin) is located and part of the described amplification. Due to the related binding specificities of Ikaros and Palindrom Binding Protein (PBP) it is suggestive, that ZNFN1A3 is able to regulate the Her-2/neu enhancer.

### PPP1R1B

Midbrain dopaminergic neurons play a critical role in multiple brain functions, and abnormal signaling through dopaminergic pathways has been implicated in several major neurologic and psychiatric disorders. One well-studied target for the actions of dopamine is DARPP32. In the densely dopamine- and glutamate-innervated rat caudate-putamen, DARPP32 is expressed in medium-sized spiny neurons that also express dopamine D1 receptors. The function of DARPP32 seems to be regulated by receptor stimulation. Both dopaminergic and glutamatergic (NMDA) receptor stimulation regulate the extent of DARPP32 phosphorylation, but in opposite directions.

The human DARPP32 was isolated from a striatal cDNA library. The 204-amino acid DARPP32 protein shares 88% and 85% sequence identity, respectively, with bovine and rat DARPP32 proteins. The DARPP32 sequence is particularly conserved through the N terminus, which represents the active portion of the protein. Northern blot analysis demonstrated that the 2.1-kb DARPP32 mRNA is more highly expressed in human caudate than in cortex. In situ hybridization to postmortem human brain showed a low level of DARPP32 expression in all neocortical layers, with the strongest hybridization in the superficial layers. CDK5 phosphorylated DARPP32 in vitro and in intact brain cells. Phospho-thr75 DARPP32 inhibits PKA in vitro by a competitive mechanism. Decreasing phospho-thr75 DARPP32 in striatal cells either by a CDK5-specific inhibitor or by using genetically altered mice resulted in increased dopamine-induced phosphorylation of PKA substrates and augmented peak voltage-gated calcium currents. Thus, DARPP32 is a bifunctional signal transduction molecule which, by distinct mechanisms, controls a serine/threonine kinase and a serine/threonine phosphatase.

DARPP32 and t-DARPP are overexpressed in gastric cancers. It's suggested that overexpression of these 2 proteins in gastric cancers may provide an important survival advantage to neoplastic cells. It could be demonstrated that Darpp32 is an obligate intermediate in progesterone-facilitated sexual receptivity in female rats and mice. The facilitative effect of progesterone on sexual receptivity in female rats was blocked by antisense oligonucleotides to Darpp32. Homozygous mice carrying a null mutation for the Darpp32 gene exhibited minimal levels of progesterone-facilitated sexual receptivity when compared to their wildtype littermates, and progesterone significantly increased hypothalamic cAMP levels and cAMP-dependent protein kinase activity.

### CACNB1

In 1991 a cDNA clone encoding a protein with high homology to the beta subunit of the rabbit skeletal muscle dihydropyridine-sensitive calcium channel from a rat brain cDNA library [Pragnell et al., 1991, (4)]. This rat brain beta-subunit cDNA hybridized to a 3.4-kb message that was expressed in high levels in the cerebral hemispheres and hippocampus and much lower levels in cerebellum. The open reading frame encodes 597 amino acids with a predicted mass of 65,679 Da which is 82% homologous with the skeletal muscle beta subunit. The corresponding human beta-subunit gene was localized to chromosome 17 by analysis of somatic cell hybrids. The authors suggested that the encoded brain beta subunit, which has a primary structure highly similar to its isoform in skeletal muscle, may have a comparable role as an integral regulatory component of a neuronal calcium channel.

### RPL19

The ribosome is the only organelle conserved between prokaryotes and eukaryotes. In eukaryotes, this organelle consists of a 60S large subunit and a 40S small subunit. The mammalian ribosome contains 4 species of RNA and approximately 80 different ribosomal proteins, most of which appear to be present in equimolar amounts. In mammalian cells, ribosomal proteins can account for up to 15% of the total cellular protein, and the expression of the different ribosomal protein genes, which can account for up to 7 to 9% of the total cellular mRNAs, is coordinately regulated to meet the cell's varying requirements for protein synthesis. The mammalian ribosomal protein genes are members of multigene families, most of which are composed of multiple processed pseudogenes and a single functional intron-containing gene. The presence of multiple pseudogenes hampered the isolation and study of the functional ribosomal protein genes. By study of somatic cell hybrids, it has been elucidated that DNA sequences complementary to 6 mammalian ribosomal protein cDNAs could be assigned to chromosomes 5, 8, and 17. Ten fragments mapped to 3 chromosomes [Nakamichi et al., 1986, (5)]. These are probably a mixture of functional (expressed) genes and pseudogenes. One that maps to 5q23-q33 rescues Chinese hamster emetine-resistance mutations in interspecies hybrids and is therefore the transcriptionally active RPS14 gene. In 1989 a PCR-based strategy for the detection of intron-containing genes in the presence of multiple pseudogenes was described. This technique was used to identify the intron-containing PCR products of 7 human ribosomal protein genes and to map their chromosomal locations by hybridization to human/rodent somatic cell hybrids [Feo et al., 1992, (6)]. All 7 ribosomal protein genes were found to be on different chromosomes: RPL19 on 17p12-q11; RPL30 on 8; RPL35A on 18; RPL36A on 14; RPS6 on 9pter-p13; RPS11 on 19cen-qter; and RPS17 on 11pter-p13. These are also different sites from the chromosomal location of previously mapped ribosomal protein genes S14 on chromosome 5, S4 on Xq and Yp, and RP117A on 9q3-q34. By fluorescence in situ hybridization the position of the RPL19 gene was mapped to 17q11 [Davies et al., 1989, (7)].

### PPARBP, PBP, CRSP1, CRSP200, TRIP2, TRAP220, RB18A, DRIP230

The thyroid hormone receptors (TRs) are hormone-dependent transcription factors that regulate expression of a variety of specific target genes. They must specifically interact with a number of proteins as they progress from their initial translation and nuclear translocation to heterodimerization with retinoid X receptors (RXRs), functional interactions with other transcription factors and the basic transcriptional apparatus, and eventually, degradation. To help elucidate the mechanisms that underlie the transcriptional effects and other potential functions of TRs, the yeast interaction trap, a version of the yeast 2-hybrid system, was used to identify proteins that specifically interact with the ligand-binding domain of rat TR-beta-1 (THRB) [Lee et al., 1995, (8)]. The authors isolated HeLa cell cDNAs encoding several different TR-interacting proteins (TRIPs), including TRIP2. TRIP2 interacted with rat Thrb only in the presence of thyroid hormone. It showed a ligand-independent interaction with RXR-alpha, but did not interact with the glucocorticoid receptor (NR3C1) under any condition. By immunoscreening a human B-lymphoma cell cDNA expression library with the anti-p53 monoclonal antibody PAb1801, PPARBP was identified, which was called RB18A for ‘recognized by PAb1801 monoclonal antibody’ [Drane et al., 1997, (9)]. The predicted 1,566-amino acid RB18A protein contains several potential nuclear localization signals, 13 potential N-glycosylation sites, and a high number of potential phosphorylation sites. Despite sharing common antigenic determinants with p53, RB18A does not show significant nucleotide or amino acid sequence similarity with p53. Whereas the calculated molecular mass of RB18A is 166 kD, the apparent mass of recombinant RB18A was 205 kD by SDS-PAGE analysis. The authors demonstrated that RB18A shares functional properties with p53, including DNA binding, p53 binding, and self-oligomerization. Furthermore, RB18A was able to activate the sequence-specific binding of p53 to DNA, which was induced through an unstable interaction between both proteins. Northern blot analysis of human tissues detected an 8.5-kb RB18A transcript in all tissues examined except kidney, with highest expression in heart. Moreover mouse Pparbp, which was called Pbp for ‘Ppar-binding protein,’ as a protein that interacts with the Ppar-gamma (PPARG) ligand-binding domain in a yeast 2-hybrid system was identified [Zhu et al., 1997, (10)]. The authors found that Pbp also binds to PPAR-alpha (PPARA), RAR-alpha (RARA), RXR, and TR-beta-1 in vitro. The binding of Pbp to these receptors increased in the presence of specific ligands. Deletion of the last 12 amino acids from the C terminus of PPAR-gamma resulted in the abolition of interaction between Pbp and PPAR-gamma. Pbp modestly increased the transcriptional activity of PPAR-gamma, and a truncated form of Pbp acted as a dominant-negative repressor, suggesting that Pbp is a genuine transcriptional co-activator for PPAR. The predicted 1,560-amino acid Pbp protein contains 2 LXXLL motifs, which are considered necessary and sufficient for the binding of several co-activators to nuclear receptors. Northern blot analysis detected Pbp expression in all mouse tissues examined, with higher levels in liver, kidney, lung, and testis. In situ hybridization showed that Pbp is expressed during mouse ontogeny, suggesting a possible role for Pbp in cellular proliferation and differentiation. In adult mouse, in situ hybridization detected Pbp expression in liver, bronchial epithelium in the lung, intestinal mucosa, kidney cortex, thymic cortex, splenic follicles, and seminiferous epithelium in testis. Lateron PPARBP was identified, which was called TRAP220, from an immunopurified TR-alpha (THRA)-TRAP complex [Yuan et al., 1998, (11)]. The authors cloned Jurkat cell cDNAs encoding TRAP220. The predicted 1,581-amino acid TRAP220 protein contains LXXLL domains, which are found in other nuclear receptor-interacting proteins. TRAP220 is nearly identical to RB18A, with these proteins differing primarily by an extended N terminus on TRAP220. In the absence of TR-alpha, TRAP220 appears to reside in a single complex with other TRAPs. TRAP220 showed a direct ligand-dependent interaction with TR-alpha, which was mediated through the C terminus of TR-alpha and, at least in part, the LXXLL domains of TRAP220. TRAP220 also interacted with other nuclear receptors, including vitamin D receptor, RARA, RXRA, PPARA, PPARG, and estrogen receptor-alpha (ESR1; 133430), in a ligand-dependent manner. TRAP220 moderately stimulated human TR-alpha-mediated transcription in transfected cells, whereas a fragment containing the LXXLL motifs acted as a dominant-negative inhibitor of nuclear receptor-mediated transcription both in transfected cells and in cell-free transcription systems. Further studies indicated that TRAP220 plays a major role in anchoring other TRAPs to TR-alpha during the function of the TR-alpha-TRAP complex and that TRAP220 may be a global co-activator for the nuclear receptor superfamily. PBP, a nuclear receptor co-activator, interacts with estrogen receptor-alpha (ESR1) in the absence of estrogen. This interaction was enhanced in the presence of estrogen, but was reduced in the presence of the anti-estrogen Tamoxifen. Transfection of PBP into cultured cells resulted in enhancement of estrogen-dependent transcription, indicating that PBP serves as a co-activator in estrogen receptor signaling. To examine whether overexpression of PBP plays a role in breast cancer because of its co-activator function in estrogen receptor signaling, the levels of PBP expression in breast tumors was determined [Zhu et al., 1999, (12)]. High levels of PBP expression were detected in approximately 50% of primary breast cancers and breast cancer cell lines by ribonuclease protection analysis, in situ hybridization, and immunoperoxidase staining. By using FISH, the authors mapped the PBP gene to 17q12, a region that is amplified in some breast cancers. They found PBP gene amplification in approximately 24% (6 of 25) of breast tumors and approximately 30% (2 of 6) of breast cancer cell lines, implying that PBP gene overexpression can occur independent of gene amplification. They determined that the PBP gene comprises 17 exons that together span more than 37 kb. Their findings, in particular PBP gene amplification, suggested that PBP, by its ability to function as an estrogen receptor-alpha co-activator, may play a role in mammary epithelial differentiation and in breast carcinogenesis.

### NEUROD2

Basic helix-loop-helix (bHLH) proteins are transcription factors involved in determining cell type during development. In 1995 a bHLH protein was described, termed NeuroD (for ‘neurogenic differentiation’), that functions during neurogenesis [Lee et al., 1995, (13)]. The human NEUROD gene maps to chromosome 2q32. The cloning and characterization of 2 additional NEUROD genes, NEUROD2 and NEUROD3 was described in 1996 [McCormick et al., 1996, (14)]. Sequences for the mouse and human homologues were presented. NEUROD2 shows a high degree of homology to the bHLH region of NEUROD, whereas NEUROD3 is more distantly related. The authors found that mouse neuroD2 was initially expressed at embryonic day 11, with persistent expression in the adult nervous system. Similar to neuroD, neuroD2 appears to mediate neuronal differentiation. The human NEUROD2 was mapped to 17q12 by fluorescence in situ hybridization and the mouse homologue to chromosome 11 [Tamimi et al., 1997, (15)].

### Telethonin

Telethonin is a sarcomeric protein of 19 kD found exclusively in striated and cardiac muscle It appears to be localized to the Z disc of adult skeletal muscle and cultured myocytes. Telethonin is a substrate of titin, which acts as a molecular ‘ruler’ for the assembly of the sarcomere by providing spatially defined binding sites for other sarcomeric proteins. After activation by phosphorylation and calcium/calmodulin binding, titin phosphorylates the C-terminal domain of telethonin in early differentiating myocytes. The telethonin gene has been mapped to 17q12, adjacent to the phenylethanolamine N-methyltransferase gene [Valle et al., 1997, (16)].

### PENT, PNMT

Phenylethanolamine N-methyltransferase catalyzes the synthesis of epinephrine from norepinephrine, the last step of catecholamine biosynthesis. The cDNA clone was first isolated in 1998 for bovine adrenal medulla PNMT using mixed oligodeoxyribonucleotide probes whose synthesis was based on the partial amino acid sequence of tryptic peptides from the bovine enzyme [Kaneda et al., 1988, (17)]. Using a bovine cDNA as a probe, the authors screened a human pheochromocytoma cDNA library and isolated a cDNA clone with an insert of about 1.0 kb, which contained a complete coding region of the enzyme. Northern blot analysis of human pheochromocytoma polyadenylated RNA using this cDNA insert as the probe demonstrated a single RNA species of about 1,000 nucleotides, suggesting that this clone is a full-length cDNA. The nucleotide sequence showed that human PNMT has 282 amino acid residues with a predicted molecular weight of 30,853, including the initial methionine. The amino acid sequence was 88% homologous to that of bovine enzyme. The PNMT gene was found to consist of 3 exons and 2 introns spanning about 2,100 basepairs. It was demonstrated that in transgenic mice the gene is expressed in adrenal medulla and retina. A hybrid gene consisting of 2 kb of the PNMT 5-prime-flanking region fused to the simian virus 40 early region also resulted in tumor antigen mRNA expression in adrenal glands and eyes; furthermore, immunocytochemistry showed that the tumor antigen was localized in nuclei of adrenal medullary cells and cells of the inner nuclear cell layer of the retina, both prominent sites of epinephrine synthesis. The results indicate that the enhancer(s) for appropriate expression of the gene in these cell types are in the 2-kb 5-prime-flanking region of the gene.

Kaneda et al., 1988 (17), assigned the human PNMT gene to chromosome 17 by Southern blot analysis of DNA from mouse-human somatic cell hybrids. In 1992 the localization was narrowed down to 17q21-q22 by linkage analysis using RFLPs related to the PNMT gene and several 17q DNA markers [Hoehe et al., 1992, (18)]. The findings are of interest in light of the description of a genetic locus associated with blood pressure regulation in the stroke-prone spontaneously hypertensive rat (SHR-SP) on rat chromosome 10 in a conserved linkage synteny group corresponding to human chromosome 17q22-q24. See essential hypertension.

### MGC9753

This gene maps on chromosome 17, at 17q12 according to RefSeq. It is expressed at very high level. It is defined by cDNA clones and produces, by alternative splicing, 7 different transcripts can be obtained (SEQ ID NO:60 to 66 and 83 to 89, Table 1), altogether encoding 7 different protein isoforms. Of specific interest is the putatively secreted isoform g, encoded by a mRNA of 2.55 kb. It's premessenger covers 16.94 kb on the genome. It has a very long 3′ UTR. The protein (226 aa, MW 24.6 kDa, pI 8.5) contains no Pfam motif. The MGC9753 gene produces, by alternative splicing, 7 types of transcripts, predicted to encode 7 distinct proteins. It contains 13 confirmed introns, 10 of which are alternative. Comparison to the genome sequence shows that 11 introns follow the consensual [gt-ag] rule, 1 is atypical with good support [tg_cg]. The six most abundant isoforms are designated by a) to i) and code for proteins as follows:
a) This mRNA is 3.03 kb long, its premessenger covers 16.95 kb on the genome. It has a very long 3′ UTR. The protein (190 aa, MW 21.5 kDa, pI 7.2) contains no Pfam motif. It is predicted to localise in the endoplasmic reticulum.
c) This mRNA is 1.17 kb long, its premessenger covers 16.93 kb on the genome. It may be incomplete at the N terminus. The protein (368 aa, MW 41.5 kDa, pI 7.3) contains no Pfam motif.
d) This mRNA is 3.17 kb long, its premessenger covers 16.94 kb on the genome. It has a very long 3′ UTR and 5′p UTR. The protein (190 aa, MW 21.5 kDa, pI 7.2) contains no Pfam motif. It is predicted to localise in the endoplasmic reticulum.
g) This mRNA is 2.55 kb long, its premessenger covers 16.94 kb on the genome. It has a very long 3′ UTR. The protein (226 aa, MW 24.6kDa, pI 8.5) contains no Pfam motif. It is predicted to be secreted.
h) This mRNA is 2.68 kb long, its premessenger covers 16.94 kb on the genome. It has a very long 3′ UTR. The protein (320 aa, MW 36.5 kDa, pI 6.8) contains no Pfam motif. It is predicted to localise in the endoplasmic reticulum.
i) This mRNA is 2.34 kb long, its premessenger covers 16.94 kb on the genome. It may be incomplete at the N terminus. It has a very long 3′ UTR. The protein (217 aa, MW 24.4 kDa, pI 5.9) contains no Pfam motif.

The MCG9753 gene may be homologue to the CAB2 gene located on chromosome 17q12. The CAB2, a human homologue of the yeast COS 16 required for the repair of DNA double-strand breaks was cloned. Autofluorescence analysis of cells transfected with its GFP fusion protein demonstrated that CAB2 translocates into vesicles, suggesting that overexpression of CAB2 may decrease intercellular Mn—

(2+) by accumulating it in the vesicles, in the same way as yeast.

### Her-2/neu, ERBB2, NGL, TKR1

The oncogene originally called NEU was derived from rat neuro/glioblastoma cell lines. It encodes a tumor antigen, p185, which is serologically related to EGFR, the epidermal growth factor receptor. EGFR maps to chromosome 7. In 1985 it was found, that the human homologue, which they designated NGL (to avoid confusion with neuraminidase, which is also symbolized NEU), maps to 17q12-q22 by in situ hybridization and to 17q21-qter in somatic cell hybrids [Yang-Feng et al., 1985, (19)]. Thus, the SRO is 17q21-q22. Moreover, in 1985 a potential cell surface receptor of the tyrosine kinase gene family was identified and characterized by cloning the gene [Coussens et al., 1985, (20)]. Its primary sequence is very similar to that of the human epidermal growth factor receptor. Because of the seemingly close relationship to the human EGF receptor, the authors called the gene HER2. By Southern blot analysis of somatic cell hybrid DNA and by in situ hybridization, the gene was assigned to 17q21-q22. This chromosomal location of the gene is coincident with the NEU oncogene, which suggests that the 2 genes may in fact be the same; indeed, sequencing indicates that they are identical. In 1988 a correlation between overexpression of NEU protein and the large-cell, comedo growth type of ductal carcinoma was found [van de Vijver et al., 1988, (21)]. The authors found no correlation, however, with lymph-node status or tumor recurrence. The role of HER2/NEU in breast and ovarian cancer was described in 1989, which together account for one-third of all cancers in women and approximately one-quarter of cancer-related deaths in females [Slamon et al., 1989, (22)].

An ERBB-related gene that is distinct from the ERBB gene, called ERBB1 was found in 1985. ERBB2 was not amplified in vulva carcinoma cells with EGFR amplification and did not react with EGF receptor mRNA. About 30-fold amplification of ERBB2 was observed in a human adenocarcinoma of the salivary gland. By chromosome sorting combined with velocity sedimentation and Southern hybridization, the ERBB2 gene was assigned to chromosome 17 [Fukushige et al., 1986, (23)]. By hybridization to sorted chromosomes and to metaphase spreads with a genomic probe, they mapped the ERBB2 locus to 17q21. This is the chromosome 17 breakpoint in acute promyelocytic leukemia (APL). Furthermore, they observed amplification and elevated expression of the ERBB2 gene in a gastric cancer cell line. Antibodies against a synthetic peptide corresponding to 14 amino acid residues at the COOH-terminus of a protein deduced from the ERBB2 nucleotide sequence were raised in 1986. With these antibodies, the ERBB2 gene product from adenocarcinoma cells was precipitated and demonstrated to be a 185-kD glycoprotein with tyrosine kinase activity. A cDNA probe for ERBB2 and by in situ hybridization to APL cells with a 15;17 chromosome translocation located the gene to the proximal side of the breakpoint [Kaneko et al., 1987, (24)]. The authors suggested that both the gene and the breakpoint are located in band 17q21.1 and, further, that the ERBB2 gene is involved in the development of leukemia. In 1987 experiments indicated that NEU and HER2 are both the same as ERBB2 [Di Fiore et al., 1987, (25)]. The authors demonstrated that overexpression alone can convert the gene for a normal growth factor receptor, namely, ERBB2, into an oncogene. The ERBB2 to 17q11-q21 by in situ hybridization [Popescu et al., 1989, (26)]. By in situ hybridization to chromosomes derived from fibroblasts carrying a constitutional translocation between 15 and 17, they showed that the ERBB2 gene was relocated to the derivative chromosome 15; the gene can thus be localized to 17q12-q21.32. By family linkage studies using multiple DNA markers in the 17q12-q21 region the ERBB2 gene was placed on the genetic map of the region.

Interleukin-6 is a cytokine that was initially recognized as a regulator of immune and inflammatory responses, but also regulates the growth of many tumor cells, including prostate cancer. Overexpression of ERBB2 and ERBB3 has been implicated in the neoplastic transformation of prostate cancer. Treatment of a prostate cancer cell line with IL6 induced tyrosine phosphorylation of ERBB2 and ERBB3, but not ERBB1/EGFR. The ERBB2 forms a complex with the gp130 subunit of the IL6 receptor in an IL6-dependent manner. This association was important because the inhibition of ERBB2 activity resulted in abrogation of IL6-induced MAPK activation. Thus, ERBB2 is a critical component of IL6 signaling through the MAP kinase pathway [Qiu et al., 1998, (27)]. These findings showed how a cytokine receptor can diversify its signaling pathways by engaging with a growth factor receptor kinase.

Overexpression of ERBB2 confers Taxol resistance in breast cancers. Overexpression of ERBB2 inhibits Taxol-induced apoptosis [Yu et al., 1998, (28)]. Taxol activates CDC2 kinase in MDA-MB-435 breast cancer cells, leading to cell cycle arrest at the G2/M phase and, subsequently, apoptosis. A chemical inhibitor of CDC2 and a dominant-negative mutant of CDC2 blocked Taxol-induced apoptosis in these cells. Overexpression of ERBB2 in MDA-MB435 cells by transfection transcriptionally upregulates CDKN1A which associates with CDC2, inhibits Taxol-mediated CDC2 activation, delays cell entrance to G2/M phase, and thereby inhibits Taxol-induced apoptosis. In CDKN1A antisense-transfected MDA-MB-435 cells or in p21−/− MEF cells, ERBB2 was unable to inhibit Taxol-induced apoptosis. Therefore, CDKN1A participates in the regulation of a G2/M checkpoint that contributes to resistance to Taxol-induced apoptosis in ERBB2-overexpressing breast cancer cells.

A secreted protein of approximately 68 kD was described, designated herstatin, as the product of an alternative ERBB2 transcript that retains intron 8 [Doherty et al., 1999, (29)]. This alternative transcript specifies 340 residues identical to subdomains I and II from the extracellular domain of p185ERBB2, followed by a unique C-terminal sequence of 79 amino acids encoded by intron 8. The recombinant product of the alternative transcript specifically bound to ERBB2-transfected cells and was chemically crosslinked to p185ERBB2, whereas the intron-encoded sequence alone also bound with high affinity to transfected cells and associated with p185 solubilized from cell extracts. The herstatin mRNA was expressed in normal human fetal kidney and liver, but was at reduced levels relative to p185ERBB2 mRNA in carcinoma cells that contained an amplified ERBB2 gene. Herstatin appears to be an inhibitor of p185ERBB2, because it disrupts dimers, reduces tyrosine phosphorylation of p185, and inhibits the anchorage-independent growth of transformed cells that overexpress ERBB2. The HER2 gene is amplified and HER2 is overexpressed in 25 to 30% of breast cancers, increasing the aggressiveness of the tumor. Finally, it was found that a recombinant monoclonal antibody against HER2 increased the clinical benefit of first-line chemotherapy in metastatic breast cancer that overexpresses HER2 [Slamon et al., 2001, (30)].

### GRB7

Growth factor receptor tyrosine kinases (GF-RTKs) are involved in activating the cell cycle. Several substrates of GF-RTKs contain Src-homology 2 (SH2) and SH3 domains. SH2 domain-containing proteins are a diverse group of molecules important in tyrosine kinase signaling. Using the CORT (cloning of receptor targets) method to screen a high expression mouse library, the gene for murine Grb7, which encodes a protein of 535 amino acids, was isolated [Margolis et al., 1992, (31)]. GRB7 is homologous to ras-GAP (ras-GTPase-activating protein). It contains an SH2 domain and is highly expressed in liver and kidney. This gene defines the GRB7 family, whose members include the mouse gene Grb10 and the human gene GRB14.

A putative GRB7 signal transduction molecule and a GRB7V novel splice variant from an invasive human esophageal carcinoma was isolated [Tanaka et al., 1998, (32)]. Although both GRB7 isoforms shared homology with the Mig-10 cell migration gene of Caenorhabditis elegans, the GRB7V isoform lacked 88 basepairs in the C terminus; the resultant frameshift led to substitution of an SH2 domain with a short hydrophobic sequence. The wildtype GRB7 protein, but not the GRB7V isoform, was rapidly tyrosyl phosphorylated in response to EGF stimulation in esophageal carcinoma cells. Analysis of human esophageal tumor tissues and regional lymph nodes with metastases revealed that GRB7V was expressed in 40% of GRB7-positive esophageal carcinomas. GRB7V expression was enhanced after metastatic spread to lymph nodes as compared to the original tumor tissues. Transfection of an antisense GRB7 RNA expression construct lowered endogenous GRB7 protein levels and suppressed the invasive phenotype exhibited by esophageal carcinoma cells. These findings suggested that GRB7 isoforms are involved in cell invasion and metastatic progression of human esophageal carcinomas. By sequence analysis, The GRB7 gene was mapped to chromosome 17q2122, near the topoisomerase-2 gene [Dong et al., 1997, (33)]. GRB-7 is amplified in concert with HER2 in several breast cancer cell lines and that GRB-7 is overexpressed in both cell lines and breast tumors. GRB-7, through its SH2 domain, binds tightly to HER2 such that a large fraction of the tyrosine phosphorylated HER2 in SKBR-3 cells is bound to GRB-7 [Stein et al., 1994, (34)].

### GCSF, CSF3

Granulocyte colony-stimulating factor (or colony stimulating factor-3) specifically stimulates the proliferation and differentiation of the progenitor cells for granulocytes. The partial amino acid sequence of purified GCSF protein was determined, and by using oligonucleotides as probes, several GCSF cDNA clones were isolated from a human squamous carcinoma cell line cDNA library [Nagata et al., 1986, (35)]. Cloning of human GCSF cDNA shows that a single gene codes for a 177- or 180-amino acid mature protein of molecular weight 19,600. The authors found that the GCSF gene has 4 introns and that 2 different polypeptides are synthesized from the same gene by differential splicing of mRNA. The 2 polypeptides differ by the presence or absence of 3 amino acids. Expression studies indicate that both have authentic GCSF activity. A stimulatory activity from a glioblastoma multiform cell line being biologically and biochemically indistinguishable from GCSF produced by a bladder cell line was found in 1987. By somatic cell hybridization and in situ chromosomal hybridization, the GCSF gene was mapped to 17q11 in the region of the breakpoint in the 15;17 translocation characteristic of acute promyelocytic leukemia [Le Beau et al., 1987, (36)]. Further studies indicated that the gene is proximal to the said breakpoint and that it remains on the rearranged chromosome 17. Southern blot analysis using both conventional and pulsed field gel electrophoresis showed no rearranged restriction fragments. By use of a full-length cDNA clone as a hybridization probe in human-mouse somatic cell hybrids and in flow-sorted human chromosomes, the gene for GCSF was mapped to 17q21-q22 lateron

### THRA, THRA1, ERBA, EAR7, ERBA2, ERBA3

Both human and mouse DNA have been demonstrated to have two distantly related classes of ERBA genes and that in the human genome multiple copies of one of the classes exist [Jansson et al., 1983, (37)]. A cDNA was isolated derived from rat brain messenger RNA on the basis of homology to the human thyroid receptor gene [Thompson et al., 1987, (38)]. Expression of this cDNA produced a high-affinity binding protein for thyroid hormones. Messenger RNA from this gene was expressed in tissue-specific fashion, with highest levels in the central nervous system and no expression in the liver. An increasing body of evidence indicated the presence of multiple thyroid hormone receptors. The authors suggested that there may be as many as 5 different but related loci. Many of the clinical and physiologic studies suggested the existence of multiple receptors. For example, patients had been identified with familial thyroid hormone resistance in which peripheral response to thyroid hormones is lost or diminished while neuronal functions are maintained. Thyroidologists recognize a form of cretinism in which the nervous system is severely affected and another form in which the peripheral functions of thyroid hormone are more dramatically affected.

The cDNA encoding a specific form of thyroid hormone receptor expressed in human liver, kidney, placenta, and brain was isolated [Nakai et al., 1988, (39)]. Identical clones were found in human placenta. The cDNA encodes a protein of 490 amino acids and molecular mass of 54,824. Designated thyroid hormone receptor type alpha-2 (THR2), this protein is represented by mRNAs of different size in liver and kidney, which may represent tissue-specific processing of the primary transcript.

The THRA gene contains 10 exons spanning 27 kb of DNA. The last 2 exons of the gene are alternatively spliced. A 5-kb THRA1 mRNA encodes a predicted 410-amino acid protein; a 2.7-kb THRA2 mRNA encodes a 490-amino acid protein. A third isoform, TR-alpha-3, is derived by alternative splicing. The proximal 39 amino acids of the TH-alpha-2 specific sequences are deleted in TR-alpha-3. A second gene, THRB on chromosome 3, encodes 2 isoforms of TR-beta by alternative splicing. In 1989 the structure and function of the EAR1 and EAR7 genes was elucidated, both located on 17q21 [Miyajima et al., 1989, (40)]. The authors determined that one of the exons in the EAR7 coding sequence overlaps an exon of EAR1, and that the 2 genes are transcribed from opposite DNA strands. In addition, the EAR7 mRNA generates 2 alternatively spliced isoforms, referred to as EAR71 and EAR72, of which the EAR71 protein is the human counterpart of the chicken c-erbA protein.

The thyroid hormone receptors, beta, alpha-1, and alpha-2 3 mRNAs are expressed in all tissues examined and the relative amounts of the three mRNAs were roughly parallel. None of the 3 mRNAs was abundant in liver, which is the major thyroid hormone-responsive organ. This led to the assumption that another thyroid hormone receptor may be present in liver. It was found that ERBA, which potentiates ERBB, has an amino acid sequence different from that of other known oncogene products and related to those of the carbonic anhydrases [Debuire et al., 1984, (41)]. ERBA potentiates ERBB by blocking differentiation of erythroblasts at an immature stage. Carbonic anhydrases participate in the transport of carbon dioxide in erythrocytes. In 1986 it was shown that the ERBA protein is a high-affinity receptor for thyroid hormone. The cDNA sequence indicates a relationship to steroid-hormone receptors, and binding studies indicate that it is a receptor for thyroid hormones. It is located in the nucleus, where it binds to DNA and activates transcription.

Maternal thyroid hormone is transferred to the fetus early in pregnancy and is postulated to regulate brain development. The ontogeny of TR isoforms and related splice variants in 9 first-trimester fetal brains by semi-quantitative RT-PCR analysis has been investigated. Expression of the TR-beta-1, TR-alpha-1, and TR-alpha-2 isoforms was detected from 8.1 weeks' gestation. An additional truncated species was detected with the TR-alpha-2 primer set, consistent with the TK-alpha-3 splice variant described in the rat. All TR-alpha-derived transcripts were coordinately expressed and increased approximately 8-fold between 8.1 and 13.9 weeks' gestation. A more complex ontogenic pattern was observed for TR-beta-1, suggestive of a nadir between 8.4 and 12.0 weeks' gestation. The authors concluded that these findings point to an important role for the TR-alpha-1 isoform in mediating maternal thyroid hormone action during first-trimester fetal brain development.

The identification of the several types of thyroid hormone receptor may explain the normal variation in thyroid hormone responsiveness of various organs and the selective tissue abnormalities found in the thyroid hormone resistance syndromes. Members of sibships, who were resistant to thyroid hormone action, had retarded growth, congenital deafness, and abnormal bones, but had normal intellect and sexual maturation, as well as augmented cardiovascular activity. In this family abnormal T3 nuclear receptors in blood cells and fibroblasts have been demonstrated. The availability of cDNAs encoding the various thyroid hormone receptors was considered useful in determining the underlying genetic defect in this family.

The ERBA oncogene has been assigned to chromosome 17. The ERBA locus remains on chromosome 17 in the t(15;17) translocation of acute promyelocytic leukemia (APL). The thymidine kinase locus is probably translocated to chromosome 15; study of leukemia with t(17;21) and apparently identical breakpoint showed that TK was on 21q+. By in situ hybridization of a cloned DNA probe of c-erb-A to meiotic pachytene spreads obtained from uncultured spermatocytes it has been concluded that ERBA is situated at 17q21.33-17q22, in the same region as the break that generated the t(15;17) seen in APL. Because most of the grains were seen in 17q22, they suggested that ERBA is probably in the proximal region of 17q22 or at the junction between 17q22 and 17q21.33. By in situ hybridization it has been demonstrated, that that ERBA remains at 17q11-q12 in APL, whereas TP53, at 17q21-q22, is translocated to chromosome 15. Thus, ERBA must be at 17q11.2 just proximal to the breakpoint in the APL translocation and just distal to it in the constitutional translocation.

The aberrant THRA expression in nonfunctioning pituitary tumors has been hypothesized to reflect mutations in the receptor coding and regulatory sequences. They screened THRA mRNA and THRB response elements and ligand-binding domains for sequence anomalies. Screening THRA mRNA from 23 tumors by RNAse mismatch and sequencing candidate fragments identified 1 silent and 3 missense mutations, 2 in the common THRA region and 1 that was specific for the alpha-2 isoform. No THRB response element differences were detected in 14 nonfunctioning tumors, and no THRB ligand-binding domain differences were detected in 23 nonfunctioning tumors. Therefore it has been suggested that the novel thyroid receptor mutations may be of functional significance in terms of thyroid receptor action, and further definition of their functional properties may provide insight into the role of thyroid receptors in growth control in pituitary cells.

### RAR-alpha

A cDNA encoding a protein that binds retinoic acid with high affinity has been cloned [Petkovich et al., 1987, (42)]. The protein was found to be homologous to the receptors for steroid hormones, thyroid hormones, and vitamin D3, and appeared to be a retinoic acid-inducible transacting enhancer factor. Thus, the molecular mechanisms of the effect of vitamin A on embryonic development, differentiation and tumor cell growth may be similar to those described for other members of this nuclear receptor family. In general, the DNA-binding domain is most highly conserved, both within and between the 2 groups of receptors (steroid and thyroid); Using a cDNA probe, the RAR-alpha gene has been mapped to 17q21 by in situ hybridization [Mattei et al., 1988, (43)]. Evidence has been presented for the existence of 2 retinoic acid receptors, RAR-alpha and RAR-beta, mapping to chromosome 17q21.1 and 3p24, respectively. The alpha and beta forms of RAR were found to be more homologous to the 2 closely related thyroid hormone receptors alpha and beta, located on 17q11.2 and 3p25-p21, respectively, than to any other members of the nuclear receptor family. These observations suggest that the thyroid hormone and retinoic acid receptors evolved by gene, and possibly chromosome, duplications from a common ancestor, which itself diverged rather early in evolution from the common ancestor of the steroid receptor group of the family. They noted that the counterparts of the human RARA and RARB genes are present in both the mouse and chicken. The involvement of RARA at the APL breakpoint may explain why the use of retinoic acid as a therapeutic differentiation agent in the treatment of acute myeloid leukemias is limited to APL. Almost all patients with APL have a chromosomal translocation t(15;17)(q22;q21). Molecular studies reveal that the translocation results in a chimeric gene through fusion between the PML gene on chromosome 15 and the RARA gene on chromosome 17. A hormone-dependent interaction of the nuclear receptors RARA and RXRA with CLOCK and MOP4 has been presented.

CDC18L, CDC6

In yeasts, Cdc6 (Saccharomyces cerevisiae) and Cdc18 (Schizosaccharomyces pombe) associate with the origin recognition complex (ORC) proteins to render cells competent for DNA replication. Thus, Cdc6 has a critical regulatory role in the initiation of DNA replication in yeast. cDNAs encoding Xenopus and human homologues of yeast CDC6 have been isolated [Williams et al., 1997, (44)]. They designated the human and Xenopus proteins p62(cdc6). Independently, in a yeast 2-hybrid assay using PCNA as bait, cDNAs encoding the human CDC6/Cdc18 homologue have been isolated [Saha et al, 1998, (45)]. These authors reported that the predicted 560-amino acid human protein shares approximately 33% sequence identity with the 2 yeast proteins. On Western blots of HeLa cell extracts, human CDC6/cdc18 migrates as a 66-kD protein. Although Northern blots indicated that CDC6/Cdc 18 mRNA levels peak at the onset of S phase and diminish at the onset of mitosis in HeLa cells, the authors found that total CDC6/Cdc 18 protein level is unchanged throughout the cell cycle. Immunofluorescent analysis of epitope-tagged protein revealed that human CDC6/Cdc18 is nuclear in G1- and cytoplasmic in S-phase cells, suggesting that DNA replication may be regulated by either the translocation of this protein between the nucleus and cytoplasm or by selective degradation of the protein in the nucleus. Immunoprecipitation studies showed that human CDC6/Cdc18 associates in vivo with cyclin A, CDK2, and ORC1. The association of cyclin-CDK2 with CDC6/Cdc18 was specifically inhibited by a factor present in mitotic cell extracts. Therefore it has been suggested that if the interaction between CDC6/Cdc 18 with the S phase-promoting factor cyclin-CDK2 is essential for the initiation of DNA replication, the mitotic inhibitor of this interaction could prevent a premature interaction until the appropriate time in G1. Cdc6 is expressed selectively in proliferating but not quiescent mammalian cells, both in culture and within tissues in intact animals [Yan et al., 1998, (46)]. During the transition from a growth-arrested to a proliferative state, transcription of mammalian Cdc6 is regulated by E2F proteins, as revealed by a functional analysis of the human Cdc6 promoter and by the ability of exogenously expressed E2F proteins to stimulate the endogenous Cdc6 gene. Immunodepletion of Cdc6 by microinjection of anti-Cdc6 antibody blocked initiation of DNA replication in a human tumor cell line. The authors concluded that expression of human Cdc6 is regulated in response to mitogenic signals through transcriptional control mechanisms involving E2F proteins, and that Cdc6 is required for initiation of DNA replication in mammalian cells.

Using a yeast 2-hybrid system, co-purification of recombinant proteins, and immunoprecipitation, it has been demonstrated lateron that an N-terminal segment of CDC6 binds specifically to PR48, a regulatory subunit of protein phosphatase 2A (PP2A). The authors hypothesized that dephosphorylation of CDC6 by PP2A, mediated by a specific interaction with PR48 or a related B-double prime protein, is a regulatory event controlling initiation of DNA replication in mammalian cells. By analysis of somatic cell hybrids and by fluorescence in situ hybridization the human p62(cdc6) gene has been to 17q21.3.

### TOP2A, TOP2, TOP2 alpha

DNA topoisomerases are enzymes that control and alter the topologic states of DNA in both prokaryotes and eukaryotes. Topoisomerase II from eukaryotic cells catalyzes the relaxation of supercoiled DNA molecules, catenation, decatenation, knotting, and unknotting of circular DNA. It appears likely that the reaction catalyzed by topoisomerase II involves the crossing-over of 2 DNA segments. It has been estimated that there are about 100,000 molecules of topoisomerase II per HeLa cell nucleus, constituting about 0.1% of the nuclear extract. Since several of the abnormal characteristics of ataxia-telangiectasia appear to be due to defects in DNA processing, screening for these enzyme activities in 5 AT cell lines has been performed [Singh et al., 1988, (47)]. In comparison to controls, the level of DNA topoisomerase II, determined by unknotting of P4 phage DNA, was reduced substantially in 4 of these cell lines and to a lesser extent in the fifth. DNA topoisomerase I, assayed by relaxation of supercoil DNA, was found to be present at normal levels.

The entire coding sequence of the human TOP2 gene has been determined [Tsai-Pflugfelder et al., 1988, (48)].

In addition human cDNAs that had been isolated by screening a cDNA library derived from a mechlorethamine-resistant Burkitt lymphoma cell line (Raji-HN2) with a Drosophila Topo II cDNA had been sequenced [Chung et al., 1989, (49)]. The authors identified 2 classes of sequence representing 2 TOP2 isoenzymes, which have been named TOP2A and TOP2B. The sequence of 1 of the TOP2A cDNAs is identical to that of an internal fragment of the TOP2 cDNA isolated by Tsai-Pflugfelder et al., 1988 (48). Southern blot analysis indicated that the TOP2A and TOP2B cDNAs are derived from distinct genes. Northern blot analysis using a TOP2A-specific probe detected a 6.5-kb transcript in the human cell line U937. Antibodies against a TOP2A peptide recognized a 170-kD protein in U937 cell lysates. Therefore it was concluded that their data provide genetic and immunochemical evidence for 2 TOP2 isozymes. The complete structures of the TOP2A and TOP2B genes has been reported [Lang et al., 1998, (50)]. The TOP2A gene spans approximately 30 kb and contains 35 exons.

Tsai-Pflugfelder et al., 1988 (48) showed that the human enzyme is encoded by a single-copy gene which they mapped to 17q21-22 by a combination of in situ hybridization of a cloned fragment to metaphase chromosomes and by Southern hybridization analysis with a panel of mouse-human hybrid cell lines. The assignment to chromosome 17 has been confirmed by the study of somatic cell hybrids. Because of co-amplification in an adenocarcinoma cell line, it was concluded that the TOP2A and ERBB2 genes may be closely linked on chromosome 17 [Keith et al., 1992, (51)]. Using probes that detected RFLPs at both the TOP2A and TOP2B loci, the demonstrated heterozygosity at a frequency of 0.17 and 0.37 for the alpha and beta loci, respectively. The mouse homologue was mapped to chromosome 11 [Kingsmore et al., 1993, (52)]. The structure and function of type II DNA topoisomerases has been reviewed [Watt et al., 1994, (53)]. DNA topoisomerase II-alpha is associated with the pol II holoenzyme and is a required component of chromatin-dependent co-activation. Specific inhibitors of topoisomerase II blocked transcription on chromatin templates, but did not affect transcription on naked templates. Addition of purified topoisomerase II-alpha reconstituted chromatin-dependent activation activity in reactions with core pol II. Therefore the transcription on chromatin templates seems to result in the accumulation of superhelical tension, making the relaxation activity of topoisomerase II essential for productive RNA synthesis on nucleosomal DNA.

### IGFBP4

Six structurally distinct insulin-like growth factor binding proteins have been isolated and their cDNAs cloned: IGFBP1, IGFBP2, IGFBP3, IGFBP4, IGFBP5 and IGFBP6. The proteins display strong sequence homologies, suggesting that they are encoded by a closely related family of genes. The IGFBPs contain 3 structurally distinct domains each comprising approximately one-third of the molecule. The N-terminal domain 1 and the C-tenminal domain 3 of the 6 human IGFBPs show moderate to high levels of sequence identity including 12 and 6 invariant cysteine residues in domains 1 and 3, respectively (IGFBP6 contains 10 cysteine residues in domain 1), and are thought to be the IGF binding domains. Domain 2 is defined primarily by a lack of sequence identity among the 6 IGFBPs and by a lack of cysteine residues, though it does contain 2 cysteines in IGFBP4. Domain 3 is homologous to the thyroglobulin type I repeat unit. Recombinant human insulin-like growth factor binding proteins 4, 5, and 6 have been characterized by their expression in yeast as fusion proteins with ubiquitin [Kiefer et al., 1992, (54)]. Results of the study suggested to the authors that the primary effect of the 3 proteins is the attenuation of IGF activity and suggested that they contribute to the control of IGF-mediated cell growth and metabolism. Moreover, IGFBPs have influence on EGFR and Her-2/neu mediated signaling. Addition of IGFBPs to Her-2/neu overexpressing cells at least in part blocks growth and survival characteristics of the respective cells.

Based on peptide sequences of a purified insulin-like growth factor-binding protein (IGFBP) rat IGFBP4 has been cloned by using PCR [Shimasaki et al., 1990, (55)]. They used the rat cDNA to clone the human ortholog from a liver cDNA library. Human IGFBP4 encodes a 258-amino acid polypeptide, which includes a 21-amino acid signal sequence. The protein is very hydrophilic, which may facilitate its ability as a carrier protein for the IGFs in blood. Northern blot analysis of rat tissues revealed expression in all tissues examined, with highest expression in liver. It was stated that IGFBP4 acts as an inhibitor of IGF-induced bone cell proliferation. The genomic region containing the IGFBP gene. The gene consists of 4 exons spanning approximately 15 kb of genomic DNA has been examined [Zazzi et al., 1998, (56)]. The upstream region of the gene contains a TATA box and a cAMP-responsive promoter.

By in situ hybridization, the IGFBP4 gene was mapped to 17q12-q21 [Bajalica et al., 1992, (57)]. Because the hereditary breast-ovarian cancer gene BRCA1 had been mapped to the same region, it has been investigated whether IGFBP4 is a candidate gene by linkage analysis of 22 BRCA1 families; the finding of genetic recombination suggested that it is not the BRCA1 gene [Tonin et al., 1993, (58)].

### EBI 1, CCR7, CMKBR7

Using PCR with degenerate oligonucleotides, a lymphoid-specific member of the G protein-coupled receptor family has been identified and mapped mapped to 17q12-q21.2 by analysis of human/mouse somatic cell hybrid DNAs and fluorescence in situ hybridization. It has been shown that this receptor had been independently identified as the Epstein-Barr-induced cDNA (symbol EBI1) [Birkenbach et al., 1993, (59)]. EBI1 is expressed in normal lymphoid tissues and in several B- and T-lymphocyte cell lines. While the function and the ligand for EBI1 remains unknown, its sequence and gene structure suggest that it is related to receptors that recognize chemoattractants, such as interleukin-8, RANTES, C5a, and fMet-Leu-Phe. Like the chemoattractant receptors, EBI1 contains intervening sequences near its 5-prime end; however, EBI1 is unique in that both of its introns interrupt the coding region of the first extracellular domain. Mouse Ebi1 cDNA has been isolated and found to encode a protein with 86% identity to the human homologue.

Subsets of murine CD4+ T cells localize to different areas of the spleen after adoptive transfer. Naive and T helper-1 (TH1) cells, which express CCR7, home to the periarteriolar lymphoid sheath, whereas activated TH2 cells, which lack CCR7, form rings at the periphery of the T-cell zones near B-cell follicles. It has been found that retroviral transduction of TH2 cells with CCR7 forced them to localize in a TH1-like pattern and inhibited their participation in B-cell help in vivo but not in vitro. Apparently differential expression of chemokine receptors results in unique cellular migration patterns that are important for effective immune responses.

CCR7 expression divides human memory T cells into 2 functionally distinct subsets. CCR7-memory cells express receptors for migration to inflamed tissues and display immediate effector function. In contrast, CCR7⁺ memory cells express lymph node homing receptors and lack immediate effector function, but efficiently stimulate dendritic cells and differentiate into CCR7⁻ effector cells upon secondary stimulation. The CCR7⁺ and CCR7⁻ T cells, named central memory (T-CM) and effector memory (T-EM), differentiate in a step-wise fashion from naive T cells, persist for years after immunization, and allow a division of labor in the memory response.

CCR7 expression in memory CD8⁺ T lymphocyte responses to HIV and to cytomegalovirus (CMV) tetramers has been evaluated. Most memory T lymphocytes express CD45RO, but a fraction express instead the CD45RA marker. Flow cytometric analyses of marker expression and cell division identified 4 subsets of HIV- and CMV-specific CD8⁺ T cells, representing a lineage differentiation pattern: CD45RA⁺CCR7⁺ (double-positive); CD45RA⁻CCR7⁺; CD45RA⁻CCR7⁻ (double-negative); CD45RA⁺CCR7⁻. The capacity for cell division, as measured by 5-(and 6-)carboxyl-fluorescein diacetate, succinimidyl ester, and intracellular staining for the Ki67 nuclear antigen, is largely confined to the CCR7⁺ subsets and occurred more rapidly in cells that are also CD45RA⁺. Although the double-negative cells did not divide or expand after stimulation, they did revert to positivity for either CD45RA or CCR7 or both. The CD45RA⁺CCR7⁻ cells, considered to be terminally differentiated, fail to divide, but do produce interferon-gamma and express high levels of perforin. The representation of subsets specific for CMV and for HIV is distinct. Approximately 70% of HIV-specific CD8⁺ memory T cells are double-negative or preterminally differentiated compared to 40% of CMV-specific cells. Approximately 50% of the CMV-specific CD8+ memory T cells are terminally differentiated compared to fewer than 10% of the HIV-specific cells. It has been proposed that terminally differentiated CMV-specific cells are poised to rapidly intervene, while double-positive precursor cells remain for expansion and replenishment of the effector cell pool. Furthermore, high-dose antigen tolerance and the depletion of HIV-specific CD4⁺ helper T-cell activity may keep the HIV-specific memory CD8⁺ T cells at the double-negative stage, unable to differentiate to the terminal effector state. B lymphocytes recirculate between B cell-rich compartments (follicles or B zones) in secondary lymphoid organs, surveying for antigen. After antigen binding, B cells move to the boundary of B and T zones to interact with T-helper cells. Furthermore it has been demonstrated that antigen-engaged B cells have increased expression of CCR7, the receptor for the T-zone chemokines CCL19 (also known as ELC) and CCL21, and that they exhibit increased responsiveness to both chemoattractants. In mice lacking lymphoid CCL19 and CCL21 chemokines, or with B cells that lack CCR7, antigen engagement fails to cause movement to the T zone. Using retroviral-mediated gene transfer, the authors demonstrated that increased expression of CCR7 is sufficient to direct B cells to the T zone. Reciprocally, overexpression of CXCR5, the receptor for the B-zone chemokine CXCL13, is sufficient to overcome antigen-induced B-cell movement to the T zone. This points toward a mechanism of B-cell relocalization in response to antigen, and established that cell position in vivo can be determined by the balance of responsiveness to chemoattractants made in separate but adjacent zones.

### BAF57, SMARCE 1

The SWI/SNF complex in S. cerevisiae and Drosophila is thought to facilitate transcriptional activation of specific genes by antagonizing chromatin-mediated transcriptional repression. The complex contains an ATP-dependent nucleosome disruption activity that can lead to enhanced binding of transcription factors. The BRG1/brm-associated factors, or BAF, complex in mammals is functionally related to SWI/SNF and consists of 9 to 12 subunits, some of which are homologous to SWI/SNF subunits. A 57-kD BAF subunit, BAF57, is present in higher eukaryotes, but not in yeast. Partial coding sequence has been obtained from purified BAF57 from extracts of a human cell line [Wang et al., 1998, (60)]. Based on the peptide sequences, they identified cDNAs encoding BAF57. The predicted 411-amino acid protein contains an HMG domain adjacent to a kinesin-like region. Both recombinant BAF57 and the whole BAF complex bind 4-way junction (4WJ) DNA, which is thought to mimic the topology of DNA as it enters or exits the nucleosome. The BAF57 DNA-binding activity has characteristics similar to those of other HMG proteins. It was found that complexes with mutations in the BAF57 HMG domain retain their DNA-binding and nucleosome-disruption activities. They suggested that the mechanism by which mammalian SWI/SNF-like complexes interact with chromatin may involve recognition of higher-order chromatin structure by 2 or more DNA-binding domains. RNase protection studies and Western blot analysis revealed that BAF57 is expressed ubiquitously. Several lines of evidence point toward the involvement of SWI/SNF factors in cancer development [Klochendler-Yeivin et al., 2002, (61)]. Moreover, SWI/SNF related genes are assigned to chromosomal regions that are frequently involved in somatic rearrangements in human cancers [Ring et al., 1998, (62)]. In this respect it is interesting that some of the SWI/SNF family members (i.e. SMARCC1, SMARCC2, SMARCD1 and SMARCD22 are neighboring 3 of the eucaryotic ARCHEONs we have identified (i.e. 3p21-p24, 12q13-q14 and 17q respectively) and which are part of the present invention. In this invention we could also map SMARCE1/BAF57 to the 17q12 region by PCR karyotyping.

### KRT 10, K10

Keratin 10 is an intermediate filament (IF) chain which belongs to the acidic type I family and is expressed in terminally differentiated epidermal cells. Epithelial cells almost always co-express pairs of type I and type I keratins, and the pairs that are co-expressed are highly characteristic of a given epithelial tissue. For example, in human epidermis, 3 different pairs of keratins are expressed: keratins 5 (type II) and 14 (type I), characteristic of basal or proliferative cells; keratins 1 (type II) and 10 (type I), characteristic of superbasal terminally differentiating cells; and keratins 6 (type II) and 16 (type I) (and keratin 17 [type I]), characteristic of cells induced to hyper-proliferate by disease or injury, and epithelial cells grown in cell culture. The nucleotide sequence of a 1,700 bp cDNA encoding human epidermal keratin 10 (56.5 kD) [Darmon et al., 1987, (63)] has been published as well as the complete amino acid sequence of human keratin 10 [Zhou et al., 1988, (64)]. Polymorphism of the KRT10 gene, restricted to insertions and deletions of the glycine-richquasipeptide repeats that form the glycine-loop motif in the C-terminal domain, have been extensively described [Korge et al., 1992, (65)].

By use of specific cDNA clones in conjunction with somatic cell hybrid analysis and in situ hybridization, KRT10 gene has been mapped to 17q12-q21 in a region proximal to the breakpoint at 17q21 that is involved in a t(17;21)(q21;q22) translocation associated with a form of acute leukemia. KRT10 appeared to be telomeric to 3 other loci that map in the same region: CSF3, ERBA1, and HER2 [Lessin et al., 1988, (66)]. NGFR and HOX2 are distal to K9. It has been demonstrated that the KRT10, KRT13, and KRT15 genes are located in the same large pulsed field gel electrophoresis fragment [Romano et al., 1991, (67)]. A correlation of assignments of the 3 genes makes 17q21-q22 the likely location of the cluster. Transgenic mice expressing a mutant keratin 10 gene have the phenotype of epidermolytic hyperkeratosis, thus suggesting that a genetic basis for the human disorder resides in mutations in genes encoding suprabasal keratins KRT1 or KRT10 [Fuchs et al 1992, (68)]. The authors also showed that stimulation of basal cell proliferation can result from a defect in suprabasal cells and that distortion of nuclear shape or alterations in cytokinesis can occur when an intermediate filament network is perturbed. In a family with keratosis palmaris et plantaris without blistering either spontaneously or in response to mild mechanical or thermal stress and with no involvement of the skin and parts of the body other than the palms and soles, a tight linkage to an insertion-deletion polymorphism in the C-terminal coding region of the KRT10 gene (maximum lod score=8.36 at theta=0.00) was found [Rogaev et al., 1993, (69)]. It is noteworthy that it was a rare, high molecular weight allele of the KRT10 polymorphism that segregated with the disorder. The allele was observed once in 96 independent chromosomes from unaffected Caucasians. The KRT10 polymorphism arose from the insertion/deletion of imperfect (CCG)n repeats within the coding region and gave rise to a variable glycine loop motif in the C-terminal tail of the keratin 10 protein. It is possible that there was a pathogenic role for the expansion of the imperfect trinucleotide repeat.

### KRT12, K12

Keratins are a group of water-insoluble proteins that form 10 nm intermediate filaments in epithelial cells. Approximately 30 different keratin molecules have been identified. They can be divided into acidic and basic-neutral subfamilies according to their relative charges, immunoreactivity, and sequence homologies to types I and II wool keratins, respectively. In vivo, a basic keratin usually is co-expressed and ‘paired’ with a particular acidic keratin to form a heterodimer. The expression of various keratin pairs is tissue specific, differentiation dependent, and developmentally regulated. The presence of specific keratin pairs is essential for the maintenance of the integrity of epithelium. For example, mutations in human K14/K5 pair and the K10/K1 pair underlie the skin diseases, epidermolysis bullosa simplex and epidermolytic hyperkeratosis, respectively. Expression of the K3 and K12 keratin pair have been found in the cornea of a wide number of species, including human, mouse, and chicken, and is regarded as a marker for corneal-type epithelial differentiation. The murine Krt12 (Krt1.12) gene and demonstrated that its expression is corneal epithelial cell specific, differentiation dependent, and developmentally regulated [Liu et al., 1993, (70)]. The corneal-specific nature of keratin 12 gene expression signifies keratin 12 plays a unique role in maintaining normal corneal epithelial function. Nevertheless, the exact function of keratin 12 remains unknown and no hereditary human corneal epithelial disorder has been linked directly to the mutation in the keratin 12 gene. As part of a study of the expression profile of human corneal epithelial cells, a cDNA with an open reading frame highly homologous to the cornea-specific mouse keratin 12 gene has been isolated [Nishida et al., 1996, (71)]. To elucidate the function of keratin 12 knockout mice lacking the Krt1.12 gene have been created by gene targeting techniques. The heterozygous mice appeared normal. Homozygous mice developed normally and suffered mild corneal epithelial erosion. The corneal epithelia were fragile and could be removed by gentle rubbing of the eyes or brushing. The corneal epithelium of the homozygotes did not express keratin 12 as judged by immunohistochemistry, Western immunoblot analysis with epitope-specific anti-keratin 12 antibodies, Northern hybridization, and in situ hybridization with an antisense keratin 12 riboprobe. The KRT12 gene has been mapped to 17q by study of radiation hybrids and localized it to the type I keratin cluster in the interval between D17S800 and D17S930 (17q12-21) [Nishida et al., 1997, (72)]. The authors presented the exon-intron boundary structure of the KRT12 gene and mapped the gene to 17q12 by fluorescence in situ hybridization. The gene contains 7 introns, defining 8 exons that cover the coding sequence. Together the exons and introns span approximately 6 kb of genomic DNA.

Meesmann corneal dystrophy is an autosomal dominant disorder causing fragility of the anterior corneal epithelium, where the cornea-specific keratins K3 and K12 are expressed. Dominant-negative mutations in these keratins might be the cause of Meesmann corneal dystrophy. Indeed, linkage of the disorder to the K12 locus in Meesmann's original German kindred [Meesmann and Wilke, 1939, (73)] with Z(max)=7.53 at theta=0.0 has been found. In 2 pedigrees from Northern Ireland, they found that the disorder co-segregated with K12 in one pedigree and K3 in the other. Heterozygous missense mutations in K3 or in K12 (R135T, V143L,) in each family have been identified. All these mutations occurred in highly conserved keratin helix boundary motifs, where dominant mutations in other keratins have been found to compromise cytoskeletal function severely, leading to keratinocyte fragility.

The regions of the human KRT12 gene have been sequenced to enable mutation detection for all exons using genomic DNA as a template [Corden et al., 2000, (74)]. The authors found that the human genomic sequence spans 5,919 bp and consists of 8 exons. A microsatellite dinucleotide repeat was identified within intron 3, which was highly polymorphic and which they developed for use in genotype analysis. In addition, 2 mutations in the helix initiation motif of K12 were found in families with Meesmann corneal dystrophy. In an American kindred, a missense M129T mutation was found in the KRT12 gene. They stated that a total of 8 mutations in the KRT12 gene had been reported.

### Genetic Interactions within ARCHEONs

Genes involved in genomic alterations (amplifications, insertions, translocations, deletions, etc.) exhibit changes in their expression pattern. Of particular interest are gene amplifications, which account for gene copy numbers >2 per cell or deletions accounting for gene copy numbers <2 per cell. Gene copy number and gene expression of the respective genes do not necessarily correlate. Transcriptional overexpression needs an intact transcriptional context, as determined by regulatory regions at the chromosomal locus (promotor, enhancer and silencer), and sufficient amounts of transcriptional regulators being present in effective combinations. This is especially true for genomic regions, which expression is tightly regulated in specific tissues or during specific developmental stages. ARCHEONs are specified by gene clusters of more than two genes being directly neighbored or in chromosomal order, interspersed by a maximum of 10, preferably 7, more preferably 5 or at least 1 gene. The interspersed genes are also co-amplified but do not directly interact with the ARCHEON. Such an ARCHEON may spread over a chromosomal region of a maximum of 20, more preferably 10 or at least 6 Megabases. The nature of an ARCHEON is characterized by the simultaneous amplification and/or deletion and the correlating expression (i.e. upregulation or downregulation respectively) of the encompassed genes in a specific tissue, cell type, cellular or developmental state or time point. Such ARCHEONs are commonly conserved during evolution, as they play critical roles during cellular development. In case of these ARCHEONs whole gene clusters are overexpressed upon amplification as they harbor self-regulatory feedback loops, which stabilize gene expression and/or biological effector function even in abnormal biological settings, or are regulated by very similar transcription factor combinations, reflecting their simultaneous function in specific tissues at certain developmental stages. Therefore, the gene copy numbers correlates with the expression level especially for genes in gene clusters functioning as ARCHEONs. In case of abnormal gene expressions in neoplastic lesions it is of great importance to know whether the self-regulatory feedback loops have been conserved as they determine the biological activity of the ARCHEON gene members.

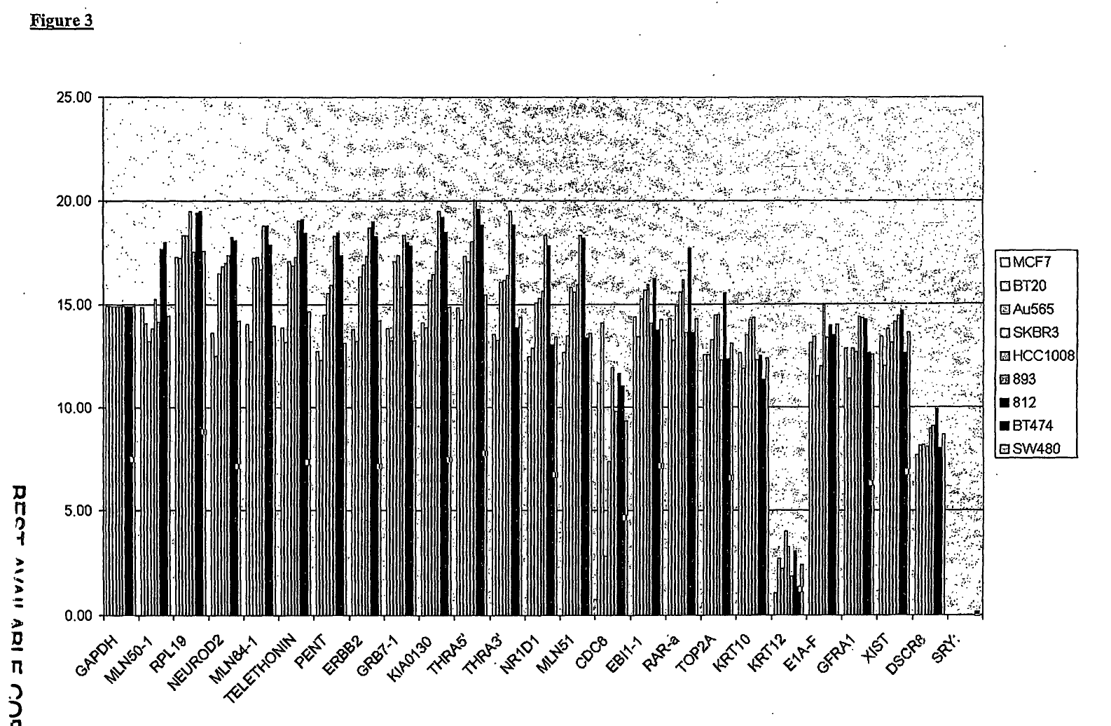

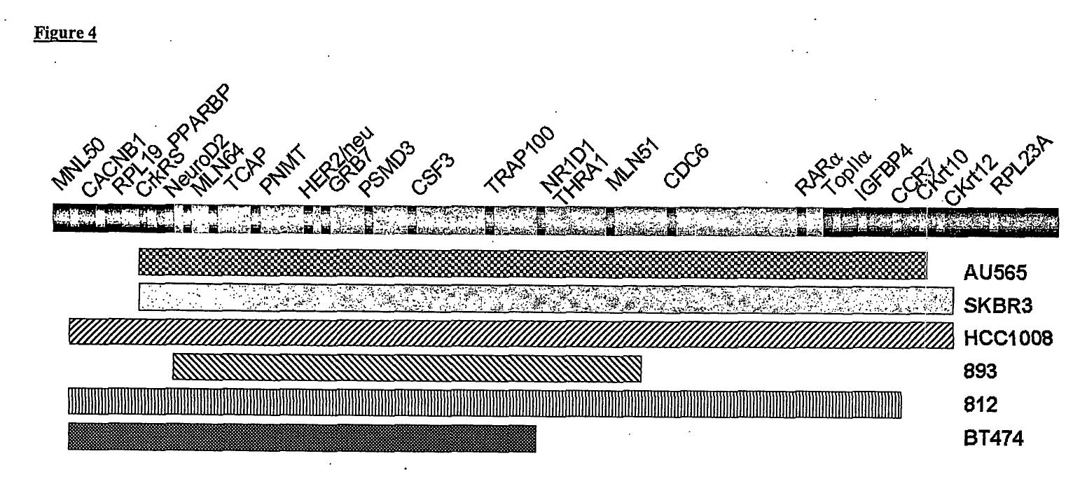

The intensive interaction between genes in ARCHEONs is described for the 17q21 ARCHEON (FIG. 1) by way of illustration not by limitation. In one embodiment the presence or absence of alterations of genes within distinct genomic regions are correlated with each other, as exemplified for breast cancer cell lines (FIG. 3 and FIG. 4). This confers to the discovery of the present invention, that multiple interactions of said gene products of defined chromosomal localizations happen, that according to their respective alterations in abnormal tissue have predictive, diagnostic, prognostic and/or preventive and therapeutic value. These interactions are mediated directly or indirectly, due to the fact that the respective genes are part of interconnected or independent signaling networks or regulate cellular behavior (differentiation status, proliferative and/or apoptotic capacity, invasiveness, drug responsiveness, immune modulatory activities) in a synergistic, antagonistic or independent fashion. The order of functionally important genes within the ARCHEONs has been conserved during evolution (e.g. the ARCHEON on human chromosome 17q21 is present on mouse chromosome 11). Moreover, it has been found that the 17q21 ARCHEON is also present on human chromosome 3p21 and 12q13, both of which are also involved in amplification events and in tumor development. Most probably these homologous ARCHEONs were formed by duplications and rearrangements during vertebrate evolution. Homologous ARCHEONs consist of homologous genes and/or isoforms of specific gene families (e.g. RARA or RARB or RARG, THRA or THRB, TOP2A or TOP2B, RAB5A or RAB5B, BAF170 or BAF 155, BAF60A or BAF60B, WNT5A or WNT5B, IGFBP4 or IGFBP6). Moreover these regions are flanked by homologous chromosomal gene clusters (e.g. CACN, SCYA, HOX, Keratins). These ARCHEONs have diverged during evolution to fulfill their respective functions in distinct tissues (e.g. the 17q21 ARCHEON has one of its main functions in the central nervous system). Due to their tissue specific function extensive regulatory loops control the expression of the members of each ARCHEON. During tumor development these regulations become critical for the characteristics of the abnormal tissues with respect to differentiation, proliferation, drug responsiveness, invasiveness. It has been found that the co-amplification of genes within ARCHEONs can lead to co-expression of the respective gene products. Some of said genes also exhibit additional mutations or specific patterns of polymorphisms, which are substantial for the oncogenic capacities of these ARCHEONs. It is one of the critical features of such amplicons, which members of the ARCHEON have been conserved during tumor formation (e.g. during amplification and deletion events), thereby defining these genes as diagnostic marker genes. Moreover, the expression of the certain genes within the ARCHEON can be influenced by other members of the ARCHEON, thereby defining the regulatory and regulated genes as target genes for therapeutic intervention. It was also observed, that the expression of certain members of the ARCHEON is sensitive to drug treatment (e.g. TOPO2 alpha, RARA, THRA, HER-2) which defines these genes as “marker genes”. Moreover several other genes are suitable for therapeutic intervention by antibodies (CACNB1, EBI1), ligands (CACNB1) or drugs like e.g. kinase inhibitors (CrkRS, CDC6). The following examples of interactions between members of ARCHEONs are offered by way of illustration, not by way of limitation.

EBI1/CCR7 is lymphoid-specific member of the G protein-coupled receptor family. EBI1 recognizes chemoattractants, such as interleukin-8, SCYAS, Rantes, CSa, and fMet-Leu-Phe. The capacity for cell division is largely confined to the CCR7⁺ subsets in lymphocytes. Double-negative cells did not divide or expand after stimulation. CCR7⁻ cells, considered to be terminally differentiated, fail to divide, but do produce interferon-gamma and express high levels of perforin. EBI1 is induced by viral activities such as the Eppstein-Barr-Virus. Therefore, EBI1 is associated with transformation events in lymphocytes. A functional role of EBI1 during tumor formation in non-lymphoid tissues has been investigated in this invention. Interestingly, also ERBA and ERBB, located in the same genomic region, are associated with lymphocyte transformation. Moreover, ligands of the receptor (i.e. SCYA5/Rantes) are in genomic proximity on 17q. Abnormal expression of both of these factors in lymphoid and non-lymphoid tissues establishes an autorgulatory feedback loop, inducing signaling events within the respective cells. Expression of lymphoid factors has effect on immune cells and modulates cellular behavior. This is of particular interest with regard to abnormal breast tissue being infiltrated by lymphocytes. In line with this, another immunmodulatory and proliferation factor is located nearby on 17q21. Granulocyte colony-stimulating factor (GCSF3) specifically stimulates the proliferation and differentiation of the progenitor cells for granulocytes. A stimulatory activity from a glioblastoma multiforme cell line being biologically and biochemically indistinguishable from GCSF produced by a bladder cell line has also been found. Colony-stimulating factors not only affects immune cells, but also induce cellular responses of non-immune cells, indicating possible involvement in tumor development upon abnormal expression. In addition several other genes of the 17q21 ARCHEON are involved in proliferation, survival, differentiation of immune cells and/or lymphoblastic leukemia, such as MLLT6, ZNF144 and ZNFN1A3, again demonstrating the related functions of the gene products in interconnected key processes within specific cell types. Aberrant expression of more than one of these genes in non-immune cells constitutes signalling activities, that contribute to the oncogenic activities that derive solely from overexpression of the Her-2/neu gene.

PPARBP has been found in complex with the tumorsuppressor gene of the p53 family. Moreover, PPARBP also binds to PPAR-alpha (PPARA), RAR-alpha (RARA), RXR, THRA and TR-beta-1. Due to it's ability to bind to thyroid hormone receptors it has been named TRIP2 and TRAP220. In this complexes PPARBP affects gene regulatory activities. Interestingly, PPARBP is located in genomic proximity to its interaction partners THRA and RARA. We have found PPARBP to be co-amplified with THRA and RARA in tumor tissue. THRA has been isolated from avian erytbroblastosis virus in conjunction with ERBB and therefore was named ERBA. ERBA potentiates ERBB by blocking differentiation of erythroblasts at an immature stage. ERBA has been shown to influence ERBB expression. In this setting deletions of C-terminal portions of the THRA gene product are of influence. Aberrant THRA expression has also been found in nonfunctioning pituitary tumors, which has been hypothesized to reflect mutations in the receptor coding and regulatory sequences. THRA function promotes tumor cell development by regulating gene expression of regulatory genes and by influencing metabolic activities (e.g. of key enzymes of alternative metabolic pathways in tumors such as malic enzyme and genes responsible for lipogenesis). The observed activities of nuclear receptors not only reflect their transactivating potential, but are also due to posttranscriptional activities in the absence or presence of ligands. Co-amplification of THRA/ERBA and ERBB has been shown, but its influence on tumor development has been doubted as no overexpression could be demonstrated in breast tumors [van de Vijver et al., 1987, (75)]. THRA and RARA are part of nuclear receptor family whose function can be mediated as monomers, homodimers or heterodimers. RARA regulates differentiation of a broad spectrum of cells. Interactions of hormones with ERBB expression has been investigated. Ligands of RARA can inhibit the expression of amplified ERBB genes in breast tumors [Offterdinger et al., 1998, (76)]. As being part of this invention co-amplification and co-expression of ThRA and RARA could be shown. It was also found that multiple genes, which are regulated by members of the thyroid hormone receptor—and retinoic acid receptor family, are differentially expressed in tumor samples, corresponding to their genomic alterations (amplification, mutation, deletion). These hormone receptor genes and respective target genes are useful to discriminate patient samples with respect to clinical features.

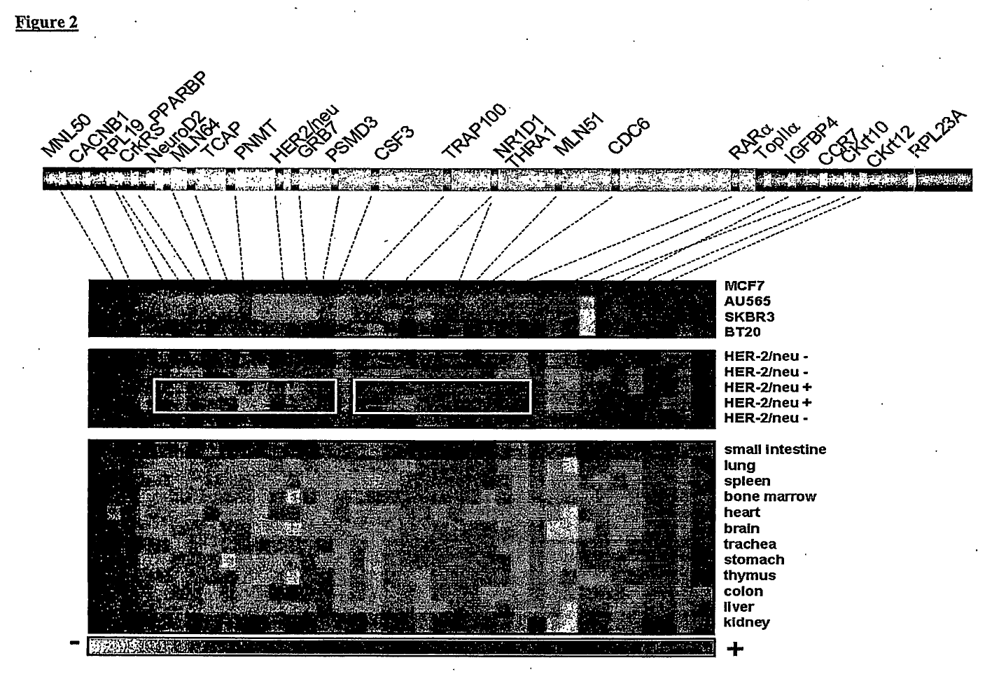

By expression analysis of multiple normal tissues, tumor samples and tumor cell lines and subsequent clustering of the 17q21 region, it was found that the expression profile of Her-2/neu positive tumor cells and tumor samples exhibits similarities with the expression pattern of tissue from the central nervous system (FIG. 2). This is in line with the observed malformations in the central nervous system of Her-2/neu and THRA knock-out mice. Moreover, it was found that NEUROD2, a nuclear factor involved specifically in neurogenesis, is commonly expressed in the respective samples. This led to the definition of the 17q21 Locus as being an “ARCHEON”, whose primary function in normal organ development is defined to the central nervous system. Surprisingly, the expression of NEUROD2 was affected by therapeutic intervention. Strikingly, also ZNF144, TEM7, PIP5K and PPP1R1B are expressed in neuronal cells, where they display diverse tissue specific functions.

In addition Her-2/neu is often co-amplified with GRB7, a downstream member of the signaling cascade being involved in invasive properties of tumors. Surprisingly, we have found another member of the Her-2/neu signaling cascade being overexpressed in primary breast tumors TOB1 (=“Transducer of ERBB signaling”). Strong overexpression of TOB1 corellated with weaker overexpression of Her-2/neu, already indicating its involvement in oncogenic signaling activities. Amplification of Her-2/neu has been assigned to enhanced proliferative capacity, due to the identified downstream components of the signaling cascade (e.g. Ras-Raf-MAPK). In this respect it was surprising that some cdc genes, which are cell cycle dependent kinases, are part of the amplicons, which upon altered expression have great impact on cell cycle progression.

The ARCHEONS on 17q21 and 12q13 are very closely related, as they do not only harbor isoforms of specific genes (e.g. CACNB1 vs. CACNB3, ERBB2 vs. ERBB3, RARA vs. RARG, see below), but are even flanked by whole gene clusters, consisting of multiple isoforms of one gene family positioned in tandem, such as the keratin and the HOX gene cluster. In this respect the simultaneous presence of keratins and receptors of the EGFR family, i.e. ERBB2 (Her-2/neu) and ERBB3 (Her-3) is of special interest, as the expression of individual keratins is very tightly controlled by the EGFR signalling pathway.

Keratins are a group of water-insoluble proteins that form 10 nm intermediate filaments in epithelial cells. Approximately 30 different keratin molecules have been identified. They can be divided into acidic and basic-neutral subfamilies according to their relative charges, immunoreactivity, and sequence homologies to types I and II wool keratins, respectively. In vivo, a basic keratin usually is co-expressed and ‘paired’ with a particular acidic keratin to form a heterodimer. The expression of various keratin pairs is tissue specific, differentiation dependent, and developmentally regulated. The presence of specific keratin pairs is essential for the maintenance of the integrity of epithelium. Alterations of keratin expression have been observed in tumor epithelium, with an abnormal keratin pattern being expressed in tumor cells compared to the adjacent normal tissue. Mutations in human K14/K5 pair and the K10/K1 pair underlie skin diseases such as epidermolysis bullosa simplex and epidermolytic hyperkeratosis. The expression of these and other keratins within the skin is tightly regulated. For example, the expression of K14/K5 pair is restricted to the basal cell layer of the skin displaying no overlap with the K10/K1 pair, which is solely expressed in the suprabasal layer. Gene expression is very tightly controlled by an interplay of multiple signalling cascades such as the EGFR, TGFR, sonic hedgehog and wnt-signaling, involving receptor tyrosine kinases and serin threonin kinases. In addition, posttranslational modifications such as serine/threonine and/or tyrosine phosphorylation events affect keratin function, and can be attributed to receptor tyrosine kinase signalling and MAPK and ERK activity. Posttranlational modifications of keratins not only alters the solubility of keratins, but also affects nuclear and signalling functions (e.g. after association with 14-3-3 protein). In addition, we did observe genomic alteration of the keratin gene clusters perturbing keratin expression pattern.

Moreover, the physical interaction of keratins, which are located in ARCHEONs of different chromosomes and whose cell type specific expression at distinct differentiation status is regulated by members of the same ARCHEONs is a superb example of the genetic interaction of ARCHEON genes. Examples of this tight interaction between the 12q13 and 17q21 ARCHEONs are the expression and physical interaction of keratin 5 (basic keratin Type II located on 12q13) and keratin 14 (acidic keratin Type I located on 17q21) in the basal layer of the skin, which is shut off in the suprabasal layer and compensated by the expression and physical interaction of keratin 1 (basic keratin Type II located on 12q13) and keratin 10 (acidic keratin Type I located on 17q21). Diverse control mechanisms confer this exclusive expression control including chromosomal positioning and growth factor signaling activities. Interestingly, critical keratins are chromosomally positioned in an ordered fashion reflecting their related but exclusive function in different keratin pairs and in specific tissues, resembling the structure and function relationship of the hox gene clusters on the same chromosomes. Moreover, keratins whose mutation result in specific skin disorders (e.g. mutation of K5 and K14 results in hand and foot syndrom) are located at similar positions within the ARCHEONs on chromosome 17q21 and 12q13. The genes are in close proximity to genes involved in signaling events (e.g. ERBBs and RARs) regulating proliferation, differentiation and apoptotic events also in the skin tissue. For example Her-2/neu is specifically expressed within the basal layer of the skin, where asymmetric cell divisions of adult stem cells or early progenitor cells thereof give rise to a non-differentiated daughter cell residing in the basal layer and a differentiating daughter cell which is subsequently moving to the suprabasal compartment. These assymetric cell divisions guarantee the self-renewal and the cellular homeostasis of the skin tissue. This is of importance for the biological functions of the skin such as barrier function towards the environmental stress including infectious agents. Perturbation of the signalling activities within the skin results in diseases similar to the hereditary disorders reflecting mutations of specific keratin genes. In clinical studies it has been shown, that blocking EGFR signalling by antibody-treatment (e.g. cetuximab) and small molecule inhibitors (e.g. Iressa) targeted to the receptor tyrosin kinases can result in skin diseases (e.g. acne-like rash) of grade I, II or III. It is part of this invention, that these skin diseases not only reflect side effects of the respective treatments, but are an example for systemic changes occurring as a consequence of therapeutic regimen, thereby indicating suscebility of the endogenous signaling network to the therapeutic agents. This observation can have consequences on therapeutic decisions, as the therapeutic regimen are normally stopped or is reduced upon occurrence of side effects. However, as the side effects (e.g. the skin diseases occurring under anti growth factor treatment) are indicative of response to treatment (e.g. tumor shrinkage), the treatment should be endured even though “adverse” drug responses occur and side effects should be treated separately by agents softening the symptoms. Skin diseases such as rash and hand and foot syndrome are just examples for a given side effect under a given treatment (i.e. anti tumor therapy), that can be used for response correlation.

Similarly to blocking receptor molecules itself, blocking downstream members of these signaling cascades results mainly in skin diseases (e.g. hand-and-foot syndroms). Surprisingly, we did observe, that treating tumor cells with agents blocking the EGFR/Her-2/neu signaling (e.g. Cetuximab, Iressa, Herceptin, RAF kinase inhibitor, etc.) shifts the expression of specific keratins being part of the ARCHEONS described in this invention. Moreover, the altered expression of keratins in tumor cells of patients is paralleled by a shift of keratin expression in the keratinocytes of the skin of the very same patient. Perturbation of keratin expression and or post-transcriptional modification in the skin tissue seems to resemble the suscebility of the endogenous growth factor signaling pathways to the respective treatment. The resulting skin diseases are therefore at least to some extent indicative of the tumor responsiveness to the regimen. This endogenous responsiveness to anti growth factor signaling agents can also be delineated from polymorphisms and genetic alterations (e.g. mutations) being present within the ARCHEON described in this invention. Of particular interest are in this context polymorphisms being present in the keratin genes. However, polymorphisms within keratins, keratin related genes and/or genes functionally connected to the keratin-based cytoskeleton, not necessarily being present within the ARCHEONs described, are also of importance according to their physical interaction with the respective gene products (e.g. ITGB4). It is part of this invention, that the responsiveness of a given tumor to anti growth factor treatment relates to the genetic predisposition of the respective signaling pathway members and target genes, which include keratins and related genes, that are markers for proliferation, differentiation and apoptosis in normal tissues, such as skin tissue. This knowledge can be used to predict the responsiveness of a tumor based on the characterization of surrogate tissues, such as skin, blood and any other normal tissue containing the above mentioned genes and/or gene products. For example the responsiveness to Iressa, RAF-kinase inhibitor and antibody based therapies targeting EGFR and Her-2/neu can be delineated from punch biopsies of the skin (preferably by comparison of pre- and/or post-treatment samples) or blood samples by determining the expression pattern or genetic characterization of keratin or keratin-related genes of an individual patient. Moreover, the responsiveness of such surrogate tissues can then be correlated to the tumor phenotype and the responsiveness of a tumor to the respective treatment, thereby predict therapy outcome. The examples of surrogate tissues are given by way of illustration and not by limitation.

It is yet another embodiment of the invention, that adverse drug responses such as heart toxicities can also be deduced from characteristics of the ARCHEONs described. Of particular interest are the ARCHEONs at 17q12-24, 12q13 and 3q21-26. It is known that anthracyclin based, anti-cancer regimens result in heart toxicities (such as dilated cardiomyopathies), as can be deduced e.g. by LVEF measurements. Moreover, anthracyclin pretreated patients have significantly increased heart toxicity events upon subsequent Herceptin™ based regimen. Interestingly, the ARCHEONs described in this invention not only harbor the primary targets of these therapies (i.e. topoisomerases and Her-2/neu), but also important structural and functional genes (Telethonin, PNMT, CACNB1, PPARBP, Her-2/neu, Her4) for muscle function including heart muscle function. These genes are involved in central processes of heart muscle function, such as tyrosine phosphorylation, serine/threonine phosphorylation, calcium influx, regulating e.g. central structural proteins such as titin. Moreover, these genes can be colocalized in heart muscles, displaying their functional interplay in this tissue. In mouse models, the mislocalization of telethonin and the genetic inactivation of Her-2/neu, Her4 and Neuregulin result in a similar phenotype as can be seen for cancer patients being treated with diverse anti-cancer drugs. The synergistic adverse drug response effect seen for the combinatorial treatment with anthracyclin and Herceptin™. Delineation of polymorphisms and haplotypes of the respective genes, genomic region and/or the ARCHEON structure are indicative of the susceptibility to suffer from heart toxicities upon anti-cancer drug treatment. This is important for therapy decisions and cancer treatment management, as the prior therapies conducted exclude subsequent treatment options. For example, anthracyclin-based pretreatment can exclude subsequent Herceptin™ treatment or lead to reduced dosages, if possible heart toxicities (e.g. dilated cardiomyopathies) cannot be excluded.

According to the observations described above the following examples of genes at 3q21-26 are offered by way of illustration, not by way of limitation.
WNT5A, CACNA1D, THRB, RARB, TOP2B, RAB5B, SMARCC1 (BAF155), RAF, WNT7A

The following examples of genes at 12q13 are offered by way of illustration, not by way of limitation.
CACNB3, Keratins, ERBB3, NR4A1, RAB5/13, RARG, STAT6, WNT10B, (GCN5), (SAS: Sarcoma Amplified Sequence), SMARCC2 (BAF170), SMARCD1 (BAF60A), (GAS41: Glioma Amplified Sequence), (CHOP), Her3, KRTHB, HOX C, IGFBP6, WNT5B

There is cross-talk between the amplified ARCHEONs described above and some other highly amplified genomic regions locate approximately at 1p13, 1q32, 2p16, 2q21, 3p12, 5p13, 6p12, 7p12, 7q21, 8q23, 11q13, 13q12, 19q13, 20q13 and 21q11. The above mentioned chromosomal regions are described by way of illustration not by way of limitation, as the amplified regions often span larger and/or overlapping positions at these chromosomal positions.

Additional alterations of non-transcribed genes, pseudogenes or intergenic regions of said chromosomal locations can be measured for prediction, diagnosis, prognosis, prevention and treatment of malignant neoplasia and breast cancer in particular. Some of the genes or genomic regions have no direct influence on the members of the ARCHEONs or the genes within distinct chromosomal regions but still retain marker gene function due to their chromosomal positioning in the neighborhood of functionally critical genes (e.g. Telethonin neighboring the Her-2/neu gene).

### Clinical Relevance of the Genes which are Part of the 17q21 Archeon for Response to Herceptin Treatment

Clinical Samples of patients being treated with Herceptin, docetaxel, paclitaxel, taxotere, carboplatin, cisplatin, oxaliplatin, vinorelbine, tamoxifen, anastrozole, letrozole, tamoxifen, epirubicin, doxorubicin and CMF were obtained. Primary tumor tissues and lymphnode tissues were obtained from neoadjuvant and adjuvant settings. In addition, biopsy material of first and second line therapies was obtained in some cases from metastatic lesions. These samples included formalin-fixed and paraffin-embedded material or fresh tissue from primary tumours and metastatic lesions of the respective patients. Moreover, whole blood, serum and plasma samples were included in the analysis.

Multiparametric, clinical assessment of the response to Herceptin in combination with chemotherapeutics (e.g. docetaxel, taxotere, paclitaxel, vinorelbine, carboplatin, cisplatin), or other therapies described below, was performed, based on clinical information, such as histological parameters (TNM-Stage, AJCC grade), standard molecular markers (IHC staining for estrogen receptor, progesteron receptor, Her-2/neu) and sonographical or radiological assessment (e.g. CT). In addition to combinatorial treatment, samples from single agent therapies were evaluated. Response to treatment was evaluated according to international standards. The ARCHEON genes were analyzed on DNA, RNA or protein level. Normalization of the ARCHEON genes was done by intra- or extracbromosomal reference genes (see EXAMPLE 3 below) or by housekeeping genes of diverse expression level.

We could delineate specific regions of the ARCHEON to be informative for the response to a Herceptin-based therapy. As depicted below, genes that are located towards the centromer or telomer of an individual chromosome in relation to a centrally localized gene within an ARCHEON (e.g. Her-2/neu in the 17q21 ARCHEON) are in the following named to be “centromeric” and “telomeric”, respectively. Of particular interest for response to Herceptin-based treatment are genes being centromeric from the Her-2/neu gene locus on 17q21. The integrity of this centromeric ARCHEON region is of importance for the phenotype of Her-2/neu positive tumors. Genetic alteration in the chromosomal region of PIP5KB, FLJ20291, MLN50, TEM7, CACNB1, RPL19, MGC15482, PPARBP, CrkRS are critical for clinical outcome of Her-2/neu positive breast tumors. Of particular interest is the centromeric breakpoint region of the 17q21 ARCHEON nearby the genes TEM7, CACNB1, CrkRS and PPARBP. Her-2/neu positive tumors bearing elevated gene copy numbers of TEM7, CACNB1, CrkRS and PPARBP compared to other Her-2/neu positive tumors and/or normal tissue controls do have a worsened clinical outcome and a poor response to Herceptin based treatment. The genes within this region are involved in calcium and inositol signalling, which is fundamental with regard to cell survival mechanisms (e.g. CACNB1, PPP1R1B and PIP5K2B). Overexpression of CrkRS is of importance for the tumor phenotype, as its kinase activity regulates the RNA polymerase II holoenzyme complex. Especially the phosphorylation of the C-terminal domain and its associated components not only has influence on the general activity of the enzyme complex, but also affects gene products, whose importance for tumor cell growth has been demonstrated and some of which are part of the ARCHEONs (e.g. the SWI/SNF components SMARCs, e.g. SMARCC2, are critical for RB mediated tumor suppression). Phosphorylation of SMARCs is tightly regulated during cell cycle progression and affects the biological, function of the SMARCs (influence on activity, stability and cellular localization). Altered phosphorylation of the RNA polymerase holoenzyme complex by CrkRS therefore most probably affects cell cycle progression. Moreover, the abnormal expression of TEM7, which we found to be elevated in a subclass of Her-2/neu positive tumors, whereas it was originally identified to be a tumor endothelial marker (TEM; see above), points towards an intense interplay between tumor and endothelial cells resulting in a more aggressive behaviour of the respective tumor cells during metastasic processes such as intra- and extravasation. Strikingly, the genes within this region, i.e. ZNF144, TEM7, PIP5K, PPP1R1B and CACNB1, all do have physiological functions within the central nervous system. Therefore, we do assume, that a “neuronal environment” would be favourable for tumor cells overexpressing these genes resulting in growth and survival advantages for these particular tumor cells. In accordance with this, it is observed that Herceptin resistant metastasis frequently occur in the brain. So far it has been discussed, that this observation refers to toxicological problems such as drug-biovailability with respect to the blood brain barrier. It is part of this invention, that genes which are normally expressed within neuronal cells are integral part of the centromeric gene cluster of the ARCHEON on chromosome 17q21 and are involved in de novo and acquired resistance to Herceptin based treatment. Independent amplification units and/or deletion of singular genes of this centromeric cluster due to chromosomal breakage interferes with the survival and resistance function of this genomic region. Therefore the continuity of amplification units is another important feature with regard to responsivenessor unresponsiveness to therapy. It is noteworthy to mention, that not only the presence of particular genes, but also the presence of regulatory elements within this genomic region contribute to the above mentioned biological phenotype. Therefore also the loss or gain of regulatory elements within the centromeric part of the ARCHEON is of importance for resistance to anti cancer treatment and therefore part of this invention.

In addition to the alteration of centromeric ARCHEON region, the total length of the ARCHEON with regard to the telomeric region and the relative gene copy numbers of the amplified genes are of importance. Particularly the integrity of the genomic region harboring the TOP2alpha gene with the surrounding genes THRA, NR1D1, MLN51, WIRE, HsCDC6, RARA, CTEN, IGFBP4, EBI1 and SMARCE1 is of interest. Her-2/neu positive tumors, that are deleted in at least some of this genes exhibit a worsened response to herceptin-based chemotherapy. This demonstrates, that this region is not only of prognostic value for anthracyclin-based therapy, but also of prognostic value for chemotherapeutic treatment with taxol-related agents and platin salts. The amplification, deletion or silencing of this telomeric region is accompanied with altered sensitivity to the above mentioned chemotherapeutics. This is a general feature of tumors bearing alterations (with regard to gene expression and/or amplification of the 17q21 ARCHEON) and not only true for breast cancer. In line with this, we have analyzed ovarian tumors bearing alterations in the 17q21 ARCHEON and correlated the clinical outcome, that was assessed similarly as depicted above, with regard to a platin salt based therapy. Strikingly, tumors with defined genetic patterns within this telomeric regions did develop resistance to this chemotherapeutic regimen. Detecting solely the coamplification of Her-2/neu and TOP2alpha was not as informative with regard to response prediction as a detailed characterization and subsequent response correlation with the region of the THRA, NR1D1, MLN51, WIRE, HsCDC6 and RARA genes. It is part of this invention, that the proliferation status of tumors is affected by genes within ARCHEON regions. The 17q21 ARCHEON determines to at least some extent the proliferation rate of tumor cells. Interestingly, Her-2/neu positive tumors bearing elevated levels of a more limited number of genes, excluding several genes in the telomeric region (i.e. TOP2alpha, HsCDC6) exhibit a relatively slow growth rate, which diminishes the effect of chemotherapeutic drugs targeting proliferating cells and is one of the reasons for the resistance of these tumors to said agents. Instead, these tumors have a higher capacity with regard to invasiveness and do have a diminished apoptotic rate, which to some extent refers to the signaling of Her-2/neu via GRB7 and AKT kinase (also affected by inositols and calcium, see above), respectively. Several genes within the telomeric region of the ARCHEONs affect Her-2/neu signalling, such as RARA, THRA, IGFBP4, and alter the respective characteristics of the cells including proliferation status.

The ARCHEONs being part of this invention, are not only important for clinical response of tumors to antibody-based therapies raised against EGFR- and Her-2/neu signaling (e.g. Herceptin, 2C4 or cetuximab regimen) and to chemotherapeutic agents, but also are of importance for diverse strategies of anti hormonal treatment (e.g. Tamoxifen, Raloxifen, anastrozol, letrozol, faslodex). In particular, elevated levels of the PPARBP gene and protein and the integrity of the telomeric hormone receptor region of the 17q21 ARCHEON, bearing THRA, NR1D1 and RARA, or its related regions on the other ARCHEONs are of importance for these therapeutic regimens. In a retrospective, clinical study evaluating the above mentioned clinical parameters for adjuvant treatment of breast cancer with tamoxifen, we did observe, that the overexpression of PPARBP has impact on the overall survival of patients receiving this therapy. Overexpression of PPARBP enables activity of estrogen and progesteron receptors irrespective of a bound ligand. Therefore, the deregulation of the PPARBP results in the activity of these hormone receptors in the absence of the hormones and even in the presence of anti-hormones and thereby circumvents the anti tumor effect of anti hormonal strategies resulting in resistance of PPARBP overexpressing cells. In addition overexpression of hormone receptors other than estrogen receptor in tumor cells affects activity of estrogen or the respective anti-hormones by competition for dimerization partners, such as RXR, or transcriptional activator or repressor genes, such as CBP or NCOR. With regard to tamoxifen treatment this clearly diminishes the effect of the anti-hormone, as the pool of the transcriptional cofactors is reduced for the classical mode of action of tamoxifen within the nucleus.

The invention further relates to the use of:
A) a polynucleotide comprising at least one of the sequences of SEQ ID NO: 1 to 26 or 53 to 75;
B) a polynucleotide which hybridizes under stringent conditions to a polynucleotide specified in (a) encoding a polypeptide exhibiting the same biological function as specified for the respective sequence in Table 2 or 3
C) a polynucleotide the sequence of which deviates from the polynucleotide specified in (a) and (b) due to the generation of the genetic code encoding a polypeptide exhibiting the same biological function as specified for the respective sequence in Table 2 or 3
D) a polynucleotide which represents a specific fragment, derivative or allelic variation of a polynucleotide sequence specified in (a) to (c)
E) an antisense molecule targeting specifically one of the polynucleotide sequences specified in (a) to (d);
F) a purified polypeptide encoded by a polynucleotide sequence specified in (a) to (d)
G) a purified polypeptide comprising at least one of the sequences of SEQ ID NO: 27 to 52 or 76 to 98;
H) an antibody capable of binding to one of the polynucleotide specified in (a) to (d) or a polypeptide specified in (f) and (g)
I) a reagent identified by any of the methods of claim 14 to 16 that modulates the amount or activity of a polynucleotide sequence specified in (a) to (d) or a polypeptide specified in (f) and (g)

In the preparation of a composition for the prevention, prediction, diagnosis, prognosis or a medicament for the treatment of malignant neoplasia and breast cancer in particular.

### Polynucleotides

A “BREAST CANCER GENE” polynucleotide can be single- or double-stranded and comprises a coding sequence or the complement of a coding sequence for a “BREAST CANCER GENE” polypeptide. Degenerate nucleotide sequences encoding human “BREAST CANCER GENE” polypeptides, as well as homologous nucleotide sequences which are at least about 50, 55, 60, 65, 70, preferably about 75, 90, 96, or 98% identical to the nucleotide sequences of SEQ ID NO: 1 to 26 or 53 to 75 also are “BREAST CANCER GENE” polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologues, and variants of “BREAST CANCER GENE” polynucleotides which encode biologically active “BREAST CANCER GENE” polypeptides also are “BREAST CANCER GENE” polynucleotides.

### Preparation of Polynucleotides

A naturally occurring “BREAST CANCER GENE” polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art.

Any such technique for obtaining a polynucleotide can be used to obtain isolated “BREAST CANCER GENE” polynucleotides. For example, restriction enzymes and probes can be used to isolate polynucleotide fragments which comprises “BREAST CANCER GENE” nucleotide sequences. Isolated polynucleotides are in preparations which are free or at least 70, 80, or 90% free of other molecules.

“BREAST CANCER GENE” cDNA molecules can be made with standard molecular biology techniques, using “BREAST CANCER GENE” mRNA as a template. Any RNA isolation technique which does not select against the isolation of mRNA may be utilized for the purification of such RNA samples. See, for example, Sambrook et al., 1989, (77); and Ausubel, F. M. et al., 1989, (78), both of which are incorporated herein by reference in their entirety. Additionally, large numbers of tissue samples may readily be processed using techniques well known to those of skill in the art, such as, for example, the single-step RNA isolation process of Chomczynski, P. (1989, U.S. Pat. No. 4,843,155), which is incorporated herein by reference in its entirety.

“BREAST CANCER GENE” cDNA molecules can thereafter be replicated using molecular biology techniques known in the art and disclosed in manuals such as Sambrook et al., 1989, (77). An amplification technique, such as PCR, can be used to obtain additional copies of polynucleotides of the invention, using either human genomic DNA or cDNA as a template.

Alternatively, synthetic chemistry techniques can be used to synthesizes “BREAST CANCER GENE” polynucleotides. The degeneracy of the genetic code allows alternate nucleotide sequences to be synthesized which will encode a “BREAST CANCER GENE” polypeptide or a biologically active variant thereof.

### Identification of Differential Expression

Transcripts within the collected RNA samples which represent RNA produced by differentially expressed genes may be identified by utilizing a variety of methods which are ell known to those of skill in the art. For example, differential screening [Tedder, T. F. et al., 1988, (79)], subtractive hybridization [Hedrick, S. M. et al., 1984, (80); Lee, S. W. et al., 1984, (81)], and, preferably, differential display (Liang, P., and Pardee, A. B., 1993, U.S. Pat. No. 5,262,311, which is incorporated herein by reference in its entirety), may be utilized to identify polynucleotide sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the isolated mRNA, and the removal of all hybridized, and therefore double-stranded, sequences. The remaining non-hybridized, single-stranded cDNAs, potentially represent clones derived from genes that are differentially expressed in the two mRNA sources. Such single-stranded cDNAs are then used as the starting material for the construction of a library comprising clones derived from differentially expressed genes.

The differential display technique describes a procedure, utilizing the well known polymerase chain reaction (PCR; the experimental embodiment set forth in Mullis, K. B., 1987, U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times. The pattern of clones resulting from the reverse transcription and amplification of the mRNA of two different cell types is displayed via sequencing gel electrophoresis and compared. Differences in the two banding patterns indicate potentially differentially expressed genes.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ nontranscribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer, ABI), and the entire process from loading of samples to computer analysis and electronic data display can be computer controlled. Capillary electrophoresis is especially preferable for the sequencing of small pieces of DNA which might be present in limited amounts in a particular sample.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR. Upon corroboration, the differentially expressed genes may be further characterized, and may be identified as target and/or marker genes, as discussed, below.

Also, amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

An analysis of the tissue distribution of the mRNA produced by the identified genes may be conducted, utilizing standard techniques well known to those of skill in the art. Such techniques may include, for example, Northern analyses and RT-PCR. Such analyses provide information as to whether the identified genes are expressed in tissues expected to contribute to breast cancer. Such analyses may also provide quantitative information regarding steady state mRNA regulation, yielding data concerning which of the identified genes exhibits a high level of regulation in, preferably, tissues which may be expected to contribute to breast cancer.

Such analyses may also be performed on an isolated cell population of a particular cell type derived from a given tissue. Additionally, standard in situ hybridization techniques may be utilized to provide information regarding which cells within a given tissue express the identified gene. Such analyses may provide information regarding the biological function of an identified gene relative to breast cancer in instances wherein only a subset of the cells within the tissue is thought to be relevant to breast cancer.

Identification of Co-Amplified Genes

Genes involved in genomic alterations (amplifications, insertions, translocations, deletions, etc.) are identified by PCR-based karyotyping in combination with database analysis. Of particular interest are gene amplifications, which account for gene copy numbers >2 per cell. Gene copy number and gene expression of the respective genes often correlates. Therefore clusters of genes being simultaneously overexpressed due to gene amplifications can be identified by expression analysis via DNA-chip technologies or quantitative RTPCR. For example, the altered expression of genes due to increased or decreased gene copy numbers can be determined by GeneArray technologies from Affymetrix or qRT-PCR with the TaqMan or iCycler Systems. Moreover combination of RNA with DNA analytic enables highly parallel and automated characterization of multiple genomic regions of variable length with high resolution in tissue or single cell samples. Furthermore these assays enable the correlation of gene transcription relative to gene copy number of target genes. As there is not necessarily a linear correlation of expression level and gene copy number and as there are synergistic or antagonistic effects in certain gene clusters, the identification on the RNA-level is easier and probably more relevant for the biological outcome of the alterations especially in tumor tissue.

### Detection of Co-Amplified Genes in Malignant Neoplasia

Chromosomal changes are commonly detected by FISH (=Fluorescence-In-Situ-Hybridization) and CGH (=Comparative Genomic Hybridization). For quantification of genomic regions genes or intergenic regions can be used. Such quantification measures the relative abundance of multiple genes with respect to each other (e.g. target gene vs. centromeric region or housekeeping genes). Changes in relative abundance can be detected in paraffin-embedded material even after extraction of RNA or genomic DNA. Measurement of genomic DNA has advantages compared to RNA-analysis due to the stability of DNA, which accounts for the possibility to perform also retrospective studies and offers multiple internal controls (genes not being altered, amplified or deleted) for standardization and exact calculations. Moreover, PCR-analysis of genomic DNA offers the advantage to investigate intergenic, highly variable regions or combinations of SNP's (=Single Nucleotide Polymorphisms), RFLPs, VNTRs and STRs (in general polymorphic markers). Determination of SNPs or polypmorphic markers within defined genomic regions (e.g. SNP analysis by “Pyrosequencing™”) has impact on the phenotype of the genomic alterations. For example it is of advantage to determine combinations of polymorphisms or haplotypes in order to characterize the biological potential of genes being part of amplified alleles. Of particular interest are polymorphic markers in breakpoint regions, coding regions or regulatory regions of genes or intergenic regions. By determining predictive haplotypes with defined biological or clinical outcome it is possible to establish diagnostic and prognostic assays with non-tumor samples from patients. Depending on whether preferably one allele or both alleles to same extent are amplified (=linear or non-linear amplifications) haplotypes can be determined. Overrepresentation of specific polymorphic markers combinations in cells or tissues with gene amplifications facilitates haplotype determination, as e.g. combinations of heterozygous polymorphic markers in nucleic acids isolated from normal tissues, body fluids or biological samples of one patient become almost homozygous in neoplastic tissue of the very same patient. This “gain of homozygosity” corresponds to the measurement of altered genomic region due to amplification events and is suitable for identification of “gain of function”—alterations in tumors, which result in e.g. oncogenic or growth promoting activities. In contrast, the detection of “losses of heterozygosity” is used for identification of anti-oncogenes, gate keeper genes or checkpoint genes, that suppress oncogenic activities and negatively regulate cellular growth processes. This intrinsic difference clearly opposes the impact of the respective genomic regions for tumor development and emphasizes the significance of “gain of homozygosity” measurements disclosed in this invention. In addition to the analyses on SNPs, a comparative approach of blood leucocyte DNA and tumor DNA based on VNTR detection can reveal the existance of a formerly described ARCHEON. SNP and VNTR sequences and primer sets most suitable for detection of the ARCHEON at 17q11-21 are disclosed in Table 4 and Table 6. Detection, quantification and sizing of such polymorphic markers can be achieved by methods known to those with skill in the art. In one embodiment of this invention we disclose the comparative measurement of amount and size of any of the disclosed VNTRs (Table 6) by PCR amplification and capillary electrophoresis. PCR can be carried out by standart protocols favorably in a linear amplification range (low cycle number) and detection by CE should be carried out by suppliers protocols (e.g. Agilent). More favorably the detection of the VNTRs disclosed in Table 6 can be carried out in a multiplex fashion, utilizing a variety of labeled primers (e.g. fluorescent, radioactive, bioactive) and a suitable CE detection system (e.g. ABI 310). However the detection can also be performed on slab gels consisting of highly concentrated agarose or polyacrylamide with a monochromal DNA stain. Enhancement of resolution can be achieved by appropriate primer design and length variation to give best results in multiplex PCR.

It is also of interest to determine covalent modifications of DNA (e.g. methylation) or the associated chromatin (e.g. acetylation or methylation of associated proteins) within the altered genomic regions, that have impact on transcriptional activity of the genes. In general, by measuring multiple, short sequences (60-300 bp) these techniques enable high-resolution analysis of target regions, which cannot be obtained by conventional methods such as FISH analytic (2-100 kb). Moreover the PCR-based DNA analysis techniques offer advantages with regard to sensitivity, specificity, multiplexing, time consumption and low amount of patient material required. These techniques can be optimized by combination with microdissection or macrodissection to obtain purer starting material for analysis.

### Extending Polynucleotides

In one embodiment of such a procedure for the identification and cloning of full length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA hybrid may then be “tailed” with guanines using a standard terminal transferase reaction, the hybrid may be digested with RNase H, and second strand synthesis may then be primed with a poly-C primer. Using the two primers, the 5′ portion of the gene is amplified using PCR Sequences obtained may then be isolated and recombined with previously isolated sequences to generate a full-length cDNA of the differentially expressed genes of the invention. For a review of cloning strategies and recombinant DNA techniques, see e.g., Sambrook et al., (77); and Ausubel et al., (78).

Various PCR-based methods can be used to extend the polynucleotide sequences disclosed herein to detect upstream sequences such as promoters and regulatory elements. For example, restriction site PCR uses universal primers to retrieve unknown sequence adjacent to a known locus [Sarkar, 1993, (82)]. Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region [Triglia et al., 1988, (83)]. Primers can be designed using commercially available software, such as OLIGO 4.06 Primer Analysis software (National Biosciences Inc., Plymouth, Minn.), to be e.g. 2230 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA [Lagerstrom et al., 1991, (84)]. In this method, multiple restriction enzyme digestions and ligations also can be used to place an engineered double-stranded sequence into an unknown fragment of the DNA molecule before performing PCR.

Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

The sequences of the identified genes may be used, utilizing standard techniques, to place the genes onto genetic maps, e.g., mouse [Copeland & Jenkins, 1991, (85)] and human genetic maps [Cohen, et al., 1993, (86)]. Such mapping information may yield information regarding the genes' importance to human disease by, for example, identifying genes which map near genetic regions to which known genetic breast cancer tendencies map.

### Identification of Polynucleotide Variants and Homologues or Splice Variants

Variants and homologues of the “BREAST CANCER GENE” polynucleotides described above also are “BREAST CANCER GENE” polynucleotides. Typically, homologous “BREAST CANCER GENE” polynucleotide sequences can be identified by hybridization of candidate polynucleotides to known “BREAST CANCER GENE” polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50 EC once, 30 minutes; then 2×SSC, room temperature twice, 10 minutes each homologous sequences can be identified which contain at most about 25-30% basepair mismatches. More preferably, homologous polynucleotide strands contain 15-25% basepair mismatches, even more preferably 5-15% basepair mismatches.

Species homologues of the “BREAST CANCER GENE” polynucleotides disclosed herein also can be identified by making suitable probes or primers and screening cDNA expression libraries from other species, such as mice, monkeys, or yeast. Human variants of “BREAST CANCER GENE” polynucleotides can be identified, for example, by screening human cDNA expression libraries. It is well known that the Tₘ of a double-stranded DNA decreases by 1-1.5° C. with every 1% decrease in homology [Bonner et al., 1973, (87)]. Variants of human “BREAST CANCER GENE” polynucleotides or “BREAST CANCER GENE” polynucleotides of other species can therefore be identified by hybridizing a putative homologous “BREAST CANCER GENE” polynucleotide with a polynucleotide having a nucleotide sequence of one of the sequences of the SEQ ID NO: 1 to 26 or 53 to 75 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to “BREAST CANCER GENE” polynucleotides or their complements following stringent hybridization and/or wash conditions also are “BREAST CANCER GENE” polynucleotides. Stringent wash conditions are well known and understood in the art and are disclosed, for example, in Sambrook et al., (77). Typically, for stringent hybridization conditions a combination of temperature and salt concentration should be chosen that is approximately 12-20° C. below the calculated Tₘ of the hybrid under study. The Tₘ of a hybrid between a “BREAST CANCER GENE” polynucleotide having a nucleotide sequence of one of the sequences of the SEQ ID NO: 1 to 26 or 53 to 75 or the complement thereof and a polynucleotide sequence which is at least about 50, preferably about 75, 90, 96, or 98% identical to one of those nucleotide sequences can be calculated, for example, using the equation below [Bolton and McCarthy, 1962, (88):

Tₘ=81.5° C.−16.6(log₁₀[Na⁺])+0.41(% G+C)−0.63(% formamide)−600/l),
where l=the length of the hybrid in basepairs.

Stringent wash conditions include, for example, 4×SSC at 65° C., or 50% formamide, 4×SSC at 28° C., or 0.5×SSC, 0.1% SDS at 65° C. Highly stringent wash conditions include, for example, 0.2×SSC at 65° C.

The biological function of the identified genes may be more directly assessed by utilizing relevant in vivo and in vitro systems. In vivo systems may include, but are not limited to, animal systems which naturally exhibit breast cancer predisposition, or ones which have been engineered to exhibit such symptoms, including but not limited to the apoE-deficient malignant neoplasia mouse model [Plump et al., 1992, (89)].

Splice variants derived from the same genomic region, encoded by the same pre mRNA can be identified by hybridization conditions described above for homology search. The specific characteristics of variant proteins encoded by splice variants of the same pre transcript may differ and can also be assayed as disclosed. A “BREAST CANCER GENE” polynucleotide having a nucleotide sequence of one of the sequences of the SEQ ID NO: 1 to 26 or 53 to 75 or the complement thereof may therefor differ in parts of the entire sequence as presented for SEQ ID NO: 60 and the encoded splice variants SEQ ID NO: 61 to 66. These refer to individual proteins SEQ ID NO: 83 to 89. The prediction of splicing events and the identification of the utilized acceptor and donor sites within the pre mRNA can be computed (e.g. Software Package GRAIL or GenomeSCAN) and verified by PCR method by those with skill in the art.

### Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 6 nucleotides in length, but can be at least 7, 8, 10, 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of “BREAST CANCER GENE” gene products in the cell.

Antisense oligonucleotides can be deoxyribonucleotides, ribonucleotides, peptide nucleic acids (PNAs; described in U.S. Pat. No. 5,714,331), locked nucleic acids (LNAs; described in WO 99/12826), or a combination of them. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters [Brown, 1994, (126); Sonveaux, 1994, (127) and Uhlmann et al., 1990, (128)].

Modifications of “BREAST CANCER GENE” expression can be obtained by designing antisense oligonucleotides which will form duplexes to the control, 5′, or regulatory regions of the “BREAST CANCER GENE”. Oligonucleotides derived from the transcription initiation site, e.g., between positions 10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature [Gee et al., 1994, (129)]. An antisense oligonucleotide also can be designed to block translation of mRNA by preventing the transcript from binding to ribosomes.

Precise complementarity is not required for successful complex formation between an antisense oligonucleotide and the complementary sequence of a “BREAST CANCER GENE” polynucleotide. Antisense oligonucleotides which comprise, for example, 2, 3, 4, or 5 or more stretches of contiguous nucleotides which are precisely complementary to a “BREAST CANCER GENE” polynucleotide, each separated by a stretch of contiguous nucleotides which are not complementary to adjacent “BREAST CANCER GENE” nucleotides, can provide sufficient targeting specificity for “BREAST CANCER GENE” mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular “BREAST CANCER GENE” polynucleotide sequence.

Antisense oligonucleotides can be modified without affecting their ability to hybridize to a “BREAST CANCER GENE” polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′ substituted oligonucleotide in which the 3′ hydroxyl group or the 5′ phosphate group are substituted, also can be employed in a modified antisense oligonucleotide. These modified oligonucleotides can be prepared by methods well known in the art [Agrawal et al., 1992, (130); Uhlmann et al., 1987, (131) and Uhlmann et al., (128)].

### Ribozymes

Ribozymes are RNA molecules with catalytic activity [Cech, 1987, (132); Cech, 1990, (133) and Couture & Stinchcomb, 1996, (134)]. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The transcribed sequence of a “BREAST CANCER GENE” can be used to generate ribozymes which will specifically bind to mRNA transcribed from a “BREAST CANCER GENE” genomic locus. Methods of designing and constructing ribozymes which can cleave other RNA molecules in trans in a highly sequence specific manner have been developed and described in the art [Haseloff et al., 1988, (135)]. For example, the cleavage activity of ribozymes can be targeted to specific RNAs by engineering a discrete “hybridization” region into the ribozyme. The hybridization region contains a sequence complementary to the target RNA and thus specifically hybridizes with the target [see, for example, Gerlach et al., EP 0 321201].

Specific ribozyme cleavage sites within a “BREAST CANCER GENE” RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate “BREAST CANCER GENE” RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease “BREAST CANCER GENE” expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a nbozyme and a target gene are induced in the cells.

### Polypeptides

“BREAST CANCER GENE” polypeptides according to the invention comprise an polypeptide selected from SEQ ID NO: 27 to 52 and 76 to 98 or encoded by any of the polynucleotide sequences of the SEQ ID NO: 1 to 26 and 53 to 75 or derivatives, fragments, analogues and homologues thereof. A “BREAST CANCER GENE” polypeptide of the invention therefore can be a portion, a full-length, or a fusion protein comprising all or a portion of a “BREAST CANCER GENE” polypeptide.

### Protein Purification

“BREAST CANCER GENE” polypeptides can be purified from any cell which expresses the enzyme, including host cells which have been transfected with “BREAST CANCER GENE” expression constructs. Breast tissue is an especially useful source of “BREAST CANCER GENE” polypeptides. A purified “BREAST CANCER GENE” polypeptide is separated from other compounds which normally associate with the “BREAST CANCER GENE” polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified “BREAST CANCER GENE” polypeptides is at least 80% pure; preferably, the preparations are 90%, 95%, or 99% pure. Purity of the preparations can be assessed by any means known in the art, such as SDS-polyacrylamide gel electrophoresis.

### Obtaining Polypeptides

“BREAST CANCER GENE” polypeptides can be obtained, for example, by purification from human cells, by expression of “BREAST CANCER GENE” polynucleotides, or by direct chemical synthesis.

### Biologically Active Variants

“BREAST CANCER GENE” polypeptide variants which are biologically active, i.e., retain an “BREAST CANCER GENE” activity, also are “BREAST CANCER GENE” polypeptides. Preferably, naturally or non-naturally occurring “BREAST CANCER GENE” polypeptide variants have amino acid sequences which are at least about 60, 65, or 70, preferably about 75, 80, 85, 90, 92, 94, 96, or 98% identical to the any of the amino acid sequences of the polypeptides of SEQ ID NO: 27 to 52 or 76 to 98 or the polypeptides encoded by any of the polynucleotides of SEQ ID NO: 1 to 26 or 53 to 75 or a fragment thereof. Percent identity between a putative “BREAST CANCER GENE” polypeptide variant and of the polypeptides of SEQ ID NO: 27 to 52 or 76 to 98 or the polypeptides encoded by any of the polynucleotides of SEQ ID NO: 1 to 26 or 53 to 75 or a fragment thereof is determined by conventional methods. [See, for example, Altschul et al., 1986, (90 and Henikoff & Henikoff, 1992, (91)]. Briefly, two amino acid sequences are aligned to optimize the alignment scores using a gap opening penalty of 10, a gap extension penalty of 1, and the “BLOSUM62” scoring matrix of Henikoff & Henikoff, (91).

Those skilled in the art appreciate that there are many established algorithms available to align two amino acid sequences. The “FASTA” similarity search algorithm of Pearson & Lipman is a suitable protein alignment method for examining the level of identity shared by an amino acid sequence disclosed herein and the amino acid sequence of a putative variant [Pearson & Lipman, 1988, (92), and Pearson, 1990, (93)]. Briefly, FASTA first characterizes sequence similarity by identifying regions shared by the query sequence (e.g., SEQ ID NO: 1 to 26 or 53 to 75) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm [Needleman & Wunsch, 1970, (94), and Sellers, 1974, (95)], which allows for amino acid insertions and deletions. Preferred parameters for FASTA analysis are: ktup=1, gap opening penalty=10, gap extension penalty=1, and substitution matrix-BLOSUM62. These parameters can be introduced into a FASTA program by modifying the scoring matrix file (“SMATRIX”), as explained in Appendix 2 of Pearson, (93).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a “BREAST CANCER GENE” polypeptide can be found using computer programs well known in the art, such as DNASTAR software. Whether an amino acid change results in a biologically active “BREAST CANCER GENE” polypeptide can readily be determined by assaying for “BREAST CANCER GENE” activity, as described for example, in the specific Examples, below. Larger insertions or deletions can also be caused by alternative splicing. Protein domains can be inserted or deleted without altering the main activity of the protein.

### Fusion Proteins

Fusion proteins are useful for generating antibodies against “BREAST CANCER GENE” polypeptide amino acid sequences and for use in various assay systems. For example, fusion proteins can be used to identify proteins which interact with portions of a “BREAST CANCER GENE” polypeptide. Protein affinity chromatography or library-based assays for protein-protein interactions, such as the yeast two-hybrid or phage display systems, can be used for this purpose. Such methods are well known in the art and also can be used as drug screens.

A “BREAST CANCER GENE” polypeptide fusion protein comprises two polypeptide segments fused together by means of a peptide bond. The first polypeptide segment comprises at least 25, 50, 75, 100, 150, 200, 300, 400, 500, 600, 700 or 750 contiguous amino acids of an amino acid sequence encoded by any polynucleotide sequences of the SEQ D NO: 1 to 26 or 53 to 75 or of a biologically active variant, such as those described above. The first polypeptide segment also can comprise full-length “BREAST CANCER GENE”.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP) glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the “BREAST CANCER GENE” polypeptide-encoding sequence and the heterologous protein sequence, so that the “BREAST CANCER GENE” polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from any of the polynucleotide sequences of the SEQ ID NO: 1 to 26 and 53 to 75 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologues

Species homologues of human a “BREAST CANCER GENE” polypeptide can be obtained using “BREAST CANCER GENE” polypeptide polynucleotides (described below) to make suitable probes or primers for screening cDNA expression libraries from other species, such as mice, monkeys, or yeast, identifying cDNAs which encode homologues of a “BREAST CANCER GENE” polypeptide, and expressing the cDNAs as is known in the art.

### Expression of Polynucleotides

To express a “BREAST CANCER GENE” polynucleotide, the polynucleotide can be inserted into an expression vector which contains the necessary elements for the transcription and translation of the inserted coding sequence. Methods which are well known to those skilled in the art can be used to construct expression vectors containing sequences encoding “BREAST CANCER GENE” polypeptides and appropriate transcriptional and translational control elements. These methods include in vitro recombinant DNA techniques, synthetic techniques, and in vivo genetic recombination. Such techniques are described, for example, in Sambrook et al., (77) and in Ausubel et al., (78).

A variety of expression vector/host systems can be utilized to contain and express sequences encoding a “BREAST CANCER GENE” polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those regions of the vector enhancers, promoters, 5′ and 3′ untranslated regions which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a “BREAST CANCER GENE” polypeptide, vectors based on SV40 or EBV can be used with an appropriate selectable marker.

### Bacterial and Yeast Expression Systems

In bacterial systems, a number of expression vectors can be selected depending upon the use intended for the “BREAST CANCER GENE” polypeptide. For example, when a large quantity of the “BREAST CANCER GENE” polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the “BREAST CANCER GENE” polypeptide can be ligated into the vector in frame with sequences for the amino terminal Met and the subsequent 7 residues of β-galactosidase so that a hybrid protein is produced. pIN vectors [Van Heeke & Schuster, (17)] or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al., (4) and Grant et al., (18).

### Plant and Insect Expression Systems

If plant expression vectors are used, the expression of sequences encoding “BREAST CANCER GENE” polypeptides can be driven by any of a number of promoters. For example, viral promoters such as the 35S and 19S promoters of CaMV can be used alone or in combination with the omega leader sequence from TMV [Takamatsu, 1987, (96)]. Alternatively, plant promoters such as the small subunit of RUBISCO or heat shock promoters can be used [Coruzzi et al., 1984, (97); Broglie et al., 1984, (98); Winter et al., 1991, (99)]. These constructs can be introduced into plant cells by direct DNA transformation or by pathogen-mediated transfection. Such techniques are described in a number of generally available reviews.

An insect system also can be used to express a “BREAST CANCER GENE” polypeptide. For example, in one such system Autographa californica nuclear polyhedrosis virus (AcNPV) is used as a vector to express foreign genes in Spodoptera frugiperda cells or in Trichoplusia larvae. Sequences encoding “BREAST CANCER GENE” polypeptides can be cloned into a nonessential region of the virus, such as the polyhedrin gene, and placed under control of the polyhedrin promoter. Successful insertion of “BREAST CANCER GENE” polypeptides will render the polyhedrin gene inactive and produce recombinant virus lacking coat protein. The recombinant viruses can then be used to infect S. frugiperda cells or Trichoplusia larvae in which “BREAST CANCER GENE” polypeptides can be expressed [Engelhard et al., 1994, (100)].

### Mammalian Expression Systems

A number of viral-based expression systems can be used to express “BREAST CANCER GENE” polypeptides in mammalian host cells. For example, if an adenovirus is used as an expression vector, sequences encoding “BREAST CANCER GENE” polypeptides can be ligated into an adenovirus transcription/translation complex comprising the late promoter and tripartite leader sequence. Insertion in a nonessential E1 or E3 region of the viral genome can be used to obtain a viable virus which is capable of expressing a “BREAST CANCER GENE” polypeptide in infected host cells [Logan & Shenk, 1984, (101)]. If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding “BREAST CANCER GENE” polypeptides. Such signals include the ATG initiation codon and adjacent sequences. In cases where sequences encoding a “BREAST CANCER GENE” polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used [Scharf et al., 1994, (102)].

### Host Cells

A host cell strain can be chosen for its ability to modulate the expression of the inserted sequences or to process the expressed “BREAST CANCER GENE” polypeptide in the desired fashion. Such modifications of the polypeptide include, but are not limited to, acetylation, carboxylation, glycosylation, phosphorylation, lipidation, and acylation. Posttranslational processing which cleaves a “prepro” form of the polypeptide also can be used to facilitate correct insertion, folding and/or function. Different host cells which have specific cellular machinery and characteristic mechanisms for Post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express “BREAST CANCER GENE” polypeptides can be transformed using expression vectors which can contain viral origins of replication and/or endogenous expression elements and a selectable marker gene on the same or on a separate vector. Following the introduction of the vector, cells can be allowed to grow for 12 days in an enriched medium before they are switched to a selective medium. The purpose of the selectable marker is to confer resistance to selection, and its presence allows growth and recovery of cells which successfully express the introduced “BREAST CANCER GENE” sequences. Resistant clones of stably transformed cells can be proliferated using tissue culture techniques appropriate to the cell type [Freshney et al., 1986, (103).

Any number of selection systems can be used to recover transformed cell lines. These include, but are not limited to, the herpes simplex virus thymidine kinase (Wigler et al., 1977, (104)] and adenine phosphoribosyltransferase [Lowy et al., 1980, (105)] genes which can be employed in tk⁻ or aprt⁻ cells, respectively. Also, antimetabolite, antibiotic, or herbicide resistance can be used as the basis for selection. For example, dhfr confers resistance to methotrexate [Wigler et al., 1980, (106)], npt confers resistance to the aminoglycosides, neomycin and G418 [Colbere-Garapin et al., 1981, (107)], and als and pat confer resistance to chlorsulfuron and phosphinotricin acetyltransferase, respectively. Additional selectable genes have been described. For example, trpB allows cells to utilize indole in place of tryptophan, or hisD, which allows cells to utilize histinol in place of histidine [Hartman & Mulligan, 1988, (108)]. Visible markers such as anthocyanins, β-glucuronidase and its substrate GUS, and luciferase and its substrate luciferin, can be used to identify transformants and to quantify the amount of transient or stable protein expression attributable to a specific vector system [Rhodes et al., 1995, (109)].

### Detecting Expression and Gene Product

Although the presence of marker gene expression suggests that the “BREAST CANCER GENE” polynucleotide is also present, its presence and expression may need to be confirmed. For example, if a sequence encoding a “BREAST CANCER GENE” polypeptide is inserted within a marker gene sequence, transformed cells containing sequences which encode a “BREAST CANCER GENE” polypeptide can be identified by the absence of marker gene function. Alternatively, a marker gene can be placed in tandem with a sequence encoding a “BREAST CANCER GENE” polypeptide under the control of a single promoter. Expression of the marker gene in response to induction or selection usually indicates expression of the “BREAST CANCER GENE” polynucleotide.

Alternatively, host cells which contain a “BREAST CANCER GENE” polynucleotide and which express a “BREAST CANCER GENE” polypeptide can be identified by a variety of procedures known to those of skill in the art. These procedures include, but are not limited to, DNA-DNA or DNA-RNA hybridization and protein bioassay or immunoassay techniques which include membrane, solution, or chip-based technologies for the detection and/or quantification of polynucleotide or protein. For example, the presence of a polynucleotide sequence encoding a “BREAST CANCER GENE” polypeptide can be detected by DNA-DNA or DNA-RNA hybridization or amplification using probes or fragments or fragments of polynucleotides encoding a “BREAST CANCER GENE” polypeptide. Nucleic acid amplification-based assays involve the use of oligonucleotides selected from sequences encoding a “BREAST CANCER GENE” polypeptide to detect transformants which contain a “BREAST CANCER GENE” polynucleotide.

A variety of protocols for detecting and measuring the expression of a “BREAST CANCER GENE” polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a “BREAST CANCER GENE” polypeptide can be used, or a competitive binding assay can be employed. These and other assays are described in Hampton et al., (110) and Maddox et al., 111).

A wide variety of labels and conjugation techniques are known by those skilled in the art and can be used in various nucleic acid and amino acid assays. Means for producing labeled hybridization or PCR probes for detecting sequences related to polynucleotides encoding “BREAST CANCER GENE” polypeptides include oligo labeling, nick translation, end-labeling, or PCR amplification using a labeled nucleotide. Alternatively, sequences encoding a “BREAST CANCER GENE” polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

### Expression and Purification of Polypeptides

Host cells transformed with nucleotide sequences encoding a “BREAST CANCER GENE” polypeptide can be cultured under conditions suitable for the expression and recovery of the protein from cell culture. The polypeptide produced by a transformed cell can be secreted or stored intracelluar depending on the sequence and/or the vector used. As will be understood by those of skill in the art, expression vectors containing polynucleotides which encode “BREAST CANCER GENE” polypeptides can be designed to contain signal sequences which direct secretion of soluble “BREAST CANCER GENE” polypeptides through a prokaryotic or eukaryotic cell membrane or which direct the membrane insertion of membrane-bound “BREAST CANCER GENE” polypeptide.

As discussed above, other constructions can be used to join a sequence encoding a “BREAST CANCER GENE” polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system Immunex Corp., Seattle, Wash.). Inclusion of cleavable linker sequences such as those specific for Factor Xa or enterokinase (Invitrogen, San Diego, Calif.) between the purification domain and the “BREAST CANCER GENE” polypeptide also can be used to facilitate purification. One such expression vector provides for expression of a fusion protein containing a “BREAST CANCER GENE” polypeptide and 6 histidine residues preceding a thioredoxin or an enterokinase cleavage site. The histidine residues facilitate purification by IMAC (immobilized metal ion affinity chromatography [Porath et al., 1992, (112)], while the enterokinase cleavage site provides a means for purifying the “BREAST CANCER GENE” polypeptide from the fusion protein. Vectors which contain fusion proteins are disclosed in Kroll et al., (113).

### Chemical Synthesis

Sequences encoding a “BREAST CANCER GENE” polypeptide can be synthesized, in whole or in part, using chemical methods well known in the art (see Caruthers et al., (114) and Hom et al., (115). Alternatively, a “BREAST CANCER GENE” polypeptide itself can be produced using chemical methods to synthesize its amino acid sequence, such as by direct peptide synthesis using solid-phase techniques [Merrifield, 1963, (116) and Roberge et al., 1995, (117)]. Protein synthesis can be performed using manual techniques or by automation. Automated synthesis can be achieved, for example, using Applied Biosystems 431A Peptide Synthesizer (Perkin Elner). Optionally, fragments of “BREAST CANCER GENE” polypeptides can be separately synthesized and combined using chemical methods to produce a full-length molecule.

The newly synthesized peptide can be substantially purified by preparative high performance liquid chromatography [Creighton, 1983, (118)]. The composition of a synthetic “BREAST CANCER GENE” polypeptide can be confirmed by amino acid analysis or sequencing (e.g., the Edrnan degradation procedure; see Creighton, (118). Additionally, any portion of the amino acid sequence of the “BREAST CANCER GENE” polypeptide can be altered during direct synthesis and/or combined using chemical methods with sequences from other proteins to produce a variant polypeptide or a fusion protein.

### Production of Altered Polypeptides

As will be understood by those of skill in the art, it may be advantageous to produce “BREAST CANCER GENE” polypeptide-encoding nucleotide sequences possessing non-natural occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter “BREAST CANCER GENE” polypeptide-encoding sequences for a variety of reasons, including but not limited to, alterations which modify the cloning, processing, and/or expression of the polypeptide or mRNA product. DNA shuffling by random fragmentation and PCR re-assembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

### Predictive, Diagnostic and Prognostic Assays

The present invention provides method for determining whether a subject is at risk for developing malignant neoplasia and breast cancer in particular by detecting one of the disclosed polynucleotide markers comprising any of the polynucleotides sequences of the SEQ ID NO: 2 to 6, 8, 9, 11 to 16, 18, 19 or 21 to 26 or 53 to 75 and/or the polypeptide markers encoded thereby or polypeptide markers comprising any of the polypeptide sequences of the SEQ ID NO: 28 to 32, 34, 35, 37 to 42, 44, 45 or 47 to 52 or 76 to 98 or at least 2 of the disclosed polynucleotides selected from SEQ ID NO: 1 to 26 and 53 to 75 or the at least 2 of the disclosed polypeptides selected from SEQ ID NO: 28 to 32 and 76 to 98 for malignant neoplasia and breast cancer in particular.

In clinical applications, biological samples can be screened for the presence and/or absence of the biomarkers identified herein. Such samples are for example needle biopsy cores, surgical resection samples, or body fluids like serum, thin needle nipple aspirates and urine. For example, these methods include obtaining a biopsy, which is optionally fractionated by cryostat sectioning to enrich diseases cells to about 80% of the total cell population. In certain embodiments, polynucleotides extracted from these samples may be amplified using techniques well known in the art. The expression levels of selected markers detected would be compared with statistically valid groups of diseased and healthy samples.

In one embodiment the diagnostic method comprises determining whether a subject has an abnormal mRNA and/or protein level of the disclosed markers, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the levels of the disclosed biomarkers, protein or mRNA level, is determined and compared to the level of these markers in a healthy subject. An abnormal level of the biomarker polypeptide or mRNA levels is likely to be indicative of malignant neoplasia such as breast cancer.

In another embodiment the diagnostic method comprises determining whether a subject has an abnormal DNA content of said genes or said genomic loci, such as by Southern blot analysis, dot blot analysis, fluorescence or colorimetric In Situ hybridization, comparative genomic hybridization, genotpying by VNTR, STS-PCR or quantitative PCR. In general these assays comprise the usage of probes from representative genomic regions. The probes contain at least parts of said genomic regions or sequences complementary or analogous to said regions. In particular intra- or intergenic regions of said genes or genomic regions. The probes can consist of nucleotide sequences or sequences of analogous functions (e.g. PNAs, Morpholino oligomers) being able to bind to target regions by hybridization. In general genomic regions being altered in said patient samples are compared with unaffected control samples (normal tissue from the same or different patients, surrounding unaffected tissue, peripheral blood) or with genomic regions of the same sample that don't have said alterations and can therefore serve as internal controls. In a preferred embodiment regions located on the same chromosome are used. Alternatively, gonosomal regions and/or regions with defined varying amount in the sample are used. In one favored embodiment the DNA content, structure, composition or modification is compared that lie within distinct genomic regions. Especially favored are methods that detect the DNA content of said samples, where the amount of target regions are altered by amplification and or deletions. In another embodiment the target regions are analyzed for the presence of polymorphisms (e.g. Single Nucleotide Polymorphisms or mutations) that affect or predispose the cells in said samples with regard to clinical aspects, being of diagnostic, prognostic or therapeutic value. Preferably, the identification of sequence variations is used to define haplotypes that result in characteristic behavior of said samples with said clinical aspects.

The following examples of genes in 17q12-21.2 are offered by way of illustration, not by way of limitation.

One embodiment of the invention is a method for the prediction, diagnosis or prognosis of malignant neoplasia by the detection of at least 10, at least 5, or at least 4, or at least 3 and more preferably at least 2 markers whereby the markers are genes and fragments thereof and/or genomic nucleic acid sequences that are located on one chromosomal region which is altered in malignant neoplasia.

One further embodiment of the invention is method for the prediction, diagnosis or prognosis of malignant neoplasia by the detection of at least 10, at least 5, or at least 4, or at least 3 and more preferably at least 2 markers whereby the markers (a) are genes and fragments thereof and/or genomic nucleic acid sequences that are located on one or more chromosomal region(s) which is/are altered in malignant neoplasia and (b) functionally interact as (i) receptor and ligand or (ii) members of the same signal transduction pathway or (iii) members of synergistic signal transduction pathways or (iv) members of antagonistic signal transduction pathways or (v) transcription factor and transcription factor binding site.

In one embodiment, the method for the prediction, diagnosis or prognosis of malignant neoplasia and breast cancer in particular is done by the detection of:
(a) polynucleotide selected from the polynucleotides of the SEQ D NO: 2 to 6, 8, 9, 11 to 16, 18, 19, 21 to 26 or 53 to 75;
(b) a polynucleotide which hybridizes under stringent conditions to a polynucleotide specified in (a) encoding a polypeptide exhibiting the same biological function as specified for the respective sequence in Table 2 or 3;
(c) a polynucleotide the sequence of which deviates from the polynucleotide specified in (a) and (b) due to the generation of the genetic code encoding a polypeptide exhibiting the same biological function as specified for the respective sequence in Table 2 or 3;
(d) a polynucleotide which represents a specific fragment, derivative or allelic variation of a polynucleotide sequence specified in (a) to (c);
   in a biological sample comprising the following steps: hybridizing any polynucleotide or analogous oligomer specified in (a) to (do) to a polynucleotide material of a biological sample, thereby forming a hybridization complex; and detecting said hybridization complex.

In another embodiment the method for the prediction, diagnosis or prognosis of malignant neoplasia is done as just described but, wherein before hybridization, the polynucleotide material of the biological sample is amplified.

In another embodiment the method for the diagnosis or prognosis of malignant neoplasia and breast cancer in particular is done by the detection of:
(a) a polynucleotide selected from the polynucleotides of the SEQ ID NO: 2 to 6, 8, 9, 11 to 16, 18, 19, 21 to 26 or 53 to 75;
(b) a polynucleotide which hybridizes under stringent conditions to a polynucleotide specified in (a) encoding a polypeptide exhibiting the same biological function as specified for the respective sequence in Table 2 or 3;
(c) a polynucleotide the sequence of which deviates from the polynucleotide specified in (a) and (b) due to the generation of the genetic code encoding a polypeptide exhibiting the same biological function as specified for the respective sequence in Table 2 or 3;
(d) a polynucleotide which represents a specific fragment, derivative or allelic variation of a polynucleotide sequence specified in (a) to (c);
(e) a polypeptide encoded by a polynucleotide sequence specified in (a) to (d)
(f) a polypeptide comprising any polypeptide of SEQ ID NO: 28 to 32, 34, 35, 37 to 42, 44, 45, 47 to 52 or 76 to 98;
   comprising the steps of contacting a biological sample with a reagent which specifically interacts with the polynucleotide specified in (a) to (d) or the polypeptide specified in (e).

### DNA Array Technology

In one embodiment, the present Invention also provides a method wherein polynucleotide probes are immobilized an a DNA chip in an organized array. Oligonucleotides can be bound to a solid Support by a variety of processes, including lithography. For example a chip can hold up to 4100,00 oligonucleotides (GeneChip, Affymetrix). The present invention provides significant advantages over the available tests for malignant neoplasia, such as breast cancer, because it increases the reliability of the test by providing an array of polynucleotide markers an a single chip.

The method includes obtaining a biopsy of an affected person, which is optionally fractionated by cryostat sectioning to enrich diseased cells to about 80% of the total cell population and the use of body fluids such as serum or urine, serum or cell containing liquids (e.g. derived from fine needle aspirates). The DNA or RNA is then extracted, amplified, and analyzed with a DNA chip to determine the presence of absence of the marker polynucleotide sequences. In one embodiment, the polynucleotide probes are spotted onto a substrate in a two-dimensional matrix or array. samples of polynucleotides can be labeled and then hybridized to the probes. Double-stranded polynucleotides, comprising the labeled sample polynucleotides bound to probe polynucleotides, can be detected once the unbound portion of the sample is washed away.

The probe polynucleotides can be spotted an substrates including glass, nitrocellulose, etc. The probes can be bound to the Substrate by either covalent bonds or by non-specific interactions, such as hydrophobic interactions. The sample polynucleotides can be labeled using radioactive labels, fluorophores, chromophores, etc. Techniques for constructing arrays and methods of using these arrays are described in EP 0 799 897; WO 97/29212; WO 97/27317; EP 0 785 280; WO 97/02357; U.S. Pat. No. 5,593,839; U.S. Pat. No. 5,578,832; EP 0 728 520; U.S. Pat. No. 5,599,695; EP 0 721 016; U.S. Pat. No. 5,556,752; WO 95/22058; and U.S. Pat. No. 5,631,734. Further, arrays can be used to examine differential expression of genes and can be used to determine gene function. For example, arrays of the instant polynucleotide sequences can be used to determine if any of the polynucleotide sequences are differentially expressed between normal cells and diseased cells, for example. High expression of a particular message in a diseased sample, which is not observed in a corresponding normal sample, can indicate a breast cancer specific protein.

Accordingly, in one aspect, the invention provides probes and primers that are specific to the unique polynucleotide markers disclosed herein.

In one embodiment, the method comprises using a polynucleotide probe to determine the presence of malignant or breast cancer cells in particular in a tissue from a patient. Specifically, the method comprises:
1) providing a polynucleotide probe comprising a nucleotide sequence at least 12 nucleotides in length, preferably at least 15 nucleotides, more preferably, 25 nucleotides, and most preferably at least 40 nucleotides, and up to all or nearly all of the coding sequence which is complementary to a portion of the coding sequence of a polynucleotide selected from the polynucleotides of SEQ ID NO: 1 to 26 and 53 to 75 or a sequence complementary thereto and is
2) differentially expressed in malignant neoplasia, such as breast cancer;
3) obtaining a tissue sample from a patient with malignant neoplasia;
4) providing a second tissue sample from a patient with no malignant neoplasia;
5) contacting the polynucleotide probe under stringent conditions with RNA of each of said first and second tissue samples (e.g., in a Northern blot or in situ hybridization assay); and
6) comparing (a) the amount of hybridization of the probe with RNA of the first tissue sample, with (b) the amount of hybridization of the probe with RNA of the second tissue sample;
   wherein a statistically significant difference in the amount of hybridization with the RNA of the first tissue sample as compared to the amount of hybridization with the RNA of the second tissue sample is indicative of malignant neoplasia and breast cancer in particular in the first tissue sample.

### Data Analysis Methods

Comparison of the expression levels of one or more “BREAST CANCER GENES” with reference expression levels, e.g., expression levels in diseased cells of breast cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one “BREAST CANCER GENE” in a diseased cell. The values can be mRNA expression levels obtained from experiments, e.g., microarray analysis. The values can also be mRNA levels normalised relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more “BREAST CANCER GENES” in a first cell, e.g., a cell of a subject, and that in a second cell, comprising obtaining the level of expression of one or more “BREAST CANCER GENES” in a first cell and entering these values into a computer comprising a database including records comprising values corresponding to levels of expression of one or more “BREAST CANCER GENES” in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of “BREAST CANCER GENES” are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises values of expression levels in cells of subjects at different stages of breast cancer, and the computer is capable of comparing expression data entered into the computer with the data stored, and produce results indicating to which of the expression profiles in the computer, the one entered is most similar, such as to determine the stage of breast cancer in the subject.

In yet another embodiment, the reference expression profiles in the computer are expression profiles from cells of breast cancer of one or more subjects, which cells are treated in vivo or in vitro with a drug used for therapy of breast cancer. Upon entering of expression data of a cell of a subject treated in vitro or in vivo with the drug, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of breast cancer in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of breast cancer. The reference cells can also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides a method for selecting a therapy for a patient having breast cancer, the method comprising: (i) providing the level of expression of one or more genes characteristic of breast cancer in a diseased cell of the patient; (ii) providing a plurality of reference profiles, each associated with a therapy, wherein the subject expression profile and each reference profile has a plurality of values, each value representing the level of expression of a gene characteristic of breast cancer; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of an mRNA in two biological samples can be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expression comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of a stage of breast cancer or to a treatment for breast cancer.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputting device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes characteristic of breast cancer. The database may contain one or more expression profiles of genes characteristic of breast cancer in different cells.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic gene set database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

### Detection of Variant Polynucleotide Sequence

In yet another embodiment, the invention provides methods for determining whether a subject is at risk for developing a disease, such as a predisposition to develop malignant neoplasia, for example breast cancer, associated with an aberrant activity of any one of the polypeptides encoded by any of the polynucleotides of the SEQ ID NO: 1 to 26 or 53 to 75, wherein the aberrant activity of the polypeptide is characterized by detecting the presence or absence of a genetic lesion characterized by at least one of these:
(i) an alteration affecting the integrity of a gene encoding a marker polypeptides, or
(ii) the misexpression of the encoding polynucleotide.

To illustrate, such genetic lesions can be detected by ascertaining the existence of at least one of these:
I. a deletion of one or more nucleotides from the polynucleotide sequence
II. an addition of one or more nucleotides to the polynucleotide sequence
III. a substitution of one or more nucleotides of the polynucleotide sequence
IV. a gross chromosomal rearrangement of the polynucleotide sequence
V. a gross alteration in the level of a messenger RNA transcript of the polynucleotide sequence
VI. aberrant modification of the polynucleotide sequence, such as of the methylation pattern of the genomic DNA
VII. the presence of a non-wild type splicing pattern of a messenger RNA transcript of the gene
VIII. a non-wild type level of the marker polypeptide
IX. allelic loss of the gene
X. allelic gain of the gene
XI. inappropriate post-translational modification of the marker polypeptide

The present Invention provides assay techniques for detecting mutations in the encoding polynucleotide sequence. These methods include, but are not limited to, methods involving sequence analysis, Southern blot hybridization, restriction enzyme site mapping, and methods involving detection of absence of nucleotide pairing between the polynucleotide to be analyzed and a probe.

Specific diseases or disorders, e.g., genetic diseases or disorders, are associated with specific allelic variants of polymorphic regions of certain genes, which do not necessarily encode a mutated protein. Thus, the presence of a specific allelic variant of a polymorphic region of a gene in a subject can render the subject susceptible to developing a specific disease or disorder. Polymorphic regions in genes, can be identified, by determining the nucleotide sequence of genes in populations of individuals. If a polymorphic region is identified, then the link with a specific disease can be determined by studying specific populations of individuals, e.g. individuals which developed a specific disease, such as breast cancer. A polymorphic region can be located in any region of a gene, e.g., exons, in coding or non coding regions of exons, introns, and promoter region.

In an exemplary embodiment, there is provided a polynucleotide composition comprising a polynucleotide probe including a region of nucleotide sequence which is capable of hybridising to a sense or antisense sequence of a gene or naturally occurring mutants thereof, or 5′ or 3′ flanking sequences or intronic sequences naturally associated with the subject genes or naturally occurring mutants thereof. The polynucleotide of a cell is rendered accessible for hybridization, the probe is contacted with the polynucleotide of the sample, and the hybridization of the probe to the sample polynucleotide is detected. Such techniques can be used to detect lesions or allelic variants at either the genomic or mRNA level, including deletions, substitutions, etc., as well as to determine mRNA transcript levels.

A preferred detection method is allele specific hybridization using probes overlapping the mutation or polymorphic site and having about 5, 10, 20, 25, or 30 nucleotides around the mutation or polymorphic region. In a preferred embodiment of the invention, several probes capable of hybridising specifically to allelic variants are attached to a solid phase support, e.g., a “chip”. Mutation detection analysis using these chips comprising oligonucleotides, also termed “DNA probe arrays” is described e.g., in Cronin et al. (119). In one embodiment, a chip comprises all the allelic variants of at least one polymorphic region of a gene. The solid phase support is then contacted with a test polynucleotide and hybridization to the specific probes is detected. Accordingly, the identity of numerous allelic variants of one or more genes can be identified in a simple hybridization experiment.

In certain embodiments, detection of the lesion comprises utilizing the probe/primer in a polymerase chain reaction (PCR) (see, e.g. U.S. Pat. Nos. 4,683,195 and 4,683,202) such as anchor PCR or RACE PCR, or, alternatively, in a ligase chain reaction (LCR) [Landegran et al., 1988, (120) and Nakazawa et al., 1994 (121)], the latter of which can be particularly useful for detecting point mutations in the gene; Abravaya et al., 1995, (122)]. In a merely illustrative embodiment, the method includes the steps of (i) collecting a sample of cells from a patient, (ii) isolating polynucleotide (e.g., genomic, mRNA or both) from the cells of the sample, (iii) contacting the polynucleotide sample with one or more primers which specifically hybridize to a polynucleotide sequence under conditions such that hybridization and amplification of the polynucleotide (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication [Guatelli, J. C. et al., 1990, (123)], transcriptional amplification system [Kwoh, D. Y. et al., 1989, (124)], Q-Beta replicase [Lizardi, P. M. et al., 1988, (125)], or any other polynucleotide amplification method, followed by the detection of the amplified molecules using techniques well known to those of skill in the art. These detection schemes are especially useful for the detection of polynucleotide molecules if such molecules are present in very low numbers.

In a preferred embodiment of the subject assay, mutations in, or allelic variants, of a gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover; the use of sequence specific ribozymes (see, for example, U.S. Pat. No. 5,498,531) can be used to score for the presence of specific mutations by development or loss of a ribozyme cleavage site.

### In Situ Hybridization

In one aspect, the method comprises in situ hybridization with a probe derived from a given marker polynucleotide, which sequence is selected from any of the polynucleotide sequences of the SEQ ID NO: 1 to 9, or 11 to 19 or 21 to 26 and 53 to 75 or a sequence complementary thereto. The method comprises contacting the labeled hybridization probe with a sample of a given type of tissue from a patient potentially having malignant neoplasia and breast cancer in particular as well as normal tissue from a person with no malignant neoplasia, and determining whether the probe labels tissue of the patient to a degree significantly different (e.g., by at least a factor of two, or at least a factor of five, or at least a factor of twenty, or at least a factor of fifty) than the degree to which normal tissue is labelled.

Polypeptide Detection

The subject invention further provides a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

### Antibodies

Any type of antibody known in the art can be generated to bind specifically to an epitope of a “BREAST CANCER GENE” polypeptide. An antibody as used herein includes intact immuno-globulin molecules, as well as fragments thereof, such as Fab, F(ab)₂, and Fv, which are capable of binding an epitope of a “BREAST CANCER GENE” polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a “BREAST CANCER GENE” polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a “BREAST CANCER GENE” polypeptide provides a detection signal at least 5-, 10-, or 20-fold higher than a detection signal provided with other proteins when used in an immunochemical assay. Preferably, antibodies which specifically bind to “BREAST CANCER GENE” polypeptides do not detect other proteins in immunochemical assays and can immunoprecipitate a “BREAST CANCER GENE” polypeptide from solution.

“BREAST CANCER GENE” polypeptides can be used to immunize a mammal, such as a mouse, rat, rabbit, guinea pig, monkey, or human, to produce polyclonal antibodies. If desired, a “BREAST CANCER GENE” polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a “BREAST CANCER GENE” polypeptide can be prepared using any technique which provides for the production of antibody molecules by continuous cell lines in culture. These techniques include, but are not limited to, the hybridoma technique, the human B cell hybridoma technique, and the EBV hybridoma technique [Kohler et al., 1985, (136); Kozbor et al., 1985, (137); Cote et al., 1983, (138) and Cole et al., 1984, (139)].

In addition, techniques developed for the production of chimeric antibodies, the splicing of mouse antibody genes to human antibody genes to obtain a molecule with appropriate antigen specificity and biological activity, can be used [Morrison et al., 1984, (140); Neuberger et al., 1984, (141); Takeda et al., 1985, (142)]. Monoclonal and other antibodies also can be humanized to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a “BREAST CANCER GENE” polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to “BREAST CANCER GENE” polypeptides. Antibodies with related specificity, but of distinct idiotypic composition, can be generated by chain shuffling from random combinatorial immunoglobulin libraries [Burton, 1991, (143)].

Single-chain antibodies also can be constructed using a DNA amplification method, such as PCR, using hybridoma cDNA as a template [Thirion et al., 1996, (144)]. Single-chain antibodies can be mono- or bispecific, and can be bivalent or tetravalent Construction of tetravalent, bispecific single-chain antibodies is taught, for example, in Coloma & Morrison, (145). Construction of bivalent, bispecific single-chain antibodies is taught in Mallender & Voss, (146).

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology [Verhaar et al., 1995, (147); Nicholls et al., 1993, (148)].

Antibodies which specifically bind to “BREAST CANCER GENE” polypeptides also can be produced by inducing in vivo production in the lymphocyte population or by screening immunoglobulin libraries or panels of highly specific binding reagents as disclosed in the literature [Orlandi et al., 1989, (149) and Winter et al., 1991, (150)].

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the antibodies described in WO 94/13804, also can be prepared.

Antibodies according to the invention can be purified by methods well known in the art. For example, antibodies can be affinity purified by passage over a column to which a “BREAST CANCER GENE” polypeptide is bound. The bound antibodies can then be eluted from the column using a buffer with a high salt concentration.

Immunoassays are commonly used to quantify the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarisation immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art

In another embodiment, the level of at least one product encoded by any of the polynucleotide sequences of the SEQ ID NO: 2 to 6, 8, 9, 11 to 16, 18, 19 or 21 to 26 or 53 to 75 or of at least 2 products encoded by a polynucleotide selected from SEQ ID NO: 1 to 26 and 53 to 75 or a sequence complementary thereto, in a biological fluid (e.g., blood or urine) of a patient may be determined as a way of monitoring the level of expression of the marker polynucleotide sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of the disorder, e.g., plaque formation. The level of the marker polypeptide can be used predictively to evaluate whether a sample of cells contains cells which are, or are predisposed towards becoming, plaque associated cells. The observation of marker polypeptide level can be utilized in decisions regarding, e.g., the use of more stringent therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantify the level of marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

### Immunohistochemistry

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissues samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a Label for subsequent detection of binding. samples are incubated for a time Sufficient for formation of the immunocomplexes. Binding of the antibody is then detected by virtue of a Label conjugated to this antibody. Where the antibody is unlabelled, a second labeled antibody may be employed, e.g., which is specific for the isotype of the anti-marker polypeptide antibody. Examples of labels which may be employed include radionuclides, fluorescence, chemiluminescence, and enzymes.

Where enzymes are employed, the Substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination of the average amount of the marker polypeptide associated with a Single cell by correlating the amount of marker polypeptide in a cell-free extract produced from a predetermined number of cells.

In yet another embodiment, the invention contemplates using one or more antibodies which are generated against one or more of the marker polypeptides of this invention, which polypeptides are encoded by any of the polynucleotide sequences of the SEQ ID NO: 1 to 26 or 53 to 75. Such a panel of antibodies may be used as a reliable diagnostic probe for breast cancer. The assay of the present invention comprises contacting a biopsy sample containing cells, e.g., macrophages, with a panel of antibodies to one or more of the encoded products to determine the presence or absence of the marker polypeptides.

The diagnostic methods of the subject invention may also be employed as follow-up to treatment e.g., quantification of the level of marker polypeptides may be indicative of the effectiveness of current or previously employed therapies for malignant neoplasia and breast cancer in particular as well as the effect of these therapies upon patient prognosis.

The diagnostic assays described above can be adapted to be used as prognostic assays, as well. Such an application takes advantage of the sensitivity of the assays of the Invention to events which take place at characteristic stages in the progression of plaque generation in case of malignant neoplasia. For example, a given marker gene may be up- or down-regulated at a very early stage, perhaps before the cell is developing into a foam cell, while another marker gene may be characteristically up or down regulated only at a much later stage. Such a method could involve the steps of contacting the mRNA of a test cell with a polynucleotide probe derived from a given marker polynucleotide which is expressed at different characteristic levels in breast cancer tissue cells at different stages of malignant neoplasia progression, and determining the approximate amount of hybridization of the probe to the mRNA of the cell, such amount being an indication of the level of expression of the gene in the cell, and thus an indication of the stage of disease progression of the cell; alternatively, the assay can be carried out with an antibody specific for the gene product of the given marker polynucleotide, contacted with the proteins of the test cell. A battery of such tests will disclose not only the existence of a certain arteriosclerotic plaque, but also will allow the clinician to select the mode of treatment most appropriate for the disease, and to predict the likelihood of success of that treatment.

The methods of the invention can also be used to follow the clinical course of a given breast cancer predisposition. For example, the assay of the Invention can be applied to a blood sample from a patient; following treatment of the patient for BREAST CANCER, another blood sample is taken and the test repeated. Successful treatment will result in removal of demonstrate differential expression, characteristic of the breast cancer tissue cells, perhaps approaching or even surpassing normal levels.

### Polypeptide Activity

In one embodiment the present invention provides a method for screening potentially therapeutic agents which modulate the activity of one or more “BREAST CANCER GENE” polypeptides, such that if the activity of the polypeptide is increased as a result of the upregulation of the “BREAST CANCER GENE” in a subject having or at risk for malignant neoplasia and breast cancer in particular, the therapeutic substance will decrease the activity of the polypeptide relative to the activity of the some polypeptide in a subject not having or not at risk for malignant neoplasia or breast cancer in particular but not treated with the therapeutic agent. Likewise, if the activity of the polypeptide as a result of the downregulation of the “BREAST CANCER GENE” is decreased in a subject having or at risk for malignant neoplasia or breast cancer in particular, the therapeutic agent will increase the activity of the polypeptide relative to the activity of the same polypeptide in a subject not having or not at risk for malignant neoplasia or breast cancer in particular, but not treated with the therapeutic agent.

The activity of the “BREAST CANCER GENE” polypeptides indicated in Table 2 or 3 may be measured by any means known to those of skill in the art, and which are particular for the type of activity performed by the particular polypeptide. Examples of specific assays which may be used to measure the activity of particular polynucleotides are shown below.

a) G protein Coupled Receptors

In one embodiment, the “BREAST CANCER GENE” polynucleotide may encode a G protein coupled receptor. In one embodiment, the present invention provides a method of screening potential modulators (inhibitors or activators) of the G protein coupled receptor by measuring changes in the activity of the receptor in the presence of a candidate modulator.

### 1. Gᵢ-Coupled Receptors

Cells (such as CHO cells or primary cells) are stably transfected with the relevant receptor and with an inducible CRE-luciferase construct. Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 2000 cells/well in 35 μl cell culture medium) in DMEM/F12 with FBS, and are grown for 48 hours (range: ˜24-60 hours, depending on cell line). Growth medium is then exchanged against serum free medium (SFM; e.g. Ultra-CHO), containing 0.1% BSA. Test compounds dissolved in DMSO are diluted in SFM and transferred to the test cultures (maximal final concentration 10 μmolar), followed by addition of forskolin (˜1 μmolar, final conc.) in SFM+0.1% BSA 10 minutes later. In case of antagonist screening both, an appropriate concentration of agonist, and forskolin are added. The plates are incubated at 37° C. in 10% CO₂ for 3 hours. Then the supernatant is removed, cells are lysed with lysis reagent (25 mmolar phosphate-buffer, pH 7,8, containing 2 mmolar DDT, 10% glycerol and 3% Triton X100). The luciferase reaction is started by addition of substrate-buffer (e.g. luciferase assay reagent, Promega) and luminescence is immediately determined (e.g. Berthold luminometer or Hamamatzu camera system).

### 2. Gₛ-coupled Receptors

Cells (such as CHO cells or primary cells) are stably transfected with the relevant receptor and with an inducible CRE-luciferase construct Cells are grown in 50% Dulbecco's modified Eagle medium/50% F12 (DMEM/F12) supplemented with 10% FBS, at 37° C. in a humidified atmosphere with 10% CO₂ and are routinely split at a ratio of 1:10 every 2 or 3 days. Test cultures are seeded into 384-well plates at an appropriate density (e.g. 1000 or 2000 cells/well in 35 μl cell culture medium) in DMEM/F12 with FBS, and are grown for 48 hours (range: ˜24-60 hours, depending on cell line). The assay is started by addition of test-compounds in serum free medium (SFM; e.g. Ultra-CHO) containing 0.1% BSA: Test compounds are dissolved in DMSO, diluted in SFM and transferred to the test cultures (maximal final concentration 10 μmolar, DMSO conc. <0.6%). In case of antagonist screening an appropriate concentration of agonist is added 5-10 minutes later. The plates are incubated at 37° C. in 10% CO₂ for 3 hours. Then the cells are lysed with 10 μl lysis reagent per well (25 mmolar phosphate-buffer, pH 7,8, containing 2 mmolar DDT, 10% glycerol and 3% Triton X100) and the luciferase reaction is started by addition of 20 μl substrate-buffer per well (e.g. luciferase assay reagent, Promega). Measurement of luminescence is started immediately (e.g. Berthold luminometer or Hamamatzu camera system).

### 3. G_{g}-coupled Receptors

Cells (such as CHO cells or primary cells) are stably transfected with the relevant receptor. Cells expressing functional receptor protein are grown in 50% Dulbecco's modified Eagle medium/50% F12 (DMEM/F12) supplemented with 10% FBS, at 37° C. in a humidified atmosphere with 5% CO₂ and are routinely split at a cell line dependent ratio every 3 or 4 days. Test cultures are seeded into 384-well plates at an appropriate density (e.g. 2000 cells/well in 35 μl cell culture medium) in DMEM/F12 with FBS, and are grown for 48 hours (range: ˜24-60 hours, depending on cell line). Growth medium is then exchanged against physiological salt solution (e.g. Tyrode solution). Test compounds dissolved in DMSO are diluted in Tyrode solution containing 0.1% BSA and transferred to the test cultures (maximal final concentration 10 μmolar). After addition of the receptor specific agonist the resulting Gq-mediated intracellular calcium increase is measured using appropriate read-out systems (e.g. calcium-sensitive dyes).

b) Ion Channels

Ion channels are integral membrane proteins involved in electrical signaling, transmembrane signal transduction, and electrolyte and solute transport. By forming macromolecular pores through the membrane lipid bilayer, ion channels account for the flow of specific ion species driven by the electrochemical potential gradient for the permeating ion. At the single molecule level, individual channels undergo conformational transitions (“gating”) between the ‘open’ (ion conducting) and ‘closed’ (non conducting) state. Typical single channel openings last for a few milliseconds and result in elementary transmembrane currents in the range of 10⁻⁹-10⁻¹² Ampere. Channel gating is controlled by various chemical and/or biophysical parameters, such as neurotransmitters and intracellular second messengers (‘ligand-gated’ channels) or membrane potential (‘voltage-gated’ channels). Ion channels are functionally characterized by their ion selectivity, gating properties, and regulation by hormones and pharmacological agents. Because of their central role in signaling and transport processes, ion channels present ideal targets for pharmacological therapeutics in various pathophysiological settings.

In one embodiment, the “BREAST CANCER GENE” may encode an ion channel. In one embodiment, the present invention provides a method of screening potential activators or inhibitors of channels activity of the “BREAST CANCER GENE” polypeptide. Screening for compounds interaction with ion channels to either inhibit or promote their activity can be based on (1.) binding and (2.) functional assays in living cells [Hille (183)].
1. For ligand-gated channels, e.g. ionotropic neurotransmitter/hormone receptors, assays can be designed detecting binding to the target by competition between the compound and a labeled ligand.
2. Ion channel function can be tested functionally in living cells. Target proteins are either expressed endogenously in appropriate reporter cells or are introduced recombinantly. Channel activity can be monitored by (2.1) concentration changes of the permeating ion (most prominently Ca²⁺ ions), (2.2) by changes in the transmembrane electrical potential gradient, and (2.3) by measuring a cellular response (e.g. expression of a reporter gene, secretion of a neurotransmitter) triggered or modulated by the target activity.
2.1 Channel activity results in transmembrane ion fluxes. Thus activation of ionic channels can be monitored by the resulting changes in intracellular ion concentrations using luminescent or fluorescent indicators. Because of its wide dynamic range and availability of suitable indicators this applies particularly to changes in intracellular Ca²⁺ ion concentration ([Ca²⁺]ᵢ). [Ca²⁺]ᵢ can be measured, for example, by aequorin luminescence or fluorescence dye technology (e.g. using Fluo-3, Indo-1, Fura-2). Cellular assays can be designed where either the Ca²⁺ flux through the target channel itself is measured directly or where modulation of the target channel affects membrane potential and thereby the activity of co-expressed voltage-gated Ca²⁺ channels.
2.2 Ion channel currents result in changes of electrical membrane potential (Vₘ) which can be monitored directly using potentiometric fluorescent probes. These electrically charged indicators (e.g. the anionic oxonol dye DiBAC₄(3)) redistribute between extra- and intracellular compartment in response to voltage changes. The equilibrium distribution is governed by the Nemst-equation. Thus changes in membrane potential results in concomitant changes in cellular fluorescence. Again, changes in Vₘ might be caused directly by the activity of the target ion channel or through amplification and/or prolongation of the signal by channels co-expressed in the same cell.
2.3 Target channel activity can cause cellular Ca²⁺ entry either directly or through activation of additional Ca²⁺ channel (see 2.1). The resulting intracellular Ca²⁺ signals regulate a variety of cellular responses, e.g. secretion or gene transcription. Therefore modulation of the target channel can be detected by monitoring secretion of a known hormone/transmitter from the target-expressing cell or through expression of a reporter gene (e.g. luciferase) controlled by an Ca²⁺-responsive promoter element (e.g. cyclic AMP/Ca²⁺-responsive elements; CRE).
   c) DNA-Binding Proteins and Transcription Factors

In one embodiment, the “BREAST CANCER GENE” may encode a DNA-binding protein or a transcription factor. The activity of such a DNA-binding protein or a transcription factor may be measured, for example, by a promoter assay which measures the ability of the DNA-binding protein or the transcription factor to initiate transcription of a test sequence linked to a particular promoter. In one embodiment, the present invention provides a method of screening test compounds for its ability to modulate the activity of such a DNA-binding protein or a transcription factor by measuring the changes in the expression of a test gene which is regulated by a promoter which is responsive to the transcription factor.

d) Promotor Assays

A promoter assay was set up with a human hepatocellular carcinoma cell HepG2 that was stably transfected with a luciferase gene under the control of a gene of interest (e.g. thyroid hormone) regulated promoter. The vector 2xIROluc, which was used for transfection, carries a thyroid hormone responsive element (TRE) of two 12 bp inverted palindromes separated by an 8 bp spacer in front of a tk minimal promoter and the luciferase gene. Test cultures were seeded in 96 well plates in serum-free Eagle's Minimal Essential Medium supplemented with glutamine, tricine, sodium pyruvate, non-essential amino acids, insulin, selen, transferrin, and were cultivated in a humidified atmosphere at 10% CO₂ at 37° C. After 48 hours of incubation serial dilutions of test compounds or reference compounds (L-T3, L-T4 e.g.) and co-stimulator if appropriate (final concentration 1 nM) were added to the cell cultures and incubation was continued for the optimal time (e.g. another 4-72 hours). The cells were then lysed by addition of buffer containing Triton X100 and luciferin and the luminescence of luciferase induced by T3 or other compounds was measured in a luminometer. For each concentration of a test compound replicates of 4 were tested. EC₅₀-values for each test compound were calculated by use of the Graph Pad Prism Scientific software.

### Screening Methods

The invention provides assays for screening test compounds which bind to or modulate the activity of a “BREAST CANCER GENE” polypeptide or a “BREAST CANCER GENE” polynucleotide. A test compound preferably binds to a “BREAST CANCER GENE” polypeptide or polynucleotide. More preferably, a test compound decreases or increases “BREAST CANCER GENE” activity by at least about 10, preferably about 50, more preferably about 75, 90, or 100% relative to the absence of the test compound.

### Test Compounds

Test compounds can be pharmacological agents already known in the art or can be compounds previously unknown to have any pharmacological activity. The compounds can be naturally occurring or designed in the laboratory. They can be isolated from microorganisms, animals, or plants, and can be produced recombinant, or synthesised by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the one-bead one-compound library method, and synthetic library methods using affinity chromatography selection. The biological library approach is limited to polypeptide libraries, while the other four approaches are applicable to polypeptide, non-peptide oligomer, or small molecule libraries of compounds. [For review see Lam, 1997, (151)].

Methods for the synthesis of molecular libraries are well known in the art [see, for example, DeWitt et al., 1993, (152); Erb et al., 1994, (153); Zuckermann et al., 1994, (154); Cho et al., 1993, (155); Carell et al., 1994, (156) and Gallop et al., 1994, (157). Libraries of compounds can be presented in solution [see, e.g., Houghten, 1992, (158)], or on beads [Lam, 1991, (159)], DNA-chips [Fodor, 1993, (160)], bacteria or spores (Ladner, U.S. Pat. No. 5,223,409), plasmids [Cull et al., 1992, (161)], or phage [Scott & Smith, 1990, (162); Devlin, 1990, (163); Cwirla et al., 1990, (164); Felici, 1991, (165)].

### High Throughput Screening

Test compounds can be screened for the ability to bind to “BREAST CANCER GENE” polypeptides or polynucleotides or to affect “BREAST CANCER GENE” activity or “BREAST CANCER GENE” expression using high throughput screening. Using high throughput screening, many discrete compounds can be tested in parallel so that large numbers of test compounds can be quickly screened. The most widely established techniques utilize 96-well, 384-well or 1536-well microtiter plates. The wells of the microtiter plates typically require assay volumes that range from 5 to 500 μl. In addition to the plates, many instruments, materials, pipettors, robotics, plate washers, and plate readers are commercially available to fit the microwell formats.

Alternatively, free format assays, or assays that have no physical barrier between samples, can be used. For example, an assay using pigment cells (melanocytes) in a simple homogeneous assay for combinatorial peptide libraries is described by Jayawickreme et al., (166). The cells are placed under agarose in culture dishes, then beads that carry combinatorial compounds are placed on the surface of the agarose. The combinatorial compounds are partially released the compounds from the beads. Active compounds can be visualised as dark pigment areas because, as the compounds diffuse locally into the gel matrix, the active compounds cause the cells to change colors.

Another example of a free format assay is described by Chelsky, (167). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV light. Compounds that inhibited the enzyme were observed as local zones of inhibition having less color change.

In another example, combinatorial libraries were screened for compounds that had cytotoxic effects on cancer cells growing in agar [Salmon et al., 1996, (168)].

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

### Binding Assays

For binding assays, the test compound is preferably a small molecule which binds to and occupies, for example, the ATP/GTP binding site of the enzyme or the active site of a “BREAST CANCER GENE” polypeptide, such that normal biological activity is prevented. Examples of such small molecules include, but are not limited to, small peptides or peptide-like molecules.

In binding assays, either the test compound or a “BREAST CANCER GENE” polypeptide can comprise a detectable label, such as a fluorescent, radioisotopic, chemiluminescent, or enzymatic label, such as horseradish peroxidase, alkaline phosphatase, or luciferase. Detection of a test compound which is bound to a “BREAST CANCER GENE” polypeptide can then be accomplished, for example, by direct counting of radioemmission, by scintillation counting, or by determining conversion of an appropriate substrate to a detectable product.

Alternatively, binding of a test compound to a “BREAST CANCER GENE” polypeptide can be determined without labeling either of the interactants. For example, a microphysiometer can be used to detect binding of a test compound with a “BREAST CANCER GENE” polypeptide. A microphysiometer (e.g., CytosensorJ) is an analytical instrument that measures the rate at which a cell acidifies its environment using a light-addressable potentiometric sensor (LAPS). Changes in this acidification rate can be used as an indicator of the interaction between a test compound and a “BREAST CANCER GENE” polypeptide [McConnell et al., 1992, (169)].

Determining the ability of a test compound to bind to a “BREAST CANCER GENE” polypeptide also can be accomplished using a technology such as real-time Bimolecular Interaction Analysis (BIA) [Sjolander & Urbaniczky, 1991, (170), and Szabo et al., 1995, (171)]. BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a “BREAST CANCER GENE” polypeptide can be used as a “bait protein” in a two-hybrid assay or three-hybrid assay [see, e.g., U.S. Pat. No. 5,283,317; Zervos et al., 1993, (172); Madura et al., 1993, (173); Bartel et al., 1993, (174); Iwabuchi et al., 1993, (175) and Brent WO 94/10300], to identify other proteins which bind to or interact with the “BREAST CANCER GENE” polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a “BREAST CANCER GENE” polypeptide can be fused to a polynucleotide encoding the DNA binding domain of a known transcription factor (e.g., GAL4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the “BREAST CANCER GENE” polypeptide.

It may be desirable to immobilize either a “BREAST CANCER GENE” polypeptide (or polynucleotide) or the test compound to facilitate separation of bound from unbound forms of one or both of the interactants, as well as to accommodate automation of the assay. Thus, either a “BREAST CANCER GENE” polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach a “BREAST CANCER GENE” polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a “BREAST CANCER GENE” polypeptide (or polynucleotide) can be accomplished in any vessel suitable for containing the reactants. Examples of such vessels include microtiter plates, test tubes, and microcentrifuge tubes.

In one embodiment, a “BREAST CANCER GENE” polypeptide is a fusion protein comprising a domain that allows the “BREAST CANCER GENE” polypeptide to be bound to a solid support. For example, glutathione S-transferase fusion proteins can be adsorbed onto glutathione sepharose beads (Sigma Chemical, St Louis, Mo.) or glutathione derivatized microtiter plates, which are then combined with the test compound or the test compound and the nonadsorbed “BREAST CANCER GENE” polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilising proteins or polynucleotides on a solid support also can be used in the screening assays of the invention. For example, either a “BREAST CANCER GENE” polypeptide (or polynucleotide) or a test compound can be immobilized utilizing conjugation of biotin and streptavidin. Biotinylated “BREAST CANCER GENE” polypeptides (or polynucleotides) or test compounds can be prepared from biotin NHS (N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a “BREAST CANCER GENE” polypeptide, polynucleotide, or a test compound, but which do not interfere with a desired binding site, such as the ATP/GTP binding site or the active site of the “BREAST CANCER GENE” polypeptide, can be derivatised to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to a “BREAST CANCER GENE” polypeptide or test compound, enzyme-linked assays which rely on detecting an activity of a “BREAST CANCER GENE” polypeptide, and SDS gel electrophoresis under non-reducing conditions.

Screening for test compounds which bind to a “BREAST CANCER GENE” polypeptide or polynucleotide also can be carried out in an intact cell. Any cell which comprises a “BREAST CANCER GENE” polypeptide or polynucleotide can be used in a cell-based assay system. A “BREAST CANCER GENE” polynucleotide can be naturally occurring in the cell or can be introduced using techniques such as those described above. Binding of the test compound to a “BREAST CANCER GENE” polypeptide or polynucleotide is determined as described above.

### Modulation of Gene Expression

In another embodiment, test compounds which increase or decrease “BREAST CANCER GENE” expression are identified. A “BREAST CANCER GENE” polynucleotide is contacted with a test compound, and the expression of an RNA or polypeptide product of the “BREAST CANCER GENE” polynucleotide is determined. The level of expression of appropriate mRNA or poly-peptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of “BREAST CANCER GENE” mRNA or polypeptide expression in the cells can be determined by methods well known in the art for detecting mRNA or polypeptide. Either qualitative or quantitative methods can be used. The presence of polypeptide products of a “BREAST CANCER GENE” polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a “BREAST CANCER GENE” polypeptide.

Such screening can be carried out either in a cell-free assay system or in an intact cell. Any cell which expresses a “BREAST CANCER GENE” polynucleotide can be used in a cell-based assay system. A “BREAST CANCER GENE” polynucleotide can be naturally occurring in the cell or can be introduced using techniques such as those described above. Either a primary culture or an established cell line, such as CHO or human embryonic kidney 293 cells, can be used.

### Therapeutic Indications and Methods

Therapies for treatment of breast cancer primarily relied upon effective chemotherapeutic drugs for intervention on the cell proliferation, cell growth or angiogenesis. The advent of genomics-driven molecular target identification has opened up the possibility of identifying new breast cancer-specific targets for therapeutic intervention that will provide safer, more effective treatments for malignant neoplasia patients and breast cancer patients in particular. Thus, newly discovered breast cancer-associated genes and their products can be used as tools to develop innovative therapies. The identification of the Her2/neu receptor kinase presents exciting new opportunities for treatment of a certain subset of tumor patients as described before. Genes playing important roles in any of the physiological processes outlined above can be characterized as breast cancer targets. Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Modulators of target gene expression or protein activity can be identified in this manner and subsequently tested in cellular and in vivo disease models for therapeutic activity. Optimization of lead compounds with iterative testing in biological models and detailed pharmacokinetic and toxicological analyses form the basis for drug development and subsequent testing in humans.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense polynucleotide molecule, a specific antibody, ribozyme, or a human “BREAST CANCER GENE” polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above described screening assays for treatments as described herein.

A reagent which affects human “BREAST CANCER GENE” activity can be administered to a human cell, either in vitro or in vivo, to reduce or increase human “BREAST CANCER GENE” activity. The reagent preferably binds to an expression product of a human “BREAST CANCER GENE”. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmol of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes usually used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al., 1993, (176); Chiou et al., 1994, (177); Wu & Wu, 1988, (178); Wu et al., 1994, (179); Zenke et al., 1990, (180); Wu et al., 1991, (181).

### Determination of a Therapeutically Effective Dose

The determination of a therapeutically effective dose is well within the capability of those skilled in the art A therapeutically effective dose refers to that amount of active ingredient which increases or decreases human “BREAST CANCER GENE” activity relative to the human “BREAST CANCER GENE” activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, a gene gun, and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a “BREAST CANCER GENE” gene or the activity of a “BREAST CANCER GENE” polypeptide by at least about 10, preferably about 50, more preferably about 75, 90, or 100% relative to the absence of the reagent. The effectiveness of the mechanism chosen to decrease the level of expression of a “BREAST CANCER GENE” gene or the activity of a “BREAST CANCER GENE” polypeptide can be assessed using methods well known in the art, such as hybridization of nucleotide probes to “BREAST CANCER GENE”-specific mRNA, quantitative RT-PCR, immunologic detection of a “BREAST CANCER GENE” polypeptide, or measurement of “BREAST CANCER GENE” activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, birds and mammals such as dogs, cats, cows, pigs, sheep, goats, horses, rabbits, monkeys, and most preferably, humans.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### Pharmaceutical Compositions

The invention also provides pharmaceutical compositions which can be administered to a patient to achieve a therapeutic effect. Pharmaceutical compositions of the invention can comprise, for example, a “BREAST CANCER GENE” polypeptide, “BREAST CANCER GENE” polynucleotide, ribozymes or antisense oligonucleotides, antibodies which specifically bind to a “BREAST CANCER GENE” polypeptide, or mimetics, agonists, antagonists, or inhibitors of a “BREAST CANCER GENE” polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intraarterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 150 mM histidine, 0.1%2% sucrose, and 27% mannitol, at a pH range of 4.5 to 5.5, that is combined with buffer prior to use.

Further details on techniques for formulation and administration can be found in the latest edition of REMINGTON'S PHARMACEUTICAL SCIENCES (182). After pharmaceutical compositions have been prepared, they can be placed in an appropriate container and labeled for treatment of an indicated condition. Such labeling would include amount, frequency, and method of administration.

### Material and Methods

One strategy for identifying genes that are involved in breast cancer is to detect genes that are expressed differentially under conditions associated with the disease versus non-disease conditions. The sub-sections below describe a number of experimental systems which may be used to detect such differentially expressed genes. In general, these experimental systems include at least one experimental condition in which subjects or samples are treated in a manner associated with breast cancer, in addition to at least one experimental control condition lacking such disease associated treatment. Differentially expressed genes are detected, as described below, by comparing the pattern of gene expression between the experimental and control conditions.

Once a particular gene has been identified through the use of one such experiment, its expression pattern may be further characterized by studying its expression in a different experiment and the findings may be validated by an independent technique. Such use of multiple experiments may be useful in distinguishing the roles and relative importance of particular genes in breast cancer. A combined approach, comparing gene expression pattern in cells derived from breast cancer patients to those of in vitro cell culture models can give substantial hints on the pathways involved in development and/or progression of breast cancer.

Among the experiments which may be utilized for the identification of differentially expressed genes involved in malignant neoplasia and breast cancer, for example, are experiments designed to analyze those genes which are involved in signal transduction. Such experiments may serve to identify genes involved in the proliferation of cells.

Below are methods described for the identification of genes which are involved in breast cancer. Such represent genes which are differentially expressed in breast cancer conditions relative to their expression in normal, or non-breast cancer conditions or upon experimental manipulation based on clinical observations. Such differentially expressed genes represent “target” and/or “marker” genes. Methods for the further characterization of such differentially expressed genes, and for their identification as target and/or marker genes, are presented below.

Alternatively, a differentially expressed gene may have its expression modulated, i.e., quantitatively increased or decreased, in normal versus breast cancer states, or under control versus experimental conditions. The degree to which expression differs in normal versus breast cancer or control versus experimental states need only be large enough to be visualized via standard characterization techniques, such as, for example, the differential display technique described below. Other such standard characterization techniques by which expression differences may be visualized include but are not limited to quantitative RT-PCR and Northern analyses, which are well known to those of skill in the art.

As part of this invention, a method is described by way of illustration and not by limitation, displaying at least some of the below mentioned aspects:
1. A method for the prediction, diagnosis or prognosis of malignant neoplasia by the detection of at least 2 markers characterized in that the markers are genes and fragments thereof or genomic nucleic acid sequences that are located on one chromosomal region which is altered in malignant neoplasia.
2. A method for the prediction, diagnosis or prognosis of malignant neoplasia by the detection of at least 2 markers characterized in that the markers are:
   a) genes that are located on one or more chromosomal region(s) which is/are altered in malignant neoplasia; and
   b)
      i) receptor and ligand; or
      ii) members of the same signal transduction pathway; or
      iii) members of synergistic signal transduction pathways; or
      iv) members of antagonistic signal transduction pathways; or
      v) transcription factor and transcription factor binding site.
3. The method of aspect 1 or 2 wherein the malignant neoplasia is breast cancer, ovarian cancer, gastric cancer, colon cancer, esophageal cancer, mesenchymal cancer, bladder cancer or non-small cell lung cancer.
4. The method of aspect 1 or 2 wherein at least one chromosomal region is defined as the cytogenetic region: 1p13, 1q32, 3p21-p24, 5p13-p14, 8q23-q24, 11q13, 12q13, 17q12-q24 or 20q13.
5. The method of aspect 1 or 2 wherein at least chromosomal region is defined as the cytogenetic region 17q11.2-21.3 and the malignant neoplasia is breast cancer, ovarian cancer, gastric cancer, colon cancer, esophageal cancer, mesenchymal cancer, bladder cancer or non-small cell lung cancer.
6. The method of aspect 1 or 2 wherein at least one chromosomal region is defined as the cytogenetic region 3p21-24 and the malignant neoplasia is breast cancer, ovarian cancer, gastric cancer, colon cancer, esophageal cancer, mesenchymal cancer, bladder cancer or non-small cell lung cancer.
7. The method of aspect 1 or 2 wherein at least one chromosomal region is defined as the cytogenetic region 12q13 and the malignant neoplasia is breast cancer, ovarian cancer, gastric cancer, colon cancer, esophageal cancer, mesenchymal cancer, bladder cancer or non-small cell lung cancer.
8. A method for the prediction, diagnosis or prognosis of malignant neoplasia by the detection of at least one marker whereby the marker is a VNTR, SNP, RFLP or STS characterized in that the marker is located on one chromosomal region which is altered in malignant neoplasia due to amplification and the marker is detected in a cancerous and a non-cancerous tissue or biological sample of the same individual.
9. The method of aspect 8 wherein the marker is selected from the group consisting of the VNTRS:
   D17S946, D17S1181, D17S2026, D17S838, D17S250, D17S1818, D17S614, D17S2019, D17S608, D17S1655, D17S2147, D17S754, D17S1814, D17S2007, D17S1246, D17S1979, D17S1984, D17S1984, D17S1867, D17S1788, D17S1836, D17S1787, D17S1660, D17S2154, D17S1955, D17S2098, D17S518, D17S1851, D11S4358, D17S964, D19S1091, D17S1179, D10S2160, D17S1230, D17S1338, D17S2011, D17S1237, D17S2038, D17S2091, D17S649, D17S1190 and M87506.
10. The method of aspect 8 wherein the marker is selected from the group consisting of the SNPs:
rs2230698, rs2230700, rs1058808, rs1801200, rs903506, rs2313170, rs1136201, rs2934968, rs2172826, rs1810132, rs1801201, rs2230702, rs2230701, rs1126503, rs3471, rs13695, rs471692, rs558068, rs1064288, rs1061692, rs520630, rs782774, rs565121, rs2586112, rs532299, rs2732786, rs1804539, rs1804538, rs1804537, rs1141364, rs12231, rs1132259, rs1132257, rs1132256, rs1132255, rs1132254, rs1132252, rs1132268 and rs1132258
11. A method for the prediction, diagnosis or prognosis of malignant neoplasia by the detection of at least one marker characterized in that the marker is selected from:
   a) a polynucleotide or polynucleotide analog comprising at least one of the sequences of SEQ ID NO: 2 to 6, 8, 9, 11 to 16, 18, 19, 21 to 26, 53 to 75, or 315 to 318;
   b) a polynucleotide or polynucleotide analog which hybridizes under stringent conditions to a polynucleotide specified in (a) and encodes a polypeptide exhibiting the same biological function as specified for the respective sequence in Table 2 or 3
   c) a polynucleotide or polynucleotide analog the sequence of which deviates from the polynucleotide specified in (a) and (c) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (d)
   e) a purified polypeptide encoded by a polynucleotide or polynucleotide analog sequence specified in (a) to (e)
   f) A purified polypeptide comprising at least one of the sequences of SEQ ID NO: 28 to 32, 34, 35, 37 to 42, 44, 45, 47 to 52, 76 to 98, or 393 to 396; are detected.
12. A method for the prediction, diagnosis or prognosis of malignant neoplasia by the detection of at least 2 markers characterized in that at least 2 markers are selected from:
   a) polynucleotide or polynucleotide analog comprising at least one of the sequences of SEQ ID NO: 1 to 26 or 53 to 75 or 315 to 318;
   b) a polynucleotide or polynucleotide analog which hybridizes under stringent conditions to a polynucleotide specified in (a) and encodes a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c)
   e) a purified polypeptide encoded by a polynucleotide sequence or polynucleotide analog specified in (a) to (d)
   f) a purified polypeptide comprising at least one of the sequences of SEQ ID NO: 27 to 52 or 76 to 98 or 393 to 396
   are detected.
13. The method of any of the aspects 1 or 12 wherein the detection method comprises the use of PCR, arrays or beads.
14. A diagnostic kit comprising instructions for conducting the method of any of aspects 1 to 13.
15. A composition for the prediction, diagnosis or prognosis of malignant neoplasia comprising:
   a) a detection agent for:
      i) any polynucleotide or polynucleotide analog comprising at least one of the sequences of SEQ ID NO: 2 to 6, 8, 9, 11 to 16, 18, 19, 21 to 26, 53 to 75, or 315 to 318,
      ii) any polynucleotide or polynucleotide analog which hybridizes under stringent conditions to a polynucleotide specified in (a) encoding a polypeptide exhibiting the same biological function as specified for the respective sequence in Table 2 or 3
      iii) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
      iv) a polynucleotide or polynucleotide analog which represents a specific fragment, derivative or allelic variation of a polynucleotide sequence specified in (a) to (c)
      v) a polypeptide encoded by a polynucleotide or polynucleotide analog sequence specified in (a) to (d);
      vi) a polypeptide comprising at least one of the sequences of SEQ ID NO: 28 to 32, 34, 35, 37 to 42, 44, 45, 47 to 52, 76 to 98, or 393 to 396.
   or
   b) at least 2 detection agents for at least 2 markers selected from:
      i) any polynucleotide comprising at least one of the sequences of SEQ ID NO: 1 to 26 or 53 to 75 or 315 to 318;
      ii) any polynucleotide which hybridizes under stringent conditions to a polynucleotide specified in (a) encoding a polypeptide exhibiting the same biological function as specified for the respective sequence in Table 2 or 3
      iii) a polynucleotide the sequence of which deviates from the polynucleotide specified in (a) and (b) due to the generation of the genetic code encoding a polypeptide exhibiting the same biological function as specified for the respective sequence in Table 2 or 3
      iv) a polynucleotide which represents a specific fragment, derivative or allelic variation of a polynucleotide sequence specified in (a) to (c)
      v) a polypeptide encoded by a polynucleotide sequence specified in (a) to (d);
      vi) a polypeptide comprising at least one of the sequences of SEQ ID NO: 27 to 52 or 76 to 98 or 393 to 396.
16. An array comprising a plurality of polynucleotides or polynucleotide analogs wherein each of the polynucleotides is selected from:
   a) a polynucleotide or polynucleotide analog comprising at least one of the sequences of SEQ ID NO: 1 to 26 or 53 to 75 or 315 to 318;
   b) a polynucleotide or polynucleotide analog which hybridizes under stringent conditions to a polynucleotide specified in (a) encoding a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c)
   attached to a solid support.
17. A method of screening for agents which regulate the activity of a polypeptide encoded by a polynucleotide or polynucleotide analog selected from the group consisting of:
   a) a polynucleotide or polynucleotide analog comprising at least one of the sequences of SEQ ID NO:2 to 6, 8, 9, 11 to 16, 18, 19, 21 to 26, 53 to 75 or 315 to 318;
   b) a polynucleotide or polynucleotide analog which hybridizes under stringent conditions to a polynucleotide specified in (a) encoding a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c);
   comprising the steps of:
   i) contacting a test compound with at least one polypeptide encoded by a polynucleotide specified in (a) to (d); and
   ii) detecting binding of the test compound to the polypeptide, wherein a test compound which binds to the polypeptide is identified as a potential therapeutic agent for modulating the activity of the polypeptide in order to prevent of treat malignant neoplasia.
18. A method of screening for agents which regulate the activity of a polypeptide encoded by a polynucleotide or polynucleotide analog selected from the group consisting of:
   a) a polynucleotide or polynucleotide analog comprising at least one of the sequences of SEQ ID NO: 2 to 6, 8, 9, 11 to 16, 18, 19, 21 to 26, 53 to 75, or 315 to 318;
   b) a polynucleotide or polynucleotide analog which hybridizes under stringent conditions to a polynucleotide specified in (a) encoding a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c)
   comprising the steps of:
   i) contacting a test compound with at least one polypeptide encoded by a polynucleotide specified in (a) to (d); and
   ii) detecting the activity of the polypeptide as specified for the respective sequence in Table 2 or 3, wherein a test compound which increases the activity is identified as a potential preventive or therapeutic agent for increasing the polypeptide activity in malignant neoplasia, and wherein a test compound which decreases the activity of the polypeptide is identified as a potential therapeutic agent for decreasing the polypeptide activity in malignant neoplasia.
19. A method of screening for agents which regulate the activity of a polynucleotide or polynucleotide analog selected from group consisting of;
   a) a polynucleotide or polynucleotide analog comprising at least one of the sequences of SEQ ID NO: 2 to 6, 8, 9, 11 to 16, 18, 19, 21 to 26, 53 to 75 or 315 to 318;
   b) a polynucleotide or polynucleotide analog which hybridizes under stringent conditions to a polynucleotide specified in (a) encoding a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c)
   comprising the steps of:
   i) contacting a test compound with at least one polynucleotide or polynucleotide analog specified in (a) to (d), and
   ii) detecting binding of the test compound to the polynucleotide, wherein a test compound which binds to the polynucleotide is identified as a potential preventive or therapeutic agent for regulating the activity of the polynucleotide in malignant neoplasia.
20. Use of
   a) a polynucleotide or polynucleotide analog comprising at least one of the sequences of SEQ ID NO: 2 to 6, 8, 9, 11 to 16, 18, 19, 21 to 26, 53 to 75 or 315 to 318;
   b) a polynucleotide which hybridizes under stringent conditions to a polynucleotide or polynucleotide analog specified in (a) encoding a polypeptide exhibiting the same biological function as specified for the respective sequence in Table 2 or 3;
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3;
   d) a polynucleotide or polynucleotide analog which represents a specific fragment, derivative or allelic variation of a polynucleotide sequence specified in (a) to (c);
   e) an antisense molecule targeting specifically one of the polynucleotide sequences specified in (a) to (d);
   f) a purified polypeptide encoded by a polynucleotide or polynucleotide analog sequence specified in (a) to (d)
   g) a purified polypeptide comprising at least one of the sequences of SEQ ID NO: 28 to 32, 34, 35, 37 to 42, 44, 45, 47 to 52, 76 to 98 or 393 to 396;
   h) an antibody capable of binding to one of the polynucleotide specified in (a) to (d) or a polypeptide specified in (f) and (g);
   i) a reagent identified by any of the methods of aspect 17 to 19 that modulates the amount or activity of a polynucleotide sequence specified in (a) to (d) or a polypeptide specified in (f) and (g);
   in the preparation of a composition for the prevention, prediction, diagnosis, prognosis or a medicament for the treatment of malignant neoplasia.
21. Use of aspect 20 wherein the disease is breast cancer.
22. A reagent that regulates the activity of a polypeptide selected from the group consisting of:
   a) a polypeptide encoded by any polynucleotide or polynucleotide analog comprising at least one of the sequences of SEQ ID NO: 2 to 6, 8, 9, 11 to 16, 18, 19, 21 to 26, 53 to 75 or 315 to 318;
   b) a polypeptide encoded by any polynucleotide or polynucleotide analog which hybridizes under stringent conditions to any polynucleotide comprising at least one of the sequences of SEQ ID NO: 2 to 6, 8, 9, 11 to 16, 18, 19, 21 to 26, 53 to 75 or 315 to 318 encoding a polypeptide exhibiting the same biological function as specified for the respective sequence in Table 2 or 3
   c) a polypeptide encoded by any polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polypeptide encoded by any polynucleotide or polynucleotide analog which represents a specific fragment, derivative or allelic variation of a polynucleotide sequence specified in (a) to (c) encoding a polypeptide exhibiting the same biological function as specified for the respective sequence in Table 2 or 3
   e) or a polypeptide comprising at least one of the sequences of SEQ ID NO: 28 to 32, 34, 35, 37 to 42, 44, 45, 47 to 52, 76 to 98 or 393 to 396;
   wherein said reagent is identified by the method of any of the aspects 17 to 19.
23. A reagent that regulates the activity of a polynucleotide or polynucleotide analog selected from the group consisting of:
   a) a polynucleotide or polynucleotide analog comprising at least one of the sequences SEQ ID NO:2 to 6, 8, 9, 11 to 16, 18, 19, 21 to 26, 53 to 75 or 315 to 318;
   b) a polynucleotide or polynucleotide analog which hybridizes under stringent conditions to a polynucleotide specified in (a) encoding a polypeptide exhibiting the same biological function as specified for the respective sequence in Table 2 or 3
   c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
   d) a polynucleotide or polynucleotide analog which represents a specific fragment, derivative or allelic variation of a polynucleotide sequence specified in (a) to (c) encoding a polypeptide exhibiting the same biological function as specified for the respective sequence in Table 2 or 3
   wherein said reagent is identified by the method of any of the aspects 17 to 19.
24. A pharmaceutical composition, comprising:
   a) an expression vector containing at least one polynucleotide or polynucleotide analog selected from the group consisting of:
      i) a polynucleotide or polynucleotide analog comprising at least one of the sequences of SEQ ID NO: 2 to 6, 8, 9, 11 to 16, 18, 19, 21 to 26, 53 to 75 or 315 to 318;
      ii) a polynucleotide or polynucleotide analog which hybridizes under stringent conditions to a polynucleotide specified in (a) encoding a polypeptide exhibiting the same biological function as specified for the respective sequence in Table 2 or 3
      iii) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
      iv) a polynucleotide or polynucleotide analog which represents a specific fragment, derivative or allelic variation of a polynucleotide sequence specified in (a) to (c) encoding a polypeptide exhibiting the same biological function as specified for the respective sequence in Table 2 or 3;
   or the reagent of aspect 22 or 23 and a pharmaceutically acceptable carrier.
25. A computer-readable medium comprising:
   a) at least one digitally encoded value representing a level of expression of at least one polynucleotide sequence of SEQ ID NO: 2 to 6, 8, 9, 11 to 16, 18, 19, 21 to 26, 53 to 75 or 315 to 318
   b) at least 2 digitally encoded values representing the levels of expression of at least 2 polynucleotide sequences selected from SEQ ID NO: 1 to 26, 53 to 75 or 315 to 318
   in a cell from the a subject at risk for or having malignant neoplasia.
26. A method for the detection of chromosomal alterations characterized in that the relative abundance of individual mRNAs, encoded by genes, located in altered chromosomal regions is detected.
27. A method for the detection of chromosomal alterations characterized in that the copy number of one or more chromosomal region(s) is detected by quantitative PCR.

### EXAMPLE 1

### Expression Profiling

a) Expression Profiling Utilizing Quantitative RT-PCR

For a detailed analysis of gene expression by quantitative PCR, methods, one will utilize primers flanking the genomic region of interest and a fluorescent labeled probe hybridizing in-between. Using the PRISM 7700 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly in the 3′ region of the coding sequence or in the 3′ untranslated region (see Table 5 for primer- and probe-sequences) according to the relative positions of the probe sequence used for the construction of the Affymetrix HG_U95A-E or HG-U133A-B DNA-chips. All primer pairs were checked for specificity by conventional PCR reactions. To standardize the amount of sample RNA, GAPDH was selected as a reference, since it was not differentially regulated in the samples analyzed. TaqMan validation experiments were performed showing that the efficiencies of the target and the control amplifications are approximately equal which is a prerequisite for the relative quantification of gene expression by the comparative ΔΔC_{T} method, known to those with skills in the art.

As well as the technology provided by Perkin Elmer one may use other technique implementations like Lightcycler™ from Roche Inc. or iCycler from Stratagene Inc.

b) Expression Profiling Utilizing DNA Microarrays

Expression profiling can bee carried out using the Affymetrix Array Technology. By hybridization of mRNA to such a DNA-array or DNA-Chip, it is possible to identify the expression value of each transcripts due to signal intensity at certain position of the array. Usually these DNA-arrays are produced by spotting of cDNA, oligonucleotides or subcloned DNA fragments. In case of Affymetrix technology app. 400.000 individual oligonucleotide sequences were synthesized on the surface of a silicon wafer at distinct positions. The minimal length of oligomers is 12 nucleotides, preferable 25 nucleotides or full length of the questioned transcript. Expression profiling may also be carried out by hybridization to nylon or nitro-cellulose membrane bound DNA or oligonucleotides. Detection of signals derived from hybridization may be obtained by either colorimetric, fluorescent, electrochemical, electronic, optic or by radioactive readout. Detailed description of array construction have been mentioned above and in other patents cited. To determine the quantitative and qualitative changes in the chromosomal region to analyze, RNA from tumor tissue which is suspected to contain such genomic alterations has to be compared to RNA extracted from benign tissue (e.g. epithelial breast tissue, or micro dissected ductal tissue) on the basis of expression profiles for the whole transcriptome. With minor modifications, the sample preparation protocol followed the Affymetrix GeneChip Expression Analysis Manual (Santa Clara, Calif.). Total RNA extraction and isolation from tumor or benign tissues, biopsies, cell isolates or cell containing body fluids can be performed by using TRIzol (Life Technologies, Rockville, Md.) and Oligotex mRNA Midi kit (Qiagen, Hilden, Germany), and an ethanol precipitation step should be carried out to bring the concentration to 1 mg/ml. Using 5-10 mg of mRNA to create double stranded cDNA by the SuperScript system (Life Technologies). First strand cDNA synthesis was primed with a T7-(dT24) oligonucleotide. The cDNA can be extracted with phenol/chloroform and precipitated with ethanol to a final concentration of 1 mg/ml. From the generated cDNA, CRNA can be synthesized using Enzo's (Enzo Diagnostics Inc., Farmingdale, N.Y.) in vitro Transcription Kit. Within the same step the cRNA can be labeled with biotin nucleotides Bio-11-CTP and Bio-16-UTP (Enzo Diagnostics Inc., Farmingdale, N.Y.). After labeling and cleanup (Qiagen, Hilden (Germany) the cRNA then should be fragmented in an appropriated fragmentation buffer (e.g., 40 mM Tris-Acetate, pH 8.1, 100 mM KOAc, 30 mM MgOAc, for 35 minutes at 94° C.). As per the Affymetrix protocol, fragmented cRNA should be hybridized on the HG_U133 arrays A and B, comprising app. 40.000 probed transcripts each, for 24 hours at 60 rpm in a 45° C. hybridization oven. After Hybridization step the chip surfaces have to be washed and stained with streptavidin phycoerythrin (SAPE; Molecular Probes, Eugene, Oreg.) in Affymetrix fluidics stations. To amplify staining, a second labeling step can be introduced, which is recommended but not compulsive. Here one should add SAPE solution twice with an antistreptavidin biotinylated antibody. Hybridization to the probe arrays may be detected by fluorometric scanning (Hewlett Packard Gene Array Scanner; Hewlett Packard Corporation, Palo Alto, Calif.).

After hybridization and scanning, the microarray images can be analyzed for quality control, looking for major chip defects or abnormalities in hybridization signal. Therefor either Affymetrix GeneChip MAS 5.0 Software or other microarray image analysis software can be utilized. Primary data analysis should be carried out by software provided by the manufacturer.

In case of the genes analyses in one embodiment of this invention the primary data have been analyzed by further bioinformatic tools and additional filter criteria. The bioinformatic analysis is described in detail below.

c) Data Analysis

According to Affymetrix measurement technique (Affymetrix GeneChip Expression Analysis Manual, Santa Clara, Calif.) a single gene expression measurement on one chip yields the average difference value and the absolute call. Each chip contains 16-20 oligonucleotide probe pairs per gene or cDNA clone. These probe pairs include perfectly matched sets and mismatched sets, both of which are necessary for the calculation of the average difference, or expression value, a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The average difference is a numeric value supposed to represent the expression value of that gene. The absolute call can take the values ‘A’ (absent), ‘M’ (marginal), or ‘P’ (present) and denotes the quality of a single hybridization. We used both the quantitative information given by the average difference and the qualitative information given by the absolute call to identify the genes which are differentially expressed in biological samples from individuals with breast cancer versus biological samples from the normal population. With other algorithms than the Affymetrix one we have obtained different numerical values representing the same expression values and expression differences upon comparison.

The differential expression E in one of the breast cancer groups compared to the normal population is calculated as follows. Given n average difference values d₁, d₂, . . . , dₙ in the breast cancer population and m average difference values c₁, c₂, . . . , cₘ in the population of normal individuals, it is computed by the equation:

$$E ≡ exp ( \frac{1}{m} {\sum }_{i = 1}^{m} ln ( {c}_{i} ) - \frac{1}{n} {\sum }_{i = 1}^{n} ln ( {d}_{i} ) )$$

If dⱼ<50 or cᵢ<50 for one or more values of i and j, these particular values cᵢ and/or dⱼ are set to an “artificial” expression value of 50. These particular computation of E allows for a correct comparison to TaqMan results.

A gene is called up-regulated in breast cancer versus normal if E≧1.5 and if the number of absolute calls equal to ‘P’ in the breast cancer population is greater than n/2.

A gene is called down-regulated in breast cancer versus normal if E≦1.5 and if the number of absolute calls equal to ‘P’ in the normal population is greater than m/2.

The final list of differentially regulated genes consists of all up-regulated and all down-regulated genes in biological samples from individuals with breast cancer versus biological samples from the normal population. Those genes on this list which are interesting for a pharmaceutical application were finally validated by TaqMan. If a good correlation between the expression values/behavior of a transcript could be observed with both techniques, such a gene is listed in Tables 1 to 3.

Since not only the information on differential expression of a single gene within an identified ARCHEON, but also the information on the co-regulation of several members is important for predictive, diagnostic, preventive and therapeutic purposes we have combined expression data with information on the chromosomal position (e.g. golden path) taken from public available databases to develop a picture of the overall transcription of a given tumor sample. By this technique not only known or suspected regions of genomes can be inspected but even more valuable, new regions of disregulation with chromosomal linkage can be identified. This is of value in other types of neoplasia or viral integration and chromosomal rearrangements. By SQL based database searches one can retrieve information on expression, qualitative value of a measurement (denoted by Affymetrix MAS 5.0 Software), expression values derived from other techniques than DNA-chip hybridization and chromosomal linkage.

### EXAMPLE 2

### Identification of the ARCHEON

a) Identification and Localization of Genes or Gene Probes (Represented by the so Called Probe Sets on Affymetrix Arrays Hg-U95A-E or Hg-U133A-B) in their Chromosomal Context and Order on the Human Genome.

For identification of larger chromosomal changes or aberrations, as they have been described in detail above, a sufficient number of genes, transcripts or DNA-fragments is needed. The density of probes covering a chromosomal region is not necessarily limited to the transcribed genes, in case of the use of array based CGH but by utilizing RNA as probe material the density is given by the distance of genes on a chromosome. The DNA-microarrays provided by Affymetrix Inc. Do contain hitherto all transcripts from the known humane genome, which are be represented by 40.000-60.000 probe sets. By BLAST mapping and sorting the sequences of these short DNA-oligomers to the public available sequence of the human genome represented by the so called “golden path”, available at the university of California in Santa Cruz or from the NCBI, a chromosomal display of the whole Transcriptome of a tissue specimen evolves. By graphical display of the individual chromosomal regions and color coding of over or under represented transcripts, compared to a reference transcriptome regions with DNA gains and losses can be identified.

b) Quantification of Gene Copy Numbers by Combined IHC and Quantitative PCR (PCR Karyotyping) or Directly by Quantitative PCR

Usually one to three paraffin-embedded tissue sections that are 5 μm thick are used to obtain genomic DNA from the samples. Tissue section are stained by colorimetric IHC after deparaffinization to identify regions containing disease associated cells. Stained regions are macrodissected with a scalpel and transferred into a micro-centrifuge tube. The genomic DNA of these isolated tissue sections is extracted using appropriate buffers. The isolated DNA is then used for quantitative PCR with appropriate primers and probes. Optionally the IHC staining can be omitted and the genomic DNA can be directly isolated with or without prior deparaffinization with appropriate buffers. Those who are skilled in the art may vary the conditions and buffers described below to obtain equivalent results.

Reagents from DAKO (HercepTest Code No. K 5204) and TaKaRa were used (Biomedicals Cat.: 9091) according to the manufactures protocol.

It is convenient to prepare the following reagents prior to staining:

### Solution No. 7

Epitope Retrieval Solution (Citrate buffer+antimicrobial agent) (10× conc.)

20 ml ad 200 ml aqua dest. (stable for Imonth at 2-8° C.)

### Solution No. 8

Washing-buffer (Tris-HCl+antimicrobial agent) (10× conc.)

30 ml ad 300 ml destined water (stable for 1 month at 2-8° C.)

Staining solution: DAB

1 ml solution is sufficient for 10 slides. The solution were prepared immediately before usage:

1 ml DAB buffer (Substrate Buffer solution, pH 7.5, containing H₂O₂, stabilizer, enhancers and an antimicrobial agent)+1 drop (25-3 μl) DAB-Chromogen (3,3′-diaminobenzidine chromogen solution). This solution is stable for up to 5 days at 2-8° C. Precipitated substances do not influence the staining result. Additionally required are: 2× approx. 100 ml Xylol, 2× approx. 100 ml Ethanol 100%, 2× Ethanol 95%, aqua dest. These solution can be used for up to 40 stainings. A water bath is required for the epitope retrieval step.

### Staining Procedure:

All reagents are pre-warmed to room temperature (20-25° C.) prior to immunostaining; Likewise all incubations were performed at room temperature. Except the epitope retrieval which is performed in at 95° C. water bath. Between the steps excess of liquid is tapped off from the slides with lintless tissue (Kim Wipe).

Deparaffinization

Slides are placed in a xylene bath and incubated for 5 minutes. The bath is changed and the step repeated once. Excess of liquid is tapped off and the slides are placed in absolute ethanol for 3 minutes. The bath is changed and the step repeated once. Excess of liquid is tapped off and the slides are placed in 95% ethanol for 3 minutes. The bath is changed and the step repeated once. Excess of liquid is tapped off and the slides are placed in distilled water for a minimum of 30 seconds.

### Epitone Retrival

Staining jars are filled with diluted epitope retrieval solution and preheated in a water bath at 95° C. The deparaffinized sections are immersed into the preheated solution in the staining jars and incubated for 40 minutes at 95° C. The entire jar is removed from the water bath and allowed to cool down at room temperature for 20 minutes. The epitope retrieval solution is decanted, the sections are rinsed in distilled water and finally soaked in wash buffer for 5 minutes.

### Peroxidase Blocking:

Excess of buffer is tapped off and the tissue section encircled with a DAKO pen. The specimen is covered with 3 drops (100 μl) Peroxidase-Blocking solution and incubated for 5 minutes. The slides are rinsed in distilled water and placed into a fresh washing buffer bath.

### Antibody Incubation

Excess of liquid is tapped off and the specimen are covered with 3 drops (100 μl) of Anti-Her-2/neu reagent (Rabbit Anti-Human Her2 Protein in 0.05 mol/L Tris/HCl, 0.1 mol/L NaCl, 15 mmol/L pH7.2 NaN₃ containing stabilizing protein) or negative control reagent (=IGG fraction of normal rabbit serum at an equivalent protein concentration as the Her2 Ab). After 30 minutes of incubation the slide is rinsed in water and placed into a fresh water bath.

### Visualization

Excess of liquid is tapped off and the specimen are covered with 3 drops (100 μl) of visualization reagent. After 30 minutes of incubation the slide is rinsed in water and placed into a fresh water bath. Excess of liquid is tapped off and the specimen are covered with 3 drops (100 μl) of Substrate-Chromogen solution (DAB) for 10 minutes. After rinsing the specimen with distalled water, photographs are taken with a conventional Olympus microscope to document the staining intensity and tumor regions within the specimen. Optionally a counterstain with hematoxylin was performed.

### DNA Extraction

The whole specimens or dissected subregions are transferred into a microcentrifuge tubes. Optionally a small amount (10 μl) of preheated TaKaRa solution (DEXPAT™) is preheated and placed onto the specimen to facilitate sample transfer with a scalpel. 50 to 150 μl of TaKaRa solution were added to the samples depending on the size of the tissue sample selected. The sample are incubated at 100° C. for 10 minutes in a block heater, followed by centrifugation at 12.000 rpm in a microcentrifuge. The supernatant is collected using a micropet and placed in a separate microcentrifuge tube. If no deparaffinization step has been undertaken one has to be sure not to withdraw tissue debris and resin. Genomic DNA left in the pellet can be collected by adding resin-free TaKaRa buffer and an additional heating and centrifugation step. Samples are stored at −20° C.

Genomic DNA from different tumor cell lines (MCF-7, BT-20, BT474, SKBR-3, AU-565, UACC-812, UACC-893, HCC-1008, HCC-2157, HCC-1954, HCC-2218, HCC-1937, HCC1599, SW480), or from lymphocytes is prepared with the QIAamp™ DNA Mini Kits or the QIAamp® DNA Blood Mini Kits according to the manufacturers protocol. Usually between 1 ng up to 1 μg DNA is used per reaction.

### Quantitative PCR

To measure the gene copy number of the genes within the patient samples the respective primer/probes (see table below) are prepared by mixing 25 μl of the 100 μM stock solution “Upper Primer”, 25 μl of the 100 μM stock solution “Lower Primer” with 12.5 μl of the 100 μM stock solution Taq Man Probe (Quencher Tamra) and adjusted to 500 μl with aqua dest. For each reaction 1.25 μl DNA-Extract of the patient samples or 1.25 μl DNA from the cell lines were mixed with 8.75 μl nuclease-free water and added to one well of a 96 Well-Optical Reaction Plate (Applied Biosystems Part No. 4306737). 1.5 μl Primer/Probe mix, 12, μl Taq Man Universal-PCR Mix (2×) (Applied Biosystems Part No. 4318157) and 1 μl Water are then added. The 96 well plates are closed with 8 Caps/Strips (Applied Biosystems Part Number 4323032) and centrifuged for 3 minutes. Measurements of the PCR reaction are done according to the instructions of the manufacturer with a TaqMan 7900 HT from Applied Biosystems (No. 20114) under appropriate conditions (2 min. 50° C., 10 min. 95° C., 0.15 min. 95° C., 1 min. 60° C.; 40 cycles). SoftwareSDS 2.0 from Applied Biosysrtems is used according to the respective instructions. CT-values are then further analyzed with appropriate software (Microsoft Excel™).

### EXAMPLE 3

Clinical Samples of patients being treated with Herceptin and a chemotherapeutic agent (e.g. docetaxel, paclitaxel, taxotere, carboplatin, cisplatin, oxaliplatin, vinorelbine) as a second line therapy have been obtained. These samples included formalin-fixed and paraffin-embedded material from primary tumours and metastatic lesions of the respective patients. However, the determination of the ARCHEON genes as disclosed in this invention, has also been performed from fresh tissue after nucleic acid extraction in an independent, neoadjuvant setting. Moreover, whole blood, serum and plasma samples were available for multiple patients.

Multiparametric, clinical assessment of the response to Herceptin in combination with chemotherapeutics (e.g. docetaxel, taxotere, paclitaxel, vinorelbine, carboplatin, cisplatin), or other therapies described below, was performed. Clinical information included histological parameters (INM-Stage, AJCC grade), standard molecular markers (IHC staining for estrogen receptor, progesteron receptor, Her-2/neu) and sonographical or radiological assessment (e.g. CT). Response to treatment was evaluated according to international standards, i.e. modified WHO criteria and RECIST criteria. Each cancer evaluation in the course of the disease was documented (method and date of evaluation, organ, anatomical description, measurability, size of lesion (longest diameter), greatest perpendicular diameter, tumor area). Moreover, each systemic anticancer therapy including prior chemotherapy with anthracyclins (Doxorubicin or Epirubicin) and/or CMF and the response thereto was evaluated (drug, intent, duration, schedule, number of cycles, cumulative dose). The response to combinatory treatment of metatstatic breast cancer patients with Herceptin and chemotherapeutica as second line treatment the modified WHO criteria were used. In addition the initial disease free survival, duration of response and time to progreesion were taken into consideration. For definition of treatment response standard criteria were used: “Complete Response” (“CR”=tumor shrinkage of 100% with no residual disease being clinical detectable), “Partial Response” (“PR”=tumor shrinkage of target lesion of at least 50%), “Stable Disease” (“SD”=tumor shrinkage of less than 50% or no change) and “Progressive Disease” (“PD” tumor growth or new tumor lesions).

More than 70 genes were analyzed according to the method disclosed in example 2 by combined IHC and quantitative PCR or directly by quantitative PCR after nucleic acid extraction from the formaldehyde-fixed, paraffin-embedded tissue slides. Results were reconfirmed by independent methodology (VNTR and SNP detection). Alterations of the 43 ARCHEON genes were determined by comparison with reference genes, that are located on the same chromosome (=intrachromosomal control,) or different chromosomes (=extrachromosomal control). Intrachromosomal reference genes included MMP28, hKa3 and K20. Extrachromosomal reference genes included GAPDH for chromosome 12. However any other gene not included in the ARCHEONs disclosed in this invention can be used as reference gene for ARCHEON characterization. The reference genes should be independent from the ARCHEON alterations occurring in the neoplastic lesions and should be not affected by chromosomal alterations such as amplifications and deletions. As gene copy numbers of non-amplified genes can be increased in neoplastic lesions due to genomic imbalances such as aneuploidie or polyploidie, each measurement of ARCHEON genes was correlated to multiple reference genes to minimize the influence of genomic imbalances on the relative copy number calculation. Moreover, minor systemic errors occurring due to differences in the performance of individual primer/probe pairs were minimized by determining primer/probe performances in control tissues (i.e. non-neoplastic tissues from healthy controls) and euploid control cell lines (e.g. HS68, ATCC #CRL1635). Moreover one well characterized, control cell line was used, that displays aneuploidie for a single chromosome (i.e. Detroit, ATCC#CCL-54; trisomie 21). By measuring genes located on the X-chromosome (e.g. SRY), the Y-chromosome (e.g. Xist) and on chromosome 21, defined copy numbers of 1, 2 and 3 genes could be determined as internal control during each run for standardization. In addition, synthetic targets were spiked into some reactions, that consisted of the target region of the PCR forward and reverse primers of the gene to be normalized, but in between consisted of a synthetic probe hybridization region different from the original probe region of the target gene to be normalized. This allowed internal standardization of each individual qPCR reaction by multiplex PCR. The calculated performance differences were used as a filter for the measurements within the target tissues, i.e. primer/probe differences of each individual gene as depicted in the control cells and tissues were subtracted from each individual gene measurement performed in the target tissue. Thereafter, the individual, filtered CT values were normalized to the different reference genes. Differences between the CT values of the quantitative PCR reactions of the ARCHEON genes and the reference genes remaining after filtering the primer/probe performance differences were determined and transformed into “copy numbers per cell”. This was done by subtracting the CT values of the target genes from the CT values of the reference genes. The resulting ΔCT values were then transformed in gene copy numbers, with the ΔCT value of the reference gene (ΔCT=0) being defined as “2 copies per cell”, by the following formula: 2*(2̂(ΔCT*(−1))). All the calculations were done using standard software (Microsoft Excel™).

### REFERENCES

### Patents Cited

U.S. Pat. No. 4,843,155 Chomezynski, P.
U.S. Pat. No. 5,262,31 Liang, P., and Pardee, A. B., 1993
U.S. Pat. No. 4,683,202 Mullis, K. B., 1987
U.S. Pat. No. 5,593,839
U.S. Pat. No. 5,578,832
U.S. Pat. No. 5,556,752
U.S. Pat. No. 5,631,734
U.S. Pat. No. 5,599,695
U.S. Pat. No. 4,683,195
U.S. Pat. No. 5,498,531
U.S. Pat. No. 5,714,331
U.S. Pat. No. 5,641,673 Haseloff et al.,
U.S. Pat. No. 5,223,409 Lander, E.,
U.S. Pat. No. 5,976,813 Beutel et al.
U.S. Pat. No. 5,283,317
U.S. Pat. No. 6,203,987
U.S. Pat. No. 6,379,895
WO 97/29212
WO 97/27317
WO 95/22058
WO 99/12826
WO 97/02357
WO 94/13804
WO 94/10300
WO 97/14028
WO 99/52708
EP 0 785 280
EP 0 799 897
EP O 728520
EP 0 721 016
EP 0 321 201
GB2188638B

### Publications Cited

(1) Gusterson et al., Journal of Clinical Oncology 10, 1049-1056, 1992
(2) Achuthan et al., Cancer Genet Cytogenet. 130:166-72, 2001
(3) Tomasetto et al., FEBS Lett. 373: 245-249, 1995
(4) Pragnell et al., FEBS Lett. 291: 253-258, 1991
(5) Nakamichi et al. 1986
(6) Feo et al., Proc. Nat. Acad. Sci. 86: 6691-6695, 1989
(7) Davies et al., Proc. Nat. Acad. Sci. 86: 6691-6695, 1989
(8) Lee et al., Molec. Endocr. 9: 243-254, 1995
(9) Drane et al., Oncogene 15: 3013-3024, 1997
(10) Zhu et al., J. Biol. Chem. 272: 25500-25506, 1997
(11) Yuan et al., Proc. Nat. Acad. Sci. 95: 7939-7944, 1998
(12) Zhu et al., Proc. Nat. Acad. Sci. 96: 10848-10853, 1999
(13) Lee et al., Science 268: 836-844, 1995
(14) McCormick et al., Molec. Cell. Biol. 16: 5792-5800, 1996
(15) Tamimi et al., Genomics 40: 355-357, 1997
(16) Valle et al., FEBS Lett. 415: 163-168, 1997
(17) Kaneda et al., J. Biol. Chem. 263: 7672-7677, 1988
(18) Hoehe et al., Hum. Molec. Genet. 1: 175-178, 1992
(19) Yang-Feng et al., Abstract Cytogenet. Cell Genet. 40: 784, 1985
(20) Coussens et al., Science 230: 1132-1139, 1985
(21) van de Vijver et al., New Eng. J. Med. 319: 1239-1245, 1988
(22) Slamon et al., Science 244: 707-712, 1989
(23) Fukushige et al., Res. Commun. 134: 477-483, 1986
(24) Kaneko et al., Jpn. 3; Cancer Res. 78: 16-19, 1987
(25) Di Fiore et al., Science 237: 178-182, 1987
(26) Popescu et al., Genomics 4: 362-366, 1989.
(27) Qiu et al., Nature 393: 83-85, 1998
(28) Yu et al., Molec. Cell 2: 581-591, 1998
(29) Doherty et al., Proc. Nat. Acad. Sci. 96: 10869-10874, 1999
(30) Slamon et al., New Eng. J. Med. 344: 783-792, 2001
(31) Margolis et al., J. Clin. Invest. 102: 821-827, 1998
(32) Tanaka et al., J. Clin. Invest. 102: 821-827, 1998
(33) Dong et al., J. Biol. Chem. 272: 29104-29112, 1997
(34) Stein et al., EMBO J. 13:1331-40, 1994 (35) Nagata et al., Nature 319: 415-418, 1986
(36) Le Beau et al., Leukemia 1: 795-799, 1987,
(37) Jansson et al., EMBO J. 2: 561-565, 1983
(38) Thompson et al., Science 237:1610-1614, 1987
(39) Nakai et al., Proc. Nat. Acad. Sci. 85: 2781-2785, 1988
(40) Miyajima et al., Cell 57: 31-39, 1989
(41) Debuire et al., Science 224: 1456-1459, 1984
(42) Petkovich et al., Nature 330: 444-450, 1987
(43) Mattei et al., Hum Genet. 80: 186-188, 1988.
(44) Williams et al., Molec. Cell. Biol. 18: 2758-2767, 1998
(45) Saha et al, Molec. Cell. Biol. 18: 2758-2767, 1998
(46) Yan et al., Proc. Nat. Acad. Sci. 95: 3603-3608, 1998
(47) Singh et al., Nucleic Acids Res. 16: 3919-3929, 1988
(48) Tsai-Pflugfelder et al., Proc. Nat. Acad. Sci. 85: 7177-7181, 1988
(49) Chung et al., Proc. Nat. Acad. Sci. 86: 9431-9435, 1989
(50) Lang et al., Gene 221: 255-266, 1998
(51) Keith et al., Genes Chromosomes Cancer 4: 169-175, 1992
(52) Kingsmore et al., Mammalian Genome 4: 288-289, 1993
(53) Watt et al., Biochem. J. 303: 681-695, 1994
(54) Kiefer et al., J. Biol. Chem. 267: 12692-12699, 1992
(55) Shimasaki et al., Molec. Endocr. 4: 1451-1458, 1990
(56) Zazzi et al., Genomics 49: 401-410, 1998
(57) Bajalica et al., Hum. Genet. 89: 234-236, 1992
(58) Tonin et al., Genomics 18: 414-417, 1993
(59) Birkenbach et al., J. Virol. 67: 2209-2220, 1993
(60) Wang et al., Proc. Nat. Acad. Sci. 95: 492-498, 1998
(61) Klochendler-Yeivin et al., Curr Opin Genet Dev 121:73-9, 2002
(62) Ring et al., Genomics 51:140-3, 1998
(63) Darmon et al., Molec. Biol. Rep. 12: 277-283, 1987
(64) Zhou et al., J. Biol. Chem. 263: 15584-15589, 1988
(65) Korge et al., Proc. Nat. Acad. Sci. 89: 910-914, 1992
(66) Lessin et al., J. Invest. Derim 91: 572-578, 1988
(67) Romano et al., Cytogenet. Cell Genet. 58: 2009-2010, 1991.
(68) Fuchs et al., Proc. Nat. Acad. Sci. 89: 6906-6910, 1992
(69) Rogaev et al., Nature Genet. 5: 158-162, 1993
(70) Liu et al., Curr. Eye Res. 12: 963-974, 1993
(71) Nishida et al., Invest Ophthal. Vis. Sci. 37: 1800-1809, 1996
(72) Nishida et al., Am. J. Hum Genet. 61: 1268-1275, 1997
(73) Meesmann and Wilke, 1939
(74) Corden et al., Exp. Eye Res. 70: 4149, 2000
(75) van de Vijver et al., Mol Cell Biol 7, 2091-23, 1987
(76) Offterdinger et al., Biochem Biophys Res Comm 251, 907-13, 1988
(77) Sambrook et al., MOLECULAR CLONING: A LABORATORY MANUAL, 2d ed., 1989
(78) Ausubel et al., CURRENT PROTOCOLS IN MOLECULAR BIOLOGY, John Wiley & Sons, New York, N.Y., 1989.
(79) Tedder, T. F. et al., Proc. Natl. Acad. Sci. U.S.A. 85:208-212, 1988
(80) Hedrick, S. M. et al., Nature 308:149-153, 1984
(81) Lee, S. W. et al., Proc. Natl. Acad. Sci. U.S.A. 88:4225, 1984
(82) Sarkar, PCR Methods Applic. 2, 318-322, 1993
(83) Triglia et al., Nucleic Acids Res. 16, 81-86, 1988
(84) Lagerstrom et al., PCR Methods Applic. 1, 111-119, 1991
(85) Copeland & Jenkins, Trends in Genetics 7: 113-118, 1991
(86) Cohen, et al., Nature 366: 698-701, 1993
(87) Bonner et al., J. Mol. Biol. 81, 123 1973
(88) Bolton and McCarthy, Proc. Natl. Acad. Sci. U.S.A. 48, 1390 1962
(89) Plump et al., Cell 71: 343-353, 1992
(90) Altschul et al., Bull. Math. Bio. 48:603, 1986,
(91) Henikoff & Henikoff, Proc. Natl. Acad. Sci. USA 89:10915, 1992
(92) Pearson & Lipman, Proc. Nat'l. Acad. Sci. USA 85:2444, 1988
(93) Pearson et al., Meth. Enzymol. 183:63, 1990
(94) Needleman & Wunsch, J. Mol. Biol. 48:444, 1970
(95) Sellers, SIAM J. Appl. Math. 26:787, 1974
(96) Takamatsu, EMBO J. 6, 307-311, 1987
(97) Coruzzi et al., EMBO J. 3, 1671-1680, 1984
(98) Broglie et al., Science 224, 838-843, 1984
(99) Winter et al., Results Probl. Cell Differ. 17, 85-105, 1991
(100) Engelhard et al., Proc. Nat. Acad. Sci. 91, 3224-3227, 1994
(101) Logan & Shenk, Proc. Natl. Acad. Sci. 81, 3655-3659, 1984
(102) Scharf et al., Results Probl. Cell Differ. 20, 125-162, 1994
(103) Freshney R. I., ed., ANIMAL CELL CULTURE, 1986
(104) Wigler et al., Cell 11, 223-232, 1977
(105) Lowy et al., Cell 22, 817-823, 1980
(106) Wigler et al., Proc. Natl. Acad. Sci. 77, 3567-3570, 1980
(107) Colbere-Garapin et al., J. Mol. Biol. 150, 114, 1981
(108) Hartman & Mulligan, Proc. Natl. Acad. Sci. 85, 8047-8051, 1988
(109) Rhodes et al., Methods Mol. Biol. 55, 121-131, 1995
(110) Hampton et al., SEROLOGICAL METHODS: A LABORATORY MANUAL, APS Press, St. Paul, Minn., 1990

(111) Maddox et al., J. Exp. Med. 158, 1211-1216, 1983
(112) Porath et al., Prot. Exp. Purif. 3, 263-281, 1992
(113) Kroll et al., DNA Cell Biol. 12, 441-453, 1993
(114) Caruthers et al., Nucl. Acids Res. Symp. Ser. 215-223, 1980
(115) Horn et al. Nucl. Acids Res. Symp. Ser. 225-232, 1980
(116) Merrifield, J. Am. Chem. Soc. 85, 2149-2154, 1963
(117) Roberge et al., Science 269, 202-204, 1995
(118) Creighton, PROTEINS: STRUCTURES AND MOLECULAR PRINCIPLES, WH and Co., New York, N.Y., 1983
(119) Cronin et al., Human Mutation 7:244, 1996
(120) Landegran et al., Science 241:1077-1080, 1988
(121) Nakazawa et al., PNAS 91:360-364, 1994
(122) Abravaya et al., Nuc Acid Res 23:675-682, 1995
(123) Guatelli, J. C. et al., Proc. Natl. Acad. Sci. USA 87:1874-1878, 1990
(124) Kwoh, D. Y. et al., Proc. Natl. Acad. Sci. USA 86:1173-1177, 1989
(125) Lizardi, P. M. et al., Bio/Technology 6:1197, 1988
(126) Brown, Meth. Mol. Biol. 20, 18, 1994
(127) Sonveaux, Meth. Mol. Biol. 26, 1-72, 1994
(128) Uhlmann et al., Chem. Rev. 90, 543-583, 1990
(129) Gee et al., in Huber & Carr, MOLECULAR AND IMMUNOLOGIC APPROACHES, Publishing Co., Mt. Kisco, N.Y., 1994
(130) Agrawal et al., Trends Biotechnol. 10, 152-158, 1992
(131) Uhlmann et al., Tetrahedron. Lett. 215, 3539-3542, 1987
(132) Cech, Science 236, 1532-1539, 1987
(133) Cech, Ann. Rev. Biochem. 59, 543-568, 1990
(134) Couture & Stinchcomb, Trends Genet. 12, 510-515, 1996
(135) Haseloff et al. Nature 334, 585-591, 1988
(136) Kohler et al., Nature 256, 495-497, 1985
(137) Kozbor et al., J. Immunol. Methods 81, 3142, 1985
(138) Cote et al., Proc. Natl. Acad. Sci. 80, 2026-2030, 1983
(139) Cole et al., Mol. Cell. Biol. 62, 109-120, 1984
(140) Morrison et al., Proc. Natl. Acad. Sci. 81, 6851-6855, 1984
(141) Neuberger et al., Nature 312, 604-608, 1984
(142) Takeda et al., Nature 314, 452-454, 1985
(143) Burton, Proc. Natl. Acad. Sci. 88, 11120-11123, 1991
(144) Thirion et al., Eur. J. Cancer Prev. 5, 507-11, 1996
(145) Coloma & Morrison, Nat. Biotechnol. 15, 159-63, 1997
(146) Mallender & Voss, J. Biol. Chem. 269, 199-206, 1994
(147) Verhaar et al., Int. J. Cancer 61, 497-501, 1995
(148) Nicholls et al., J. Immunol. Meth. 165, 81-91, 1993
(149) Orlandi et al., Proc. Natl. Acad. Sci. 86, 3833-3837, 1989
(150) Winter et al., Nature 349, 293-299, 1991
(151) Lam, Anticancer Drug Des. 12, 145, 1997
(152) DeWitt et al., Proc. Natl. Acad. Sci. U.S.A. 90, 6909, 1993
(153) Erb et al. Proc. Natl. Acad. Sci. U.S.A. 91, 11422, 1994
(154) Zuckermann et al., J. Med. Chem. 37, 2678, 1994
(155) Cho et al., Science 261, 1303, 1993
(156) Carell et al., Angew. Chem. Int. Ed. Engl. 33, 2059 & 2061, 1994
(157) Gallop et al., J. Med. Chem. 37, 1233, 1994
(158) Houghten, BioTechniques 13, 412-421, 1992
(159) Lam, Nature 354, 8284, 1991
(160) Fodor, Nature 364, 555-556, 1993
(161) Cull et al., Proc. Natl. Acad. Sci. U.S.A. 89, 1865-1869, 1992
(162) Scott & Smith, Science 249, 386-390, 1990
(163) Devlin, Science 249, 404-406, 1990
(164) Cwirla et al., Proc. Natl. Acad. Sci. 97, 6378-6382, 1990
(165) Felici, J. Mol. Biol. 222, 301-310, 1991
(166) Jayawickreme et al., Proc. Natl. Acad. Sci. U.S.A. 19, 1614-1618, 1994
(167) Chelsky, Strategies for Screening Combinatorial Libraries 1995
(168) Salmon et al., Molecular Diversity 2, 57-63, 1996
(169) McConnell et al., Science 257, 1906-1912, 1992
(170) Sjolander & Urbaniczky, Anal. Chem. 63, 2338-2345, 1991
(171) Szabo et al., Curr. Opin. Struct. Biol. 5, 699-705, 1995
(172) Zervos et al., Cell 72, 223-232, 1993
(173) Madura et al., J. Biol. Chem. 268, 12046-12054, 1993
(174) Bartel et al., BioTechniques 14, 920-924, 1993
(175) Iwabuchi et al., Oncogene 8, 1693-1696, 1993
(176) Findeis et al. Trends in Biotechnol. 11, 202-205, 1993
(177) Chiou et al., GENE THERAPEUTICS: METHODS AND APPLICATIONS OF DIRECT GENETRANSFER J. A. Wolff, ed., 1994
(178) Wu & Wu, J. Biol. Chem. 263, 621-24, 1988
(179) Wu et al., J. Biol. Chem. 269, 542-46, 1994
(180) Zenke et al., Proc. Natl. Acad. Sci. U.S.A. 87, 3655-59, 1990
(181) Wu et al., J. Biol. Chem. 266, 33842, 1991
(182) REMINGTON'S PHARMACEUTICAL SCIENCES Maack Publishing Co., Easton, Pa.
(183) Hille, Excitable Membranes, Sunderland, Mass., Sinauer Associates, Inc.

TABLE 1

| DNA | Protein | | | | |
|---|---|---|---|---|---|
| SEQ ID NO: | SEQ ID NO: | Genbank ID | Unigene_v162_ID | Locus Link ID | Gene Name |
| 1 | 27 | NM_006148.1 | Hs.334851 | 3927 | LASP1 |
| 2 | 28 | NM_000723.1 | Hs.635 | 782 | CACNB1 |
| 3 | 29 | NM_000981.1 | Hs.381061 | 6143 | RPL19 |
| 4 | 30 | Y13467 | Hs.15589 | 5469 | PPARGBP |
| 5 | 31 | NM_016507.1 | Hs.416108 | 51755 | CrkRS/CRK7 |
| 6 | 32 | AB021742.1 | Hs:322431 | 4761 | NEUROD2 |
| 7 | 33 | NM_006804.1 | Hs.77628 | 10948 | MLN64/STARD3 |
| 8 | 34 | NM_003673.1 | Hs.343603 | 8557 | TELETHONIN |
| 9 | 35 | NM_002686.1 | Hs.1892 | 5409 | PNMT |
| 10 | 36 | X03363.1 | Hs.446352 | 2064 | ERBB2 |
| 11 | 37 | AB008790.1 | Hs.86859 | 2886 | GRB7 |
| 12 | 38 | NM_002809.1 | Hs.9736 | 5709 | PSMD3 |
| 13 | 39 | NM_000759.1 | Hs.2233 | 1440 | GCSFG/CSF3 |
| 14 | 40 | AI023317 | Hs.23106 | 9862 | KIAA0130/ |
| | | NM_014815 | | | TRAP100 |
| 15 | 41 | X55005 | Hs.724 | 7067 | c-erbA-1/ |
| | | | | | THRA |
| 16 | 42 | X72631 | Hs.2769166 | 9572 | NR1D1 |
| 17 | 43 | NM_007359.1 | Hs.83422 | 22794 | MLN51 |
| 18 | 44 | U77949.1 | Hs.405958 | 990 | CDC6 |
| 19 | 45 | U41742.1 | Hs.361071 | 5914 | RARA |
| | | NM_000964 | | | |
| 20 | 46 | NM_001067.1 | Hs.156346 | 7153 | TOP2A |
| 21 | 47 | NM_001552.1 | Hs.1516 | 3487 | IGFBP4 |
| 22 | 48 | NM_001838.1 | Hs.1652 | 1236 | CCR7 EBI1 |
| 23 | 49 | NM_003079.1 | Hs.437546 | 6605 | SMARCE1 BAF57 |
| 24 | 50 | X14487 | Hs.99936 | 3858 | KRT10 |
| 25 | 51 | NM_000223.1 | Hs.66739 | 3859 | KRT12 |
| 26 | 52 | NM_002279.2 | Hs.32950 | 3884 | /KRTHA3B |
| 53 | 76 | NM_005937 | Hs.497128 | 4302 | MLLT6 |
| 54 | 77 | XM_008147/ | Hs.371617 | 7703 | ZNF144/RNF110 |
| | | NM_007144 | | | |
| 55 | 78 | NM_138687 | Hs.9605 | 8396 | PIP5K2B |
| 56 | 79 | NM_020405 | Hs.125036 | 57125 | TEM7/PLXDC1 |
| 57 | 80 | AF129512 | Hs.258579 | 22806 | ZNFN1A3 |
| 58 | 81 | XM_085731 | Hs.421622 | 147179 | WIRE |
| | | NM_133264 | | | |
| 59 | 82 | NM_002795 | Hs.82793 | 5691 | PSMB3 |
| 60 | 83 | NM_033419 | Hs.91668 | 93210 | MGC9753 |
| | | | | | Variant a/ |
| | | | | | CAB2 |
| 61 | 84 | NM_033419 | Hs.91668 | 93210 | MGC9753 |
| | | | | | Variant c |
| 62 | 85 | NM_033419 | Hs.91668 | 93210 | MGC9753 |
| | | | | | Variant d |
| 63 | 86 | NM_033419 | Hs.91668 | 93210 | MGC9753 |
| | | | | | Variant e |
| 64 | 87 | NM_033419 | Hs.91668 | 93210 | MGC9753 |
| | | | | | Variant g |
| 65 | 88 | NM_033419 | Hs.91668 | 93210 | MGC9753 |
| | | | | | Variant h |
| 66 | 89 | NM_033419 | Hs.91668 | 93210 | MGC9753 |
| | | | | | Variant i |
| 67 | 90 | AF395708 | Hs.133167 | 94103 | ORMDL3 |
| 68 | 91 | NM_032875 | Hs.194498 | 84961 | MGC15482 |
| 69 | 92 | NM_032192 | Hs.286192 | 84152 | PPP1R1B |
| 70 | 93 | NM_032339 | Hs.333526 | 84299 | MGC14832 |
| 71 | 94 | NM_057555 | Hs.133167 | 51242 | LOC51242/ |
| | | NM_139280 | | | ORMDL3 |
| 72 | 95 | NM_017748 | Hs.406223 | 54883 | FLJ20291 |
| 73 | 96 | NM_018530 | Hs.306777 | 55876 | Pro2521 |
| 74 | 97 | NM_016339 | Hs.158530 | 51195 | Link-GEFII |
| 75 | 98 | NM_032865 | Hs.99037 | 84951 | CTEN |
| 315 | 393 | XM_294897 | Hs.270564 | 30837 | NAP4 |
| 316 | 394 | NM_032351 | Hs.19347 | 84311 | MRLP45 |
| 317 | 395 | NM_000458 | Hs.408093 | 6928 | TCF2 |
| 318 | 396 | NM_152300 | Hs.380430 | 11056 | ROK1 |
| 319 | 397 | NM_019010 | Hs.84905 | 54474 | KRT20 |
| 320 | 398 | NM_173213 | Hs.9029 | 25984 | KRT23 |
| 321 | 399 | NM_033185 | Hs.307025 | 85293 | KRTAP3-3 |
| 322 | 400 | NM_031959 | Hs.307026 | 83897 | KRTAP3-2 |
| 323 | 401 | NG_000941 | | 85345 | KRTAP3P1 |
| 324 | 402 | NM_031958 | Hs.307027 | 83896 | KRTAP3-1 |
| 325 | 403 | NM_031957 | Hs.307030 | 83895 | KRTAP1-5 |
| 326 | 404 | NM_030966 | Hs.247935 | 81850 | KRTAP1-3 |
| 327 | 405 | NM_030967 | Hs.247934 | 81851 | KRTAP1-1 |
| 328 | 406 | AJ302536 | | 85296 | KRTAP2-2 |
| 329 | 407 | NM_033184 | | 85294 | KRTAP2-4 |
| 330 | 408 | NG_000939 | | 85343 | KRTAP2P1 |
| 331 | 409 | NM_033061 | Hs.380164 | 85287 | KRTAP4-7 |
| 332 | 410 | NM_033059 | Hs.307015 | 85282 | KRTAP4-14 |
| 333 | 411 | NM_031854 | Hs.307016 | 83755 | KRTAP4-12 |
| 334 | 412 | NM_033188 | Hs.307016 | 83755 | KRTAP4-5 |
| 335 | 413 | NM_033186 | | 85283 | KRTAP4-13 |
| 336 | 414 | NM_032524 | Hs.307022 | 84616 | KRTAP4-4 |
| 337 | 415 | NM_033062 | Hs.380165 | 85291 | KRTAP4-2 |
| 338 | 416 | NM_033060 | Hs.380165 | 85291 | KRTAP4-10 |
| 339 | 417 | NM_031961 | Hs.307013 | 83899 | KRTAP9-2 |
| 340 | 418 | NM_031962 | Hs.307012 | 83900 | KRTAP9-3 |
| 341 | 419 | NM_031963 | Hs.307011 | 83901 | KRTAP9-8 |
| 342 | 420 | NM_030975 | Hs.307010 | 81870 | KRTAP9-9 |
| 343 | 421 | NM_033191 | | 85280 | KRTAP9-4 |
| 344 | 422 | NG_000942 | | 85347 | KRTAP9P1 |
| 345 | 423 | XM_210345 | Hs.463016 | 85276 | KRTAP16-1 |
| 346 | 424 | NM_031964 | Hs.307009 | 83902 | KRTAP17-1 |
| 347 | 425 | NM_004138 | Hs.197874 | 3883 | KRTHA3A |
| 348 | 426 | NM_002279 | Hs.32950 | 3884 | KRTHA3B |
| 349 | 427 | NM_021013 | Hs.296942 | 3885 | KRTHA4 |
| 350 | 428 | NM_002277 | Hs.41696 | 3881 | KRTHA1 |
| 351 | 429 | Y16795 | | 8686 | KRTHAP1 |
| 352 | 430 | NM_003770 | Hs.159403 | 8688 | KRTHA7 |
| 353 | 431 | NM_006771 | Hs.248188 | 8687 | KRTHA8 |
| 354 | 432 | NM_002278 | Hs.41752 | 3882 | KRTHA2 |
| 355 | 433 | NM_002280 | Hs.73082 | 3886 | KRTHA5 |
| 356 | 434 | NM_003771 | Hs.248189 | 8689 | KRTHA6 |
| 357 | 435 | NM_002274 | Hs.433871 | 3860 | KRT13 |
| 358 | 436 | NM_002275 | Hs.80342 | 3866 | KRT15 |
| 359 | 437 | NM_002276 | Hs.309517 | 3880 | KRT19 |
| 360 | 438 | NM_000226 | Hs.2783 | 3857 | KRT9 |
| 361 | 439 | NM_000526 | Hs.355214 | 3861 | KRT14 |
| 362 | 440 | NM_005557 | Hs.432448 | 3868 | KRT16 |
| 363 | 441 | NM_000422 | Hs.2785 | 3872 | KRT17 |
| 364 | 442 | NM_005556 | Hs.23881 | 3855 | KRT7 |
| 365 | 443 | NG_000944 | | 85349 | KRTHBP4 |
| 366 | 444 | NG_000943 | | 85348 | KRTHBP3 |
| 367 | 445 | NM_002281 | Hs.170925 | 3887 | KRTHB1 |
| 368 | 446 | NM_002284 | Hs:278658 | 3892 | KRTHB6 |
| 369 | 447 | NM_002282 | Hs.182506 | 3889 | KRTHB3 |
| 370 | 448 | NG_000940 | | 85344 | KRTHBP2 |
| 371 | 449 | NM_002283 | Hs.182507 | 3891 | KRTHB5 |
| 372 | 450 | NM_033045 | Hs.272336 | 3890 | KRTHB4 |
| 373 | 451 | NM_033033 | Hs.134640 | 3888 | KRTHB2 |
| 374 | 452 | Y19213 | | 85340 | KRTHBP1 |
| 375 | 453 | NM_005555 | Hs.432677 | 3854 | KRT6B |
| 376 | 454 | NM_173086 | Hs.446417 | 286887 | KRT6E |
| 377 | 455 | NM_058242 | | 140446 | KRT6C |
| 378 | 456 | NM_005554 | Hs.367762 | 3853 | KRT6A |
| 379 | 457 | NM_000424 | Hs.433845 | 3852 | KRT5 |
| 380 | 458 | NM_033448 | Hs.55278 | 112802 | KRT6IRS |
| 381 | 459 | NM_175053 | Hs.56255 | 121391 | KRT6IRS4 |
| 382 | 460 | NM_080747/ | Hs.147040 | 140807 | K6IRS2/ |
| | | AY033495 | | | KRT6 |
| 383 | 461 | NM_175068 | Hs.319101 | 55410 | KRT6IRS3 |
| 384 | 462 | NM_000423 | Hs.707 | 3849 | KRT2A |
| 385 | 463 | NM_006121 | Hs.80828 | 3848 | KRT1 |
| 386 | 464 | NM_057088 | Hs.410397 | 3850 | KRT3 |
| 387 | 465 | NM_002272 | Hs.371139 | 3851 | KRT4 |
| 388 | 466 | NM_002273 | Hs.356123 | 3856 | KRT8 |
| 389 | 467 | NM_000224 | Hs.406013 | 3875 | KRT18 |
| 390 | 468 | NM_032950 | Hs.380710 | 79148 | MMP28 |
| 391 | 469 | NM_005419 | Hs.72988 | 6773 | STAT2 |
| 392 | 470 | NM_002046 | Hs.169476 | 2597 | GAPDH |

TABLE 2

| DNA | |
|---|---|
| SEQ ID NO: | Gene description |
| 1 | Member of a subfamily of LIM proteins that contains a LIM domain and an |
| | SH3 (Src homology region 3) domain |
| 2 | Beta 1 subunit of a voltage-dependent calcium channel (dihydropyridine |
| | receptor), involved in coupling of excitation and contraction in muscle, also |
| | acts as a calcium channel in various other tissues |
| 3 | Ribosomal protein L19, component of the large 60S ribosomal subunit |
| 4 | Protein with similarity to nuclear receptor-interacting proteins; binds and co- |
| | activates the nuclear receptors PPARalpha (PPARA), RARalpha (RARA), |
| | RXR, TRbeta1, and VDR |
| 5 | we26e02.x1 CDC2-related protein kinase 7 |
| 6 | Neurogenic differentiation, a basic-helix-loop-helix transcription factor that |
| | mediates neuronal differentiation |
| 7 | Protein that is overexpressed in malignant tissues, contains a putative trans- |
| | membrane region and a StAR Homology Domain (SHD), may function in |
| | steroidogenesis and contribute to tumor progression |
| 8 | Telethonin, a sarcomeric protein specifically expressed in skeletal and heart |
| | muscle, caps titin (TTN) and is important for structural integrity of the |
| | sarcomere |
| 9 | Phenylethanolamine N-methyltransferase, acts in catecholamine biosynthesis |
| | to convert norepinephrine to epinephrine |
| 10 | Tyrosine kinase receptor that has similarity to the EGF receptor, a critical |
| | component of IL-6 signaling through the MAP kinase pathway, overexpression |
| | associated with prostate, ovary and breast cancer |
| 11 | Growth factor receptor-bound protein, an SH2 domain-containing protein that |
| | has isoforms which may have a role in cell invasion and metastatic progression |
| | of esophageal carcinomas |
| 12 | Non-ATPase subunit of the 26S proteasome (prosome, macropain) |
| 13 | Granulocyte colony stimulating factor, a glycoprotein that regulates growth, |
| | differentiation, and survival of neutrophilic granulocytes |
| 14 | Member of the Vitamin D Receptor Interacting Protein co-activator complex, |
| | has strong similarity to thyroid hormone receptor-associated protein (murine |
| | Trap100) which function as a transcriptional coregulator |
| 15 | Thyroid hormone receptor alpha, a high affinity receptor for thyroid hormone |
| | that activates transcription; homologous to avian erythroblastic leukemia virus |
| | oncogene |
| 16 | encoding Rev-ErbAalp nuclear receptor subfamily 1, group D, member 1 |
| 17 | Protein that is overexpressed in breast carcinomas |
| 18 | Protein which interacts with the DNA replication proteins PCNA and Orc1, |
| | translocates from the nucleus following onset of S phase; S. cerevisiae |
| | homolog Cdc6p is required for initiation of S phase |
| 19 | Retinoic acid receptor alpha, binds retinoic acid and stimulates transcription in |
| | a ligand-dependent manner |
| 20 | DNA topoisomerase II alpha, member of a family of proteins that relieves |
| | torsional stress created by DNA replication, transcription, and cell division; |
| 21 | Insulin-like growth factor binding protein, the major IGFBP of osteoblast-like |
| | cells, binds IGF1 and IGF2 and inhibits their effects on promoting DNA and |
| | glycogen synthesis in osteoblastic cells |
| 22 | HUMEBI103 G protein-coupled receptor (EBI 1) gene exon 3 chemokine (C-C |
| | motif) receptor 7 G protein-coupled receptor |
| 23 | Protein with an HMG 1/2 DNA-binding domain that is subunit of the |
| | SNF/SWI complex associated with the nuclear matrix and implicated in |
| | regulation of transcription by affecting chromatin structure |
| 24 | Keratin 10, a type I keratin that is a component of intermediate filaments and is |
| | expressed in terminally differentiated epidermal cells; mutation of the |
| | corresponding gene causes epidermolytic hyperkeratosis |
| 25 | Keratin 12, a component of intermediate filaments in corneal epithelial cells; |
| | mutation of the corresponding gene causes Meesmann corneal dystrophy |
| 26 | Hair keratin 3B, a type I keratin that is a member of a family of structural |
| | proteins that form intermediate filaments |
| 53 | MLLT6 Myeloid/lymphoid or mixed-lineage leukemia (trithorax homolog, |
| | Drosophila); translocated to, 6 |
| 54 | zinc finger protein 144 (Mel-18) |
| 55 | Phosphatidylinositol-4-phosphate 5-kinase type II beta isoform a |
| 56 | tumor endothelial marker 7 precursor |
| 57 | zinc finger protein, subfamily 1A, 3 |
| 58 | WASP-binding protein putative cr16 and wip like protein similar to Wiskott- |
| | Aldrich syndrome protein |
| 59 | Proteasome (prosome, macropain) subunit, beta type, 3 |
| 60 | Predicted |
| 67 | ORM1-like 3 (S. cerevisiae) |
| 68 | F-box domain A Receptor for Ubiquitination Targets |
| 69 | protein phosphatase 1, regulatory (inhibitor) subunit 1B (dopamine and cAMP |
| | regulated phosphoprotein, DARPP-32) |
| 70 | Predicted Protein |
| 71 | Predicted Protein |
| 72 | Predicted Protein |
| 73 | Predicted Protein |
| 74 | Link-GEFII: Link guanine nucleotide exchange factor II |
| 75 | C-terminal tensin-like |
| 315 | Homo sapiens Nck, Ash and phospholipase C binding protein (NAP4) |
| 316 | Homo sapiens mitochondrial ribosomal protein L45 (MRPL45), nuclear gene |
| | encoding mitochondrial protein |
| 317 | Homo sapiens transcription factor 2, hepatic; LF-B3; variant hepatic nuclear |
| | factor (TCF2), transcript variant a |
| 318 | Homo sapiens DEAD (Asp-Glu-Ala-Asp) box polypeptide 52 (DDX52) |
| 319 | Homo sapiens keratin 20 (KRT20),, is a component of intermediate filament |
| | network |
| 320 | Homo sapiens keratin 23 (histone deacetylase inducible) (KRT23), is a |
| | component of intermediate filament network transcript variant 2 |
| 321 | Homo sapiens keratin associated protein 3-3 (KRTAP3-3)), is a component of |
| | intermediate filament network |
| 322 | Homo sapiens keratin associated protein 3-2 (KRTAP3-2), is a component of |
| | intermediate filament network |
| 323 | Homo sapiens keratin associated protein 3 pseudogene 1 (KRTAP3P1) on |
| | chromosome 17, is a component of intermediate filament network |
| 324 | Homo sapiens keratin associated protein 3-1 (KRTAP3-1), is a component of |
| | intermediate filament network |
| 325 | Homo sapiens keratin associated protein 1-5 (KRTAP1-5), is a component of |
| | intermediate filament network |
| 326 | Homo sapiens keratin associated protein 1-3 (KRTAP1-3), is a component of |
| | intermediate filament network |
| 327 | Homo sapiens keratin associated protein 1-1 (KRTAP1-1), is a component of |
| | intermediate filament network |
| 328 | HSA302536 Homo sapiens partial mRNA for keratin associated protein |
| | KAP2.2 (KRTAP2.2 gene), is a component of intermediate filament network |
| 329 | Homo sapiens keratin associated protein 2-4 (KRTAP2-4), is a component of |
| | intermediate filament network |
| 330 | Homo sapiens keratin associated protein 2 pseudogene 1 (KRTAP2P1) on |
| | chromosome 17, is a component of intermediate filament network |
| 331 | Homo sapiens keratin associated protein 4-7 (KRTAP4-7), is a component of |
| | intermediate filament network |
| 332 | Homo sapiens keratin associated protein 4-14 (KRTAP4-14), is a component |
| | of intermediate filament network |
| 333 | Homo sapiens keratin associated protein 4-12 (KRTAP4-12), is a component |
| | of intermediate filament network |
| 334 | Homo sapiens keratin associated protein 4-5 (KRTAP4-5), is a component of |
| | intermediate filament network |
| 335 | Homo sapiens keratin associated protein 4-13 (KRTAP4-13), is a component |
| | of intermediate filament network |
| 336 | Homo sapiens keratin associated protein 4-4 (KRTAP4-4), is a component of |
| | intermediate filament network |
| 337 | Homo sapiens keratin associated protein 4-2 (KRTAP4-2), is a component of |
| | intermediate filament network |
| 338 | Homo sapiens keratin associated protein 4-10 (KRTAP4-10), is a component |
| | of intermediate filament network |
| 339 | Homo sapiens keratin associated protein 9-2 (KRTAP9-2), is a component of |
| | intermediate filament network |
| 340 | Homo sapiens keratin associated protein 9-3 (KRTAP9-3), is a component of |
| | intermediate filament network |
| 341 | Homo sapiens keratin associated protein 9-8 (KRTAP9-8), is a component of |
| | intermediate filament network |
| 342 | Homo sapiens keratin associated protein 9-9 (KRTAP9-9), is a component of |
| | intermediate filament network |
| 343 | Homo sapiens keratin associated protein 9-4 (KRTAP9-4), is a component of |
| | intermediate filament network |
| 344 | Homo sapiens keratin associated protein 9 pseudogene 1 (KRTAP9P1) on |
| | chromosome 17, is a component of intermediate filament network |
| 345 | Homo sapiens keratin associated protein 16-1 (KRTAP16-1), is a component |
| | of intermediate filament network |
| 346 | Homo sapiens keratin associated protein 17-1 (KRTAP17-1), is a component |
| | of intermediate filament network |
| 347 | Homo sapiens keratin, hair, acidic, 3A (KRTHA3A), is a component of |
| | intermediate filament network |
| 348 | Homo sapiens keratin, hair, acidic, 3B (KRTHA3B), is a component of |
| | intermediate filament network |
| 349 | Homo sapiens keratin, hair, acidic, 4 (KRTHA4), is a component of |
| | intermediate filament network |
| 350 | Homo sapiens keratin, hair, acidic, 1 (KRTHA1), is a component of |
| | intermediate filament network |
| 351 | HSA16795 Homo sapiens KRTHAP1 pseudogene, is a component of |
| | intermediate filament network |
| 352 | Homo sapiens keratin, hair, acidic, 7 (KRTHA7), is a component of |
| | intermediate filament network |
| 353 | Homo sapiens keratin, hair, acidic, 8 (KRTHA8), is a component of |
| | intermediate filament network |
| 354 | Homo sapiens keratin, hair, acidic, 2 (KRTHA2), is a component of |
| | intermediate filament network |
| 355 | Homo sapiens keratin, hair, acidic, 5 (KRTHA5), is a component of |
| | intermediate filament network |
| 356 | Homo sapiens keratin, hair, acidic, 6 (KRTHA6), is a component of |
| | intermediate filament network |
| 357 | Homo sapiens keratin 13 (KRT13), transcript variant 2, is a component of |
| | intermediate filament network |
| 358 | Homo sapiens keratin 15 (KRT15), is a component of intermediate filament |
| | network |
| 359 | Homo sapiens keratin 19 (KRT19), is a component of intermediate filament |
| | network |
| 360 | Homo sapiens keratin 9 (epidermolytic palmoplantar keratoderma) (KRT9), is |
| | a component of intermediate filament network |
| 361 | Homo sapiens keratin 14 (epidermolysis bullosa simplex, Dowling-Meara, |
| | Koebner) (KRT14), is a component of intermediate filament network |
| 362 | Homo sapiens keratin 16 (focal non-epidermolytic palmoplantar keratoderma) |
| | (KRT16), is a component of intermediate filament network |
| 363 | Homo sapiens keratin 17 (KRT17), is a component of intermediate filament |
| | network |
| 364 | Homo sapiens keratin 7 (KRT7), is a component of intermediate filament |
| | network |
| 365 | Homo sapiens psihHbD hair keratin pseudogene (KRTHBP4) on chromosome |
| | 12, is a component of intermediate filament network |
| 366 | Homo sapiens psihHbC hair keratin pseudogene (KRTHBP3) on chromosome |
| | 12, is a component of intermediate filament network |
| 367 | Homo sapiens keratin, hair, basic, 1 (KRTHB1), is a component of |
| | intermediate filament network |
| 368 | Homo sapiens keratin, hair, basic, 6 (monilethrix) (KRTHB6), is a component |
| | of intermediate filament network |
| 369 | Homo sapiens keratin, hair, basic, 3 (KRTHB3), is a component of |
| | intermediate filament network |
| 370 | Homo sapiens psihHbB hair keratin pseudogene (KRTHBP2) on chromosome |
| | 12, is a component of intermediate filament network |
| 371 | Homo sapiens keratin, hair, basic, 5 (KRTHB5), is a component of |
| | intermediate filament network |
| 372 | Homo sapiens keratin, hair, basic, 4 (KRTHB4),, is a component of |
| | intermediate filament network |
| 373 | Homo sapiens keratin, hair, basic, 2 (KRTHB2), is a component of |
| | intermediate filament network |
| 374 | HSPSIHHBA Homo sapiens putative psihHbA pseudogene for hair keratin, |
| | exons 2 to 7 |
| 375 | Homo sapiens keratin 6B (KRT6B), is a component of intermediate filament |
| | network |
| 376 | Homo sapiens keratin 6E (KRT6E), is a component of intermediate filament |
| | network |
| 377 | Homo sapiens keratin 6C (KRT6C), is a component of intermediate filament |
| | network |
| 378 | Homo sapiens keratin 6A (KRT6A),, is a component of intermediate filament |
| | network |
| 379 | Homo sapiens keratin 5 (epidermolysis bullosa simplex, Dowling- |
| | Meara/Kobner/Weber-Cockayne types) (KRT5), is a component of |
| | intermediate filament network |
| 380 | Homo sapiens keratin 6 irs (KRT6IRS), is a component of intermediate |
| | filament network |
| 381 | Homo sapiens keratin 6 irs4 (K6IRS4), is a component of intermediate |
| | filament network |
| 382 | Homo sapiens keratin protein K6irs (K6IRS2), is a component of intermediate |
| | filament network |
| 383 | Homo sapiens keratin protein K6irs (K6IRS2), is a component of intermediate |
| | filament network |
| 384 | Homo sapiens keratin 2A (epidermal ichthyosis bullosa of Siemens) (KRT2A), |
| | is a component of intermediate filament network |
| 385 | Homo sapiens keratin 1 (epidermolytic hyperkeratosis) (KRT1), is a |
| | component of intermediate filament network |
| 386 | Homo sapiens keratin 3 (KRT3), is a component of intermediate filament |
| | network |
| 387 | Homo sapiens keratin 4 (KRT4), is a component of intermediate filament |
| | network |
| 388 | Homo sapiens keratin 8 (KRT8), is a component of intermediate filament |
| | network |
| 389 | Homo sapiens keratin 18 (KRT18), is a component of intermediate filament |
| | network |
| 390 | Homo sapiens matrix metalloproteinase 28 (MMP28), transcript variant 2 |
| 391 | Homo sapiens signal transducer and activator of transcription 2, 113 kDa |
| | (STAT2) |
| 392 | Homo sapiens glyceraldehyde-3-phosphate dehydrogenase (GAPD) |

TABLE 3

| DNA | | |
|---|---|---|
| SEQ ID | | |
| NO: | Gene function | Subcellular localization |
| 1 | SH3/SH2 adapter protein | — |
| 2 | voltage-gated calcium channel membrane fraction Channel [passive transporter] | Plasma membrane |
| 3 | RNA binding structural protein of ribosome protein biosynthesis | Cytoplasm |
| 4 | transcription co-activator nucleus Pol II transcription | Nucleus |
| 5 | — | — |
| 6 | transcription factor transcription regulation from Pol II promoter neurogenesis | — |
| 7 | mitochondrial transport steroid and lipid metabolism | Cytoplasm |
| 8 | structural protein of muscle sarcomere alignment | Cytoplasm |
| 9 | phenylethanolamine N-methyltransferase Transferase | — |
| 10 | Neu/ErbB-2 receptor receptor signaling protein tyrosine kinase | Plasma membrane |
| 11 | SH3/SH2 adapter protein EGF receptor signaling pathway | Cytoplasm |
| 12 | 26S proteasome Protein degradation Proteasome subunit | Cytoplasm |
| 13 | developmental processes positive control of cell proliferation | Extracellular space |
| 14 | fatty acid omega-hydroxylase fatty acid omega-hydroxylase | — |
| 15 | DNA-binding protein Transcription factor | Nucleus |
| 16 | steroid hormone receptor transcription co-repressor | Nucleus |
| 17 | — | — |
| 18 | nucleotide binding cell cycle regulator DNA replication checkpoint regulation of CDK activity | nucleus |
| 19 | retinoic acid receptor transcription co-activator transcription factor | nucleus |
| 20 | DNA binding DNA topoisomerase (ATP-hydrolyzing) | nucleus |
| 21 | skeletal development DNA metabolism signal transduction cell proliferation | |
| 22 | | plasma membrane |
| 23 | chromatin binding transcription co-activator nucleosome disassembly transcription | nucleus nuclear chromosome |
| 24 | Cell structure Cytoskeletal Epidermal Development and Maintenance | cytoplasm |
| 25 | structural protein vision cell shape and cell size control intermediate filament | cytoplasm |
| 26 | cell shape and cell size control Cell structure | cytoplasm |
| 53 | | — |
| 54 | leucine-zipper containing fusion | — |
| 55 | | |
| 56 | Tumor endothelial marker 7 precursor; may be involved in angiogenesis | — |
| 57 | Aiolos; DNA binding protein that may be a transcription factor; has strong similarity to murine | — |
| | Znfn1a3, contains zinc finger domain | |
| 58 | The WASP-binding protein WIRE has a role in the regulation of the actin filament system | — |
| | downstream of the platelet-derived growth factor receptor | |
| 59 | | — |
| 60 | | — |
| 61 | | — |
| 67 | | — |
| 68 | | — |
| 69 | Midbrain dopaminergic neurons play a critical role in multiple brain functions, and abnormal | — |
| | signaling through dopaminergic pathways has been implicated in several major neurologic | |
| | and psychiatric disorders. One well-studied target for the actions of dopamine is DARPP32. | |
| 70 | | — |
| 71 | | — |
| 72 | | — |
| 73 | | — |
| 74 | Brain-specific guanine nucleotide exchange factor; activates the ERK/MAP kinase cascade plus | — |
| | R-Ras and H-ras; activates targets through a Ca2+- and diacylglycerol-sensitive mechanism; | |
| | active protein associates with membranes | |
| 75 | C-terminal tensin-like Phosphotyrosine-binding domain, phosphotyrosine-interaction (PI) domain | — |
| 315 | | |
| 316 | | |
| 317 | | |
| 318 | | cytoplasm |
| 319 | KRT20, integral part of the intermediate filamentous network | Cytoplasm |
| 320 | KRT23, integral part of the intermediate filamentous network | Cytoplasm |
| 321 | KRTAP3-3, integral part of the intermediate filamentous network | Cytoplasm |
| 322 | KRTAP3-2, integral part of the intermediate filamentous network | Cytoplasm |
| 323 | KRTAP3P1, integral part of the intermediate filamentous network | Cytoplasm |
| 324 | KRTAP3-1, integral part of the intermediate filamentous network | Cytoplasm |
| 325 | KRTAP1-5, integral part of the intermediate filamentous network | cytoplasm |
| 326 | KRTAP1-3, integral part of the intermediate filamentous network | cytoplasm |
| 327 | KRTAP1-1, integral part of the intermediate filamentous network | Cytoplasm |
| 328 | KRTAP2-2, integral part of the intermediate filamentous network | Cytoplasm |
| 329 | KRTAP2-4, integral part of the intermediate filamentous network | Cytoplasm |
| 330 | KRTAP2P1, integral part of the intermediate filamentous network | Cytoplasm |
| 331 | KRTAP4-7, integral part of the intermediate filamentous network | Cytoplasm |
| 332 | KRTAP4-14, integral part of the intermediate filamentous network | Cytoplasm |
| 333 | KRTAP4-12, integral part of the intermediate filamentous network | cytoplasm |
| 334 | KRTAP4-5, integral part of the intermediate filamentous network | cytoplasm |
| 335 | KRTAP4-13, integral part of the intermediate filamentous network | Cytoplasm |
| 336 | KRTAP4-4, integral part of the intermediate filamentous network | Cytoplasm |
| 337 | KRTAP4-2, integral part of the intermediate filamentous network | Cytoplasm |
| 338 | KRTAP4-10, integral part of the intermediate filamentous network | Cytoplasm |
| 339 | KRTAP9-2, integral part of the intermediate filamentous network | Cytoplasm |
| 340 | KRTAP9-3, integral part of the intermediate filamentous network | Cytoplasm |
| 341 | KRTAP9-8, integral part of the intermediate filamentous network | cytoplasm |
| 342 | KRTAP9-9, integral part of the intermediate filamentous network | cytoplasm |
| 343 | KRTAP9-4, integral part of the intermediate filamentous network | Cytoplasm |
| 344 | KRTAP9P1, integral part of the intermediate filamentous network | Cytoplasm |
| 345 | KRTAP16-1, integral part of the intermediate filamentous network | Cytoplasm |
| 346 | KRTAP17-1, integral part of the intermediate filamentous network | Cytoplasm |
| 347 | KRTHA3A, integral part of the intermediate filamentous network | Cytoplasm |
| 348 | KRTHA3B, integral part of the intermediate filamentous network | Cytoplasm |
| 349 | KRTHA4, integral part of the intermediate filamentous network | cytoplasm |
| 350 | KRTHA1, integral part of the intermediate filamentous network | cytoplasm |
| 351 | KRTHAP1, integral part of the intermediate filamentous network | Cytoplasm |
| 352 | KRTHA7, integral part of the intermediate filamentous network | Cytoplasm |
| 353 | KRTHA8, integral part of the intermediate filamentous network | Cytoplasm |
| 354 | KRTHA2, integral part of the intermediate filamentous network | Cytoplasm |
| 355 | KRTHA5, integral part of the intermediate filamentous network | Cytoplasm |
| 356 | KRTHA6, integral part of the intermediate filamentous network | Cytoplasm |
| 357 | KRT13, integral part of the intermediate filamentous network | cytoplasm |
| 358 | KRT15, integral part of the intermediate filamentous network | cytoplasm |
| 359 | KRT19, integral part of the intermediate filamentous network | Cytoplasm |
| 360 | KRT9, integral part of the intermediate filamentous network | Cytoplasm |
| 361 | KRT14, integral part of the intermediate filamentous network | Cytoplasm |
| 362 | KRT16, integral part of the intermediate filamentous network | Cytoplasm |
| 363 | KRT17, integral part of the intermediate filamentous network | Cytoplasm |
| 364 | KRT7, integral part of the intermediate filamentous network | Cytoplasm |
| 365 | KRTHBP4, integral part of the intermediate filamentous network | cytoplasm |
| 366 | KRTHBP3, integral part of the intermediate filamentous network | cytoplasm |
| 367 | KRTHB1, integral part of the intermediate filamentous network | Cytoplasm |
| 368 | KRTHB6, integral part of the intermediate filamentous network | Cytoplasm |
| 369 | KRTHB3, integral part of the intermediate filamentous network | Cytoplasm |
| 370 | KRTHBP2, integral part of the intermediate filamentous network | Cytoplasm |
| 371 | KRTHB5, integral part of the intermediate filamentous network | Cytoplasm |
| 372 | KRTHB4, integral part of the intermediate filamentous network | Cytoplasm |
| 373 | KRTHB2, integral part of the intermediate filamentous network | cytoplasm |
| 374 | KRTHBP1, integral part of the intermediate filamentous network | cytoplasm |
| 375 | KRT6B, integral part of the intermediate filamentous network | Cytoplasm |
| 376 | KRT6E, integral part of the intermediate filamentous network | Cytoplasm |
| 377 | KRT6C, integral part of the intermediate filamentous network | Cytoplasm |
| 378 | KRT6A, integral part of the intermediate filamentous network | Cytoplasm |
| 379 | KRT5, integral part of the intermediate filamentous network | Cytoplasm |
| 380 | KRT6IRS, integral part of the intermediate filamentous network | Cytoplasm |
| 381 | KRT6IRS4, integral part of the intermediate filamentous network | cytoplasm |
| 382 | KRT6, integral part of the intermediate filamentous network | Cytoplasm |
| 383 | KRT6IRS3, integral part of the intermediate filamentous network | Cytoplasm |
| 384 | KRT2A, integral part of the intermediate filamentous network | Cytoplasm |
| 385 | KRT1, integral part of the intermediate filamentous network | Cytoplasm |
| 386 | KRT3, integral part of the intermediate filamentous network | Cytoplasm |
| 387 | KRT4, integral part of the intermediate filamentous network | Cytoplasm |
| 388 | KRT8, integral part of the intermediate filamentous network | cytoplasm |
| 389 | KRT18, integral part of the intermediate filamentous network | Cytoplasm |

TABLE 4

| | | | | | | |
|---|---|---|---|---|---|---|
| | Pro- | | | | | |
| DNA | tein | | | | | |
| SEQ | SEQ | | | | | |
| ID | ID | Gene | | | | |
| NO: | NO: | Name | DBSNP ID | Type | Codon | AA-Seq |

| | | | | | | |
|---|---|---|---|---|---|---|
| 9 | 34 | ERBB2 | rs2230698 | coding- | TCA\|TCG | S\|S |
| | | | | synon | | |
| 9 | 34 | ERBB2 | rs2230700 | noncod- | | |
| | | | | ing | | |
| 9 | 34 | ERBB2 | rs1058808 | coding- | CCC\|GCC | P\|A |
| | | | | nonsynon | | |
| 9 | 34 | ERBB2 | rs1801200 | noncod- | | |
| | | | | ing | | |
| 9 | 34 | ERBB2 | rs903506 | noncod- | | |
| | | | | ing | | |
| 9 | 34 | ERBB2 | rs2313170 | noncod- | | |
| | | | | ing | | |
| 9 | 34 | ERBB2 | rs1136201 | coding- | ATC\|GTC | I\|V |
| | | | | nonsynon | | |
| 9 | 34 | ERBB2 | rs2934968 | noncod- | | |
| | | | | ing | | |
| 9 | 34 | ERBB2 | rs2172826 | noncod- | | |
| | | | | ing | | |
| 9 | 34 | ERBB2 | rs1810132 | coding- | ATC\|GTC | I\|V |
| | | | | nonsynon | | |
| 9 | 34 | ERBB2 | rs1801201 | noncod- | | |
| | | | | ing | | |
| 14 | 39 | c- | rs2230702 | coding- | TCC\|TCT | S\|S |
| | | erbA-1 | | synon | | |
| 14 | 39 | c- | rs2230701 | coding- | GCC\|GCT | A\|A |
| | | erbA-1 | | synon | | |
| 14 | 39 | c- | rs1126503 | coding- | ACC\|AGC | T\|S |
| | | erbA-1 | | nonsynon | | |
| 14 | 39 | c- | rs3471 | noncod- | | |
| | | erbA-1 | | ing | | |
| 19 | 44 | TOP2A | rs13695 | noncod- | | |
| | | | | ing | | |
| 19 | 44 | TOP2A | rs471692 | noncod- | | |
| | | | | ing | | |
| 19 | 44 | TOP2A | rs558068 | noncod- | | |
| | | | | ing | | |
| 19 | 44 | TOP2A | rs1064288 | noncod- | | |
| | | | | ing | | |
| 19 | 44 | TOP2A | rs1061692 | coding- | GGA\|GGG | G\|G |
| | | | | synon | | |
| 19 | 44 | TOP2A | rs520630 | noncod- | | |
| | | | | ing | | |
| 19 | 44 | TOP2A | rs782774 | coding- | AAT\|ATT\|A | N\|I\|I\|F |
| | | | | nonsynon | TT\|TTT | |
| 19 | 44 | TOP2A | rs565121 | noncod- | | |
| | | | | ing | | |
| 19 | 44 | TOP2A | rs2586112 | noncod- | | |
| | | | | ing | | |
| 19 | 44 | TOP2A | rs532299 | coding- | TTT\|GTT | F\|V |
| | | | | nonsynon | | |
| 19 | 44 | TOP2A | rs2732786 | noncod- | | |
| | | | | ing | | |
| 19 | 44 | TOP2A | rs1804539 | noncod- | | |
| | | | | ing | | |
| 19 | 44 | TOP2A | rs1804538 | noncod- | | |
| | | | | ing | | |
| 19 | 44 | TOP2A | rs1804537 | noncod- | | |
| | | | | ing | | |
| 19 | 44 | TOP2A | rs1141364 | coding- | AAA\|AAG | K\|K |
| | | | | synon | | |
| 23 | 48 | KRT10 | rs12231 | noncod- | | |
| | | | | ing | | |
| 23 | 48 | KRT10 | rs1132259 | coding- | CAT\|CGT | H\|R |
| | | | | nonsynon | | |
| 23 | 48 | KRT10 | rs1132257 | coding- | CTG\|TTG | L\|L |
| | | | | synon | | |
| 23 | 48 | KRT10 | rs1132256 | coding- | GCC\|GCT | A\|A |
| | | | | synon | | |
| 23 | 48 | KRT10 | rs1132255 | coding- | CTG\|TTG | L\|L |
| | | | | synon | | |
| 23 | 48 | KRT10 | rs1132254 | coding- | GGC\|GGT | G\|G |
| | | | | synon | | |
| 23 | 48 | KRT10 | rs1132252 | coding- | TTC\|TTT | F\|F |
| | | | | synon | | |
| 23 | 48 | KRT10 | rs1132268 | coding- | CAG\|GAG | Q\|E |
| | | | | nonsynon | | |
| 23 | 48 | KRT10 | rs1132258 | coding- | CGG\|TGG | R\|W |
| | | | | nonsynon | | |

TABLE 5

| | |
|---|---|
| PRIMER | SEQUENCE |

| | |
|---|---|
| CACNB1 | FAM 5′ CCATATATAAAACCACTGTCCTGTCCTTTGTGGCT |
| | 3′TAMRA |
| CACNB1 | 5′ CCCCCATCTGTCTGTCTATATTTGTC 3′ |
| FOR | |
| CACNB1 | 5′ TGCCTACGCTGACGACTATGTG 3′ |
| REV | |
| CDC6 | FAM 5′ TTTGGTTTTCTACAACTGTTGCTAT 3′TAMRA |
| CDC6 FOR | 5′ GGGCTCCACACACCAGATG 3′ |
| CDC6 REV | 5′ ACGCTCTGAGCACCCTCTACA 3′ |
| EBI1-1 | FAM 5′ TGTCACAGGGACTGAAAACCTCTCCTCATGT |
| | 3′TAMRA |
| EBI1-1 | 5′ CCCAAGGCCACGAGCTT 3′ |
| FOR | |
| EBI1-1 | 5′ TGTTGCTCTCTTAACGAATCGAAA 3′ |
| REV | |
| EBI1-2 | FAM 5′ CTGGTCAAACAAACTCTCTGAACCCCTCC 3′TAMRA |
| EBI1-2 | 5′ TGGTGAGGAAAAGCGGACAT 3′ |
| FOR | |
| EBI1-2 | 5′ CTGGCTTGGAGGACAGTGAAG 3′ |
| REV | |
| GCSF | FAM 5′ CCAAGCCCTCCCCATCCCATGTAT 3′TAMRA |
| GCSF FOR | 5′ GAGGTGTCGTACCGCGTTCTA 3′ |
| GCSF REV | 5′ CCGTTCTGCTCTTCCCTGTCT 3′ |
| GRB7 | FAM 5′ CCAGACCCGCTTCACTGACCTGC 3′TAMRA |
| GRB7 FOR | 5′ CGCCTGTACTTCAGCATGGA 3′ |
| GRB7 REV | 5′ GCGGTTCAGCTGGTGGAA 3′ |
| HKA3 | FAM 5′ ACCCCGAGGCATCACCACAAATCAT 3′TAMRA |
| HKA3 FOR | 5′ AGTTCTGCCTCTCTGACAACCAT 3′ |
| HKA3 REV | 5′ TAGGCTCAGAGTCAGACCCAAAC 3′ |
| MLN50 | FAM 5′ CCCTCGTGGGCTTGTGCTCGG 3′TAMRA |
| MLN50 | 5′ AAGCCGCCAGTTCATCTTTTT 3′ |
| FOR | |
| MLN50 | 5′ CTTGTGGTTCAAGTCAAATGTTCAG 3′ |
| REV | |
| MLN64-1 | FAM 5′ TCTGCCTGCGCTCTCGTCGGT 3′TAMRA |
| MLN64-1 | 5′ GGGCTGGGCACCTGACTT 3′ |
| FOR | |
| MLN64-1 | 5′ CCCAACAAGGGTCCCAGACT 3′ |
| REV | |
| MLN64-2 | FAM 5′ CGGCGCATTGAGCGGCG 3′TAMRA |
| MLN64-2 | 5′ CCCAAGGGACTTCGTGAATG 3′ |
| FOR | |
| MLN64-2 | 5′ GGCGATCCCTGATGACAAGTA 3′ |
| REV | |
| PPARBP | FAM 5′ AGCACCAAGTGTGAACCAGGTACAATGGC |
| | 3′TAMRA |
| PPARBP | 5′ GAGGGAGGCTCTGCTTTGG 3′ |
| FOR | |
| PPARBP | 5′ TCACAACTAGGGGGTGAGGAG 3′ |
| REV | |
| PSMD3 | FAM 5′ TGCAGAGGAACGGCGTGAGCG 3′TAMRA |
| PSMD3 | 5′ TGAGGTTTCCTCCCAAATCGTA 3′ |
| FOR | |
| PSMD3 | 5′ CAGCTCAAGGGAAGCTGTCATC 3′ |
| REV | |
| RAR | FAM 5′ CCCCCACATGTTCCCCAAGATGCT 3′TAMRA |
| RAR FOR | 5′ GGAGGCGCTAAAGGTCTACGT 3′ |
| RAR REV | 5′ TGATGCTTCGCAGGTCAGTAA 3′ |
| RPL23A | FAM 5′ CTCCTGCCCCTGCTAAAGGTGAAGCC 3′TAMRA |
| RPL23A | 5′ GGACGCGTGGGCTTTTC 3′ |
| FOR | |
| RPL23A | 5′ TGTGGCTGTGGACACCTTTC 3′ |
| REV | |
| RPL19 | FAM 5′ CCACAAGCTGAAGGCAGACAAGGCC 3′TAMRA |
| RPL19 | 5′ GCGGATTCTCATGGAACACA 3′ |
| FOR | |
| RPL19 | 5′ GGTCAGCCAGGAGCTTCTTG 3′ |
| REV | |
| NEUROD2 | FAM 5′ ACCACCTTGCGCAGGTTGTCCAG 3′TAMRA |
| NBUROD2 | 5′ CGCATGCACGACCTGAAC 3′ |
| FOR | |
| NEUROD2 | 5′ GTCTCGATCTTGGACAGCTTCTG 3′ |
| REV | |
| TELE | FAM 5′ ACAGTGTCCACACGGCCCGAGG 3′TAMRA |
| TELE- | |
| THONIN | |
| TELE | 5′ CTGGGCAGAATGGAAGGATCT 3′ |
| TELE- | |
| THONIN | |
| FOR | |
| TELE | 5′ GGGACTCTAGCAGACCCACACT 3′ |
| TELE- | |
| THONIN | |
| REV | |
| PENT | FAM 5′ CACCCACCTGGATTCCCTGTTC 3′TAMRA |
| PNMT | |
| PENT | 5′ CCTTCAGACAGGCGTAGATGATG 3′ |
| PNMT FOR | |
| PENT | 5′ GGGTATTATTTCTTTATTAGGTGCCACTT 3′ |
| PNMT REV | |
| HER2/ | FAM 5′ TTCCCTAAGGCTTTCAGTACCCAGGATCTG |
| NEU; | 3′TAMRA |
| ERBB2 | |
| HER2/ | 5′ CCAGCTTGGCCCTTTCCT 3′ |
| NEU; | |
| ERBB FOR | |
| HER2/ | 5′ GAATGGGTCGCTTTTCGTTCCTTAG 3′ |
| NEU; | |
| ERBB REV | |
| KIA0130 | FAM 5′ TCACGGACCTCAGCGTGCCCCT 3′TAMRA |
| KIA0130 | 5′ TGGTGAAGGTGTCAGCCATGT 3′ |
| FOR | |
| KIA0130 | 5′ TCAGAGTGCAGCAATGGCTTT 3′ |
| REV | |
| THRA | FAM 5′ ACCTCCTTCCCCAGCTCGCG 3′TAMRA |
| THRA FOR | 5′ GGCAACATCTTACTTGTCCTTTGA 3′ |
| THRA REV | 5′ CCAAGGAAGGACAGACAACTATTTC 3′ |
| MLN51 | FAM 5′ TCCTCCCTATCCATGGCACTAAACCACTTC |
| | 3′TAMRA |
| MLN51 | 5′ TGGGCAAGGGCTCCTATCT 3′ |
| FOR | |
| MLN51 | 5′ GTTACCCCTGGCAGACGTATG 3′ |
| REV | |
| TOP2A | FAM 5′ TGCCTCTGAGTCTGAATCTCCCAAAGAGAGA |
| | 3′TAMRA |
| TOP2A | 5′ GAGTAGTTATGTGATTATTTCAGCTCTTGAC 3′ |
| FOR | |
| TOP2A | 5′ TCAAATGTTGTCCCCGAGTCT 3′ |
| REV | |
| KRT10 | FAM 5′ CAGAAATTCGGAAGACAGAACTATTGTCATGCC |
| | 3′TAMRA T |
| KRT10 | 5′ GATTAGTAACCCATAGCAGTTGAAGGT 3′ |
| FOR | |
| KRT10 | 5′ ATTTACTGAGGGTGGTGTGAACATAC 3′ |
| REV | |
| K12 | FAM 5′ TGACAGACTCCAAATCACAAGCACAGTCAAC |
| KRT12 | 3′TAMRA |
| K12 | 5′ TGATGGTTTGGAGGAAAGTTTATTT 3′ |
| KRT12 | |
| FOR | |
| K12 | 5′ TTTGGTTGGGTCTTTAGAGGAATC 3′ |
| KRT12 | |
| REV | |
| NR1D1 | FAM 5′ TGCCAACCATGCATCAGGTAGCCC 3′TAMRA |
| NR1D1 | 5′ CAGCTCACCTGGCAACTTCA 3′ |
| FOR | |
| NR1D1 | 5′ CCTGATTTTCCCAGCGATGT 3′ |
| REV | |
| HSERBT1 | FAM 5′ CGCCGCTCCCGGTTCTGCT 3′TAMRA |
| HSERBT | 5′ TGGCCAAGCGTAAGCTGATT 3′ |
| FOR | |
| HSERBT | 5′ GCTGCAGTGATCGGATCATCT 3′ |
| REV | |
| MLLT6 | FAM 5′ CACCATGGAGCCCATCGTGCTG 3′TAMRA |
| MLLT6 | 5′ ATCCCCGAGGTGCAATTTG 3′ |
| FOR | |
| MLLT6 | 5′ AGCGATCATGAGGCACGTACT 3′ |
| REV | |
| ZNF 144 | FAM 5′ CCTGCCAGAGATAGGAGACCCAGACAGCT |
| | 3′TAMRA |
| ZNF144 | 5′ ATCCCCCTGAGCCTTTTCA 3′ |
| FOR | |
| ZNF144 | 5′ CAGCCTCTGGTCCCACCAT 3′ |
| REV | |
| PIP5K2B | FAM 5′ TGATCATCAATTCCAAACCTCTCCCGAA |
| | 3′TAMRA |
| PIP5K2B | 5′ CCCCATGGTGTTCCGAAAC 3′ |
| FOR | |
| PIP5K2B | 5′ TGCCAGGAGCCTCCATACC 3′ |
| REV | |
| TEM7 | FAM 5′ CAGCCTTCTAAAACACAATGTATTCATGT |
| | 3′TAMRA |
| TEM7 FOR | 5′ CCTGAACTTAATGGTAGAATTCAAAGATC 3′ |
| TEM7 REV | 5′ TATTAACACTGAGAATCCATGCAGAGA 3′ |
| ZNFN1A3 | FAM 5′ TATCTGGTCTCAGGGATTGCTCCTATGTATTCAG |
| | 3′TAMRA C |
| ZNFN1A3 | 5′ CACAGAGCCCTGCTGAAGTG 3′ |
| FOR | |
| ZNFN1A3 | 5′ GCGAGGTCATTGGTTTTTAGAAA 3′ |
| REV | |
| WIRE | FAM 5′ CTGTGATCCGAAATGGTGCCAG 3′TAMRA |
| WIRE FOR | 5′ CCGTCTCCACATCCAAACCT 3′ |
| WIRE REV | 5′ ACCCATGCATTCGGTATGGT 3′ |
| PSMB3 | FAM 5′ AGTGGCACCTGCGCCGAACAA 3′TAMRA |
| PSMB3 | 5′ CCCCATGGTGACTGATGACTT 3′ |
| FOR | |
| PSMB3 | 5′ CCAGAGGGACTCACACATTCC 3′ |
| REV | |
| MGC9753 | FAM 5′ CCAGAAACTTTCCATCCCAAAGGCAGTCT |
| | 3′TAMRA |
| MGC9753 | 5′ CTGCCCCACAGGAATAGAATG 3′ |
| FOR | |
| MGC9753 | 5′ AAAAATCCAGTCTGCTTCAACCA 3′ |
| REV | |
| ORMDL3 | FAM 5′ AGCTGCCCCAGCTCCACGGA 3′TAMRA |
| ORMDL3 | 5′ TCCGTGATGAGCGTGCTTATC 3′ |
| FOR | |
| ORMDL3 | 5′ TCTCAGTACTTATTGATTCCAAAAATCC 3′ |
| REV | |
| MGC15482 | FAM 5′ TCCAGTGGAAGCAACCCCAGTGTTC 3′TAMRA |
| MGC15482 | 5′ CACTTCTAGAGCTACCGTGGAGTCT 3′ |
| FOR | |
| MGC15482 | 5′ CCCTCACTTTGTAACCCTTGCT 3′ |
| REV | |
| PPP1R1B | FAM 5′ CAGCGTGGCGCAACAACCCA 3′TAMRA |
| PPP1R1B | 5′ GGGATTGTTTCGCCACACATA 3′ |
| FOR | |
| PPP1R1B | 5′ CCGATGTTAAGGCCCATAGC 3′ |
| REV | |
| MGC14832 | FAM 5′ TAAAATGTCCGGCCAACATGAGTTCCC 3′TAMRA |
| MGC14832 | 5′ CGCAGTGCCTGGCAGAT 3′ |
| FOR | |
| MGC14832 | 5′ GACACCCCCTGACCTATGGA 3′ |
| REV | |
| LOC51242 | FAM 5′ CAGTGACGTCTCCCGTTCCCTTGGA 3′TAMRA |
| LOC51242 | 5′ TGGGTCCCTGTGTCCTCTTC 3′ |
| FOR | |
| LOC51242 | 5′ AGGGTCAGGAGGGAGAAAAC 3′ |
| REV | |
| FLJ20291 | FAM 5′ CCAGTGCCCACCCGTTAAAGAGTCAA 3′TAMRA |
| FLJ20291 | 5′ TTGTGGGACAGTCAGTAAGTTTGG 3′ |
| FOR | |
| FLJ20291 | 5′ ACAAGCACTCCCACCGAGAT 3′ |
| REV | |
| PRO2521 | FAM 5′ AGTCTGTCCTCACTGCCATCGCCA 3′TAMRA |
| PRO2521 | 5′ AAGCCTCTGGGTTTTCCCTTT 3′ |
| FOR | |
| PRO2521 | 5′ CCCACTGGTGACAGGATGGT 3′ |
| REV | |
| Link- | FAM 5′ CATCTGACATCTTTCGCGTGGAG 3′TAMRA |
| GEFII | |
| Link- | 5′ CTTTGCACGATGTCTCAACCA 3′ |
| GEFII | |
| FOR | |
| Link- | 5′ TTTCCCGTGGAGCAGGAA 3′ |
| GEFII | |
| REV | |
| CTEN | FAM 5′ CCGCCGCCTAATATGCAACATTAGGG 3′TAMRA |
| CTEN FOR | 5′ CGAGTATTCCAAAGCTGGTATCG 3′ |
| CTEN REV | 5′ ATCACAGAGAGATGGCCCTTATCT 3′ |
| NAP4 | FAM 5′ TCCGCCTCAGTCGCCTTTCG 3′TAMRA |
| NAP4 FOR | 5′ TCGGAAGGGCTCCTTCAAA 3′ |
| NAP4 REV | 5′ CACCGTTGCAGCTCTTGGT 3′ |
| MRLP45 | FAM 5′ CTCCCATTCCCCTCATGCTATAAAAAGAACTAC |
| | 3′TAMRA C |
| MRLP45 | 5′ GGCTGCTGGAAGCTTTGAAG 3′ |
| FOR | |
| MRLP45 | 5′ TGAGCAGGATGGGAGAGAACA 3′ |
| REV | |
| TCF2 | FAM 5′ CAAAAGCTGGCCATGGACGCCT 3′TAMRA |
| TCF2 FOR | 5′ GCAGGAAGGAGGAGGCATTC 3′ |
| TCF2 REV | 5′ CAGGCTGTGAGTCTGGTTGGA 3′ |
| ROK1 | FAM 5′ CAGCTGGCTTCCATTTTCCTGGCCT 3′TAMRA |
| ROK1 FOR | 5′ TGGCAAAACTGGGTTCAGAGA 3′ |
| ROK1 REV | 5′ TCGGACGTTGTGGGATGTG 3′ |
| KRT1 | FAM 5′ CCGCCGCCTAATATGCAACATTAGGG 3′TAMRA |
| KRT1 FOR | 5′ CGAGTATTCCAAAGCTGGTATCG 3′ |
| KRT1 REV | 5′ ATCACAGAGAGATGGCCCTTATCT 3′ |
| KRT5 | FAM 5′ CCGCCGCCTAATATGCAACATTAGGG 3′TAMRA |
| KRT5 FOR | 5′ CGAGTATTCCAAAGCTGGTATCG 3′ |
| KRT5 REV | 5′ ATCACAGAGAGATGGCCCTTATCT 3′ |
| KRT8 | FAM 5′ CCGCCGCCTAATATGCAACATTAGGG 3′TAMRA |
| KRT8 FOR | 5′ CGAGTATTCCAAAGGTGGTATCG 3′ |
| KRT8 REV | 5′ ATCACAGAGAGATGGCCCTTATCT 3′ |
| KRT9 | FAM 5′ CCGCCGCCTAATATGCAAGATTAGGG 3′TAMRA |
| KRT9 FOR | 5′ CGAGTATTCCAAAGGTGGTATCG 3′ |
| KRT9 REV | 5′ ATCACAGAGAGATGGCCCTTATCT 3′ |
| KRT10-2 | FAM 5′ CCGCCGCCTAATATGCAACATTAGGG 3′TAMRA |
| KRT10-2 | 5′ CGAGTATTCCAAAGGTGGTATCG 3′ |
| FOR | |
| KRT10-2 | 5′ ATCACAGAGAGATGGCCCTTATCT 3′ |
| REV | |
| KRT14 | FAM 5′ CCGCCGCCTAATATGCAAGATTAGGG 3′TAMRA |
| KRT14 | 5′ CGAGTATTCCAAAGCTGGTATCG 3′ |
| FOR | |
| KRT14 | 5′ ATCACAGAGAGATGGCCCTTATGT 3′ |
| REV | |
| KRT18 | FAM 5′ CGGCCGCCTAATATGCAACATTAGGG 3′TAMRA |
| KRT18 | 5′ CGAGTATTCGAAAGCTGGTATCG 3′ |
| FOR | |
| KRT18 | 5′ ATCACAGAGAGATGGCCCTTATCT 3′ |
| REV | |
| KRT19 | FAM 5′ CCGCCGCCTAATATGCAACATTAGGG 3′TAMRA |
| KRT19 | 5′ CGAGTATTCCAAAGCTGCTATCG 3′ |
| FOR | |
| KRT19 | 5′ ATCACAGAGAGATGGCCCTTATCT 3′ |
| REV | |
| KRT6a/b | FAM 5′ CCGCCGCCTAATATGCAACATTAGGG 3′TAMRA |
| KRT6a/b | 5′ CGAGTATTCCAAAGCTGGTATCG 3′ |
| FOR | |
| KRT6a/b | 5′ ATCACAGAGAGATGGCCCTTATCT 3′ |
| REV | |
| KRT20 | FAM 5′ TGGCGGGAATCCTATTTATCAGACTCTGTAATT |
| | 3′TAMRA GA |
| KRT20 | 5′ GCAAGAAATCAGCCATAAGAAAGC 3′ |
| FOR | |
| KRT20 | 5′ TTGCAGCTCCTGTGAGTAAAACAT 3′ |
| REV | |

TABLE 6

| | | | | | |
|---|---|---|---|---|---|
| No. | ID | forward | reverse | PCR size (bp) | GB ID |

| | | | | | |
|---|---|---|---|---|---|
| 1 | D17S946 | ACAGTCTATCAAGCAGAAAAATCGT | TGCCGTGCCAGAGAGA | 128-142 | Z24029 |
| 2 | D17S1181 | GACAACAGAGCGAGACTCCC | GCCCAGCCTGTCACTTATTC | 122 | — |
| 3 | D17S2026 | TGGTCATTCGACAACGAA | CAGCATTGGATGCAATCC | 171-318 | G05498 X53777 |
| 4 | D17S838 | CTCCAGAATCCAGACCATGA | AGGACAGTGTGTAGCCCTTC | 71-103 | Z51080 |
| 5 | D17S250 | GGAAGAATCAAATAGACAAT | GCTGGCGATATATATATTTAAACC | 151 | — |
| 6 | D17S1818 | CATAGGTATGTTCAGAAATGTGA | TGCCTACTGGAAACCAGA | 119-151 | Z52895 |
| 7 | D17S614 | AAGGGGAAGGGGCTTTCAAAGCT | NGGAGGTTGCAGTGAGCCAAGAT | 136 | L29873 |
| 8 | D17S2019 | CAAAAGCTTATGATGCTCAAACC | TTGTTTCCCTTTGACTTTCTGA | 151-152 | G07286 Z39013 |
| 9 | D17S608 | TAGGTTCACCTCTCATTTTCTTCAG | GTCTGGGTCTTTATGGNGCTTGTG | 136 | L29870 |
| 10 | D17S1655 | CGGACCAGAGTGTTCCATGG | GCATACAGCACCCTCTACCT | 240 | — |
| 11 | D17S2147 | AGGGGAGAATAAATAAAATCTGTGG | CAGGAGTGAGACACTCTCCATG | 138 | G15195 |
| 12 | D17S754 | TGGATTCACTGACTCAGCCTGC | GCGTGTCTGTCTCCATGTGTGC | 145 | — |
| 13 | D17S1814 | TCCCCAATGACGGTGATG | CTGGAGGTTGGCTTGTGGAT | 150-166 | Z52854 |
| 14 | D17S2007 | GGTCCCACGAATTTGCTG | CCACCCAGAAAAACAGGAGA | 102-103 | G07073 X03438 |
| 15 | D17S1246 | TCGATCTCCTGACCTTGTGA | TTGTCACCCCATTGCCTTTC | 115 | — |
| 16 | D17S1979 | CCTTGGATAGATTCAGCTCCC | CTTGTCCCTTCTCAATCCTCC | 199 | G11172 X55068 |
| 17 | D17S1984 | TTAAGCAAGGTTTTAATTAAGCTGC | GATTACAGTGCTCCCTCTCCC | 134 | G14779 T50487 |
| 18 | D17S1984 | GGTTTTAATTAAGCTGCATGGC | GATTACAGTGCTCCCTCTCCC | 126 | G11580 T50487 |
| 19 | D17S1867 | AGTTTGACACTGAGGGCTTTG | TTTAGACTTGGTAACTGCCG | 94 | Z51301 |
| 20 | D17S1788 | TGCAGATGCCTAAGAACTTTTCAG | GCCATGATCTCCCAAAGCC | 156-168 | Z52160 |
| 21 | D17S1836 | TCGAGGTTATGGTGAGCC | AAACTGTGTGTGTCAAAGGATACT | 167-173 | Z53182 |
| 22 | D17S1787 | GCTGATCTGAAGCCAATGA | TACATGAAGGCATGGTCTG | 239-251 | Z52130 |
| 23 | D17S1660 | CTAATATAATGCTGGGCACATGG | GCTGCGGACCAGACAGAT | 201 | G06069 |
| 24 | D17S2154 | GATAAAAACAAGCACTGGCTCC | CCCACGGCTTTCTTGATCTA | 137 | G15440 |
| 25 | D17S1955 | TGTAATGTAAGCCCCATGAGG | CACTCAACTCAACAGTCTAAAGGTG | 180 | G11900 |
| 26 | D17S2098 | GTGAGTTCAAGCATAGTAATTATCC | ATTCAGCCTCAGTTCACTGCTTC | 181 | G13994 |
| 27 | D17S518 | GATCCAGTGGAGACTCAGAG | TAGTCTCTGGGACACCCAGA | 88-100 | X60690 |
| 28 | D17S1851 | ATTCCTGAGTGTCTACCCTGTTGAG | ACTGACTGCGCCACTGC | 237-253 | Z53675 |
| 29 | D11S4358 | TCGAGAAGGACAAAATCACC | GAACAGGGTTAGTCCATTCG | 58 | — |
| 30 | D17S964 | GTTCTTTCCTCTTGTGGGG | AGTCAGCTGAGATTGTGCC | 224 | L36695 |
| 31 | D19S1091 | CAAGCCAAGACATCCCAGTT | CCCCACACACAGCTCATATG | 238 | G14589 |
| 32 | D17S1179 | TTTTCTCTCTCATTCCATTGGG | GCAACAGAGGGAGACTCCAA | 113-125 | — |
| 33 | D10S2160 | TCCCATCCCGTAAGACCTC | TATGGAGTACCTACTCTATGCCAGG | 349 | G06592 |
| 34 | D17S1230 | ATTCAAAGCTGGATCCCTTT | AGCTGTGACAAATGCCTGTA | 108 | L32949 |
| 35 | D17S1338 | TCACCTGAGATTGGGAGACC | AAGATGGGGCAGGAATGG | 178-200 | — |
| 36 | D17S2011 | TCACTGTCCTCCAAGCCAG | AAACACCACACTCTCCCCTG | 115 | G07143 |
| 37 | D17S1237 | TTCTTGGGCTTCCCGTAGCC | GGGGCAGACGACTTCTCCTT | 186 | L32947 |
| 38 | D17S2038 | GGGGATACAACCTTTAAAGTTCC | ATTCACCTAATGAGGATTCTTCTTT | 228 | G6219 |
| 39 | D17S2091 | GCTGAAATAGCCATCTTGAGCTAC | TCCGCATCCTTTTTAAGAGGCAC | 157 | G13941 |
| 40 | D17S649 | CTTTCACTCTTTCAGGTGAAGAGG | TGACGTGCTATTTCCTGTTTTGTCT | 146 | L36685 |
| 41 | D17S1190 | GTTTGTTGCTATGCCTGC | CAACACACTACCCCAGGA | 122 | L18197 |
| 42 | M87506 | ACTCCTCATCTGTAGGGTCT | GAGTCCGCTACCTGAGTGCT | 102-120 | m87506 |

1. A method for the prediction of response to cancer treatment, by the detection of at least 2 markers characterized in that the markers are genes and fragments thereof or genomic nucleic acid sequences that are located on one chromosomal region which is altered in malignant neoplasia.

2. The method of claim 1 wherein the treatment is an antibody treatment, antihormonal treatment, anti-growth factor treatment, taxol based treatment, anthracyclin based treatment and platinum salt based treatment.

3. The method of claim 1 wherein the treatment includes Herceptin™, trastuzumab or 2C4 antibodies.

4. The method of claim 1 characterized in that the markers are:
a) genes that are located on one or more chromosomal region(s) which is/are altered in malignant neoplasia; and
b)
i) receptor and ligand; or
ii) members of the same signal transduction pathway; or
iii) members of synergistic signal transduction pathways; or
iv) members of antagonistic signal transduction pathways; or
v) transcription factor and transcription factor binding site; or
vi) integral parts of heteromeric complexes

5. The method of claim 1 or 2 wherein the malignant neoplasia is breast cancer, ovarian cancer, gastric cancer, colon cancer, esophageal cancer, mesenchymal cancer, bladder cancer or non-small cell lung cancer.

6. The method of any of claims 1 to 5 wherein at least one chromosomal region is defined as the cytogenetic region: 1p13, 1q32, 3p21-p24, 5p13-p14, 8q23-q24, 11q13, 12q13,17q12-q24, 17q11.2-21.3 or 20q13.

7. A method for the prediction, diagnosis or prognosis of malignant neoplasia by the detection of at least one marker characterized in that the marker is selected from:
a) a polynucleotide or polynucleotide analog comprising at least one of the sequences of SEQ ID NO: 319 to 389;
b) a polynucleotide or polynucleotide analog which hybridizes under stringent conditions to a polynucleotide specified in (a) and encodes a polypeptide exhibiting the same biological function as specified for the respective sequence in Table 2 or 3
c) a polynucleotide or polynucleotide analog the sequence of which deviates from the polynucleotide specified in (a) and (c) due to the generation of the genetic code encoding a polypeptide exhibiting the same biological function as specified for the respective sequence in Table 2 or 3
d) a polynucleotide or polynucleotide analog which represents a specific fragment, derivative or allelic variation of a polynucleotide sequence specified in (a) to (d)
e) a purified polypeptide encoded by a polynucleotide or polynucleotide analog sequence specified in (a) to (e)
f) e purified polypeptide comprising at least one of the sequences of SEQ ID NO: 397-467;
Are detected.

8. The method according to any of claims 1 to 6 wherein the markers are selected from:
a) a polynucleotide or polynucleotide analog comprising at least one of the sequences of SEQ ID NO: 2 to 6, 8, 9, 11 to 16, 18, 19, 21 to 26, 53 to 76 or 315 to 389
b) a polynucleotide or polynucleotide analog which hybridizes under stringent conditions to a polynucleotide specified in (a) and encodes a polypeptide exhibiting the same biological function as specified for the respective sequence in Table 2 or 3
c) a polynucleotide or polynucleotide analog the sequence of which deviates from the polynucleotide specified in (a) and (b) due to the generation of the genetic code encoding a polypeptide exhibiting the same biological function as specified for the respective sequence in Table 2 or 3
d) a polynucleotide or polynucleotide analog which represents a specific fragment, derivative or allelic variation of a polynucleotide sequence specified in (a) to (c)
e) a purified polypeptide encoded by a polynucleotide sequence or polynucleotide analog specified in (a) to (d)
f) A purified polypeptide comprising at least one of the sequences of SEQ ID NO: 27 to 52 or 76 to 98 or 393 to 467
are detected.

9. A diagnostic kit for conducting the method of claims 1 to 8.
